(19) United States
(12) Patent Application Publication
(10) Pub. No.: US 2008/0166708 A1
(43) Pub. Date: Jul. 10, 2008

(54) Markers and Methods For Prenatal of Chromosal Alberrations

(76) Inventors: Nathalie Leporrier, Caen (FR); Michel Herrou, Gravus (FR); Sophie Feldblum, Paris (FR); Pierre-Yves Simonin, Le Havre (FR); Jean-Francois Cornuel, Paris (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/573,244

(22) PCT Filed: Aug. 8, 2005
(86) PCT No.: PCT/FR2005/002053

§ 371 Date: Aug. 21, 2007

(30) Foreign Application Priority Data
Aug. 6, 2004 (EP) 04292013.2

(51) Int. Cl. C12Q 1/68; C12Q 1/02; G01N 33/53

(52) U.S. Cl. 435/6; 435/29; 435/7.1

(57) ABSTRACT

The invention concerns prenatal screening of chromosomal aberrations, more particularly, through identification of novel markers of chromosomal aberrations, in particular, trisomy 21, whereof the assay based on a body fluid of the pregnant woman enables, alone or in combination with other assays, the probability that the fetus suffers from a chromosomal aberration to be more accurately determined than that by currently used tests.

The present invention relates to the prenatal screening of chromosomal aberrations. More particularly, the invention is based on the identification of novel markers for chromosomal aberrations, in particular trisomie 21, the assaying of which using a sample of biological fluid from the pregnant woman makes it possible, alone or in combination with other assays, to determine the probability that the fetus has a chromosomal aberration, with greater accuracy than that of the tests currently used.

Trisomie 21 or Down syndrome is the most common of the viable chromosomal aberrations. It causes psychomotor impairments and affects one birth in 800, and, given that approximately 800 000 births are observed in France, the incidence is 1000 children born with trisomie 21 each year.

Its characteristic phenotype allows clinical diagnosis at birth. Approximately 40% of children suffering from trisomie 21 also have malformations, the most common of which are congenital cardiopathies. Their life expectancy is now more than 50 years.

The risk of trisomie increases with maternal age, especially after 35 years of age (exponential growth). The risk is also increased if one of the parents is carrying an equilibrated translocation or if the couple has already given birth to a child with a chromosomal aberration. Fetal anomalies screened by echography can also lead to a fetal karyotype being prescribed.

Antenatal screening of trisomie 21 consists in studying the fetal karyotype, either on amniotic cells or on trophoblast cells, or on cord blood. The taking of the sample carries a risk of miscarriage. The karyotype is a long examination which is carried out in a specialized laboratory authorized by the Ministry of Health. It is therefore reserved for a population of women said to be “at high risk of chromosomal aberrations”.

The proportion of cases of trisomie 21 screened on the basis of criteria of maternal age and echographic warning signs is currently approximately one third. However, the prevalence of trisomie 21 at birth has not significantly decreased due to the regular increase in the average age of pregnant women.

Since 1980, there have been regulations (order of 29 Oct. 1991, published in the official journal of 16 Nov. 1991) governing whether fetal karyotyping is covered by health insurance. Fetal karyotyping is covered financially if one of the following indications is present:
1. the woman is 38 or more years old at the date on which the sample is taken,
2. parental chromosomal aberration,
3. family history, for the couple, of a pregnancy or pregnancies with aberrant karyotype,
4. diagnosis of the sex for sex-linked diseases,
5. the following echographic warning signs: internal or external morphological aberrations of the fetus demonstrated, established intrauterine growth retardation, amniotic fluid quantity anomalies.

For a few years, many teams have therefore been seeking to identify one or more serum markers that can be measured in maternal blood and that could allow a maternal serum-based evaluation of the risk on a wider population.

In 1984, a first maternal serum risk marker, alpha-fetoprotein (AFP), was identified: the AFP level, on average lower in pregnancies with trisomie 21, can be used to calculate the risk of trisomie. In 1987, another marker, chorionic gonadotrophin hormone (hCG) was found to be higher in pregnancies with trisomie 21. Since then, many other serum markers for a risk of trisomie 21 have been identified.

The markers most widely studied have been: hCG, AFP, unconjugated estriol (uE3), and hCG β-subunit. Each marker used individually is less effective than its combination with one or more others.

In France, a multicenter national pilot study and several regional studies were carried out. The demand from women gradually established itself and, in 1994, more than 10% of pregnant women were screened for serum markers.

Thus, in France, since 1997, maternal serum markers (hCG, uE3 and AFP) have been assayed and paid for by French Social Security. The test is listed as B145, i.e. 39 euros 15. To assay at least two of these markers, a blood sample is taken between the 15th and 18th week of amenorrhea, allowing a calculation of the risk to be made taking into account the risk a priori of the mother's age modulated according to the observed values for these markers. The threshold risk for making the decision to propose prenatal screening (amniocentesis) is arbitrarily fixed at 1/250.

The assaying of serum markers does not by itself establish the diagnosis of trisomie 21. It is a screening test that makes it possible to estimate the risk of the presence of a child with trisomie 21.

Evaluation of the risk of trisomie 21 by assaying serum markers in women who desire it is widely carried out today. In 1998, a study carried out by INSERM made it possible to estimate the number of assays at 572 000, i.e. 76% of births (definitely 85% in 2004).

This policy is also applied in the other European and Anglo Saxon countries where the frequency of trisomie 21 is the same.

The sensitivity of the screening is approximately 60% if the risk threshold chosen for access to amniocentesis is 1/250. This leads to approximately 5% of pregnant women being proposed an amniocentesis. The positive predictive value (specificity) is of the order of 1 to 2%. The screening sensitivity is better, at an equal amniocentesis rate, if the markers are more discriminating and combined with one another. The combinations most commonly used at the current time are the triple test (hCG+AFP+uE3) and the double tests, hCG+AFP or hCG+uE3, the latter being the best.

The false positives of the screening are aminocenteses with a normal karyotype. The number of false positives must be reduced as much as possible because of the risk of losing the fetus after amniocentesis (approximately 1%). Moreover, they can generate a great deal of anxiety in women awaiting the results of the karyotype.

The false negatives of the screening are cases of trisomie 21 not revealed by the screening. They worsen the emotional shock at the time of birth. This is because couples who have had the benefit of a serum marker assay understand poorly the notion of residual risk and often think that the test is a diagnostic means. The failure of the screening is therefore a severe blow.

This situation is not satisfactory since it allows 30 to 40% of pregnancies with trisomie 21 to get through. It is necessary to find a technique aimed at increasing the sensitivity and specificity, reducing the number of needlessly invasive procedures (false positives) and especially reducing the number of false negatives (30 to 40%).

Other procedures are in the process of being evaluated and are not reimbursed by French Social Security, in particular serum markers for the first trimester of pregnancy. These are the PAPP-A protein (pregnancy-associated plasma protein A), the free beta-subunit of hCG and the measurement of nuchal translucency at 12 weeks of amenorrhea. Some teams are also trying to develop the demonstration of fetal cells or of free fetal DNA in maternal blood, but have not, at this time, succeeded in producing an effective screening.

The effectiveness of the serum markers depends on the strategy chosen: risk threshold above which an amniocentesis is proposed, nature of the markers used, and type of inter-combinations. Certain combinations of markers are more effective than others (Wald et al., Lancet, 2003, 8, 361, 855-6).

A recent study carried out on 120 000 pregnancies has shown that fetuses with trisomie 21 detected prenatally by means of serum markers or echo-graphic aberrations (nuchal translucency) would have spontaneously aborted more than the fetuses with trisomie not detectable by these methods (Leporrier N. et al., BJOG, 2003, 110, I, 18-21). This therefore decreases the effectiveness of this screening and prompts a search for other more effective biomarkers.

In an original approach aimed at discovering new biomarkers that are more specific for trisomie 21, and/or at determining a specific expression profile, based on several molecules, the inventors have carried out studies based on 280 frozen sera from pregnant women with fetuses with trisomie 21 (including those that were detected and those that were not detected by the second-trimester serum markers), and more than 280 control sera from women that did not have fetuses with trisomie 21, the outcomes of which are known.

The placenta is a barrier between the mother and the fetus, but it has been proved that certain molecules of low molecular mass cross this barrier. The inventors have therefore chosen to apply the proteomic technique to the serum of pregnant women, in order to determine a differential expression profile that makes it possible to distinguish, in a virtually definite manner, those with fetuses with trisomie 21 in comparison with sera from normal pregnant women. This approach, illustrated in the experimental section hereinafter, has allowed them to identify several markers that make it possible to distinguish, in a virtually definite manner, the sera of women with fetuses with trisomie 21 in comparison with sera from pregnant women carrying normal fetuses.

The present invention therefore relates, firstly, to the use of one or more marker(s) chosen from (i) the human neutrophil defensins HNP-2, HNP-1 and HNP-3, and/or (ii) the molecular species detectable by mass spectrometry with separation of anion exchange type, at the following molecular weights: 4461.60 Da; 4570.90 Da; 4630.70 Da; 4714.30 Da; 4859.10 Da; 8195.65 Da; 8905.85 Da; 9121.00 Da; 9700.50 Da, and/or (iii) the molecular species detectable by mass spectrometry with separation of hydrophobic type, at the following molecular weights: 2625.47 Da; 4068.5 Da; 4075.95 Da; 4084.3 Da; 4084.7 Da; 4093.13 Da; 4320.79 Da; 4642.66 Da; 4658.47 Da; 6419.80 Da; 8107 Da; 8124.5 Da; 8140.5 Da; 8155 Da; 8269.10 Da; 8338.80 Da; 8610.30 Da, for the prenatal diagnosis of a fetal aberration.

The term “molecular species” here denotes, inter alia, any glycosylated or nonglycosylated, phosphorylated or nonphosphorylated protein, any protein complex, any product of metabolism, or any fragment of any one of these entities.

The molecular weights indicated here were obtained under experimental conditions specified below. Certain technologies, different than those used by the inventors, that can be used to implement the invention are mentioned hereinafter. It is clear that a certain amount of variation is liable to appear during the use of different technologies and/or equipment. Nevertheless, it is also very clear that those skilled in the art are capable of transposing the results described in the experimental section below to other technologies, with a satisfactory degree of cover, and of identifying without difficulty the signals (peaks, etc.) corresponding to the markers demonstrated by the inventors. Due to this technology-related variation (also related to the specifications of manufacturers of spectrometry devices, which in particular use information processing programs that may lead to peak shifts), the molecular weights indicated above should be understood with an accuracy of ±20%, preferably ±10%, or even ±5%, and, under good conditions, ±1%. It should be noted that, under the conditions described in the experimental section hereinafter, the measurement of the molecular weights of the various markers is reproducible, with a variation coefficient much lower than 10%, typically of the order of 0.3% to 0.5%, for the markers of molecular weight less than 12 000 Da. An accuracy of ±0.1% is even obtained up to 6000 Da. Due to the calibrating agents used, this variation coefficient is of the order of 10 to 25% for the markers of molecular weights greater than 12 000 Da. As specified in the experimental section below (title C-3), the choice of calibrating agent does not influence the observed peak intensity values. The use of calibrating agents other than those used by the inventors may possibly lead to a variation in terms of the mass measured for the marker, the peaks corresponding to each of the markers still remaining, however, identifiable.

The invention also relates to an in vitro screening method for determining whether a pregnant woman is carrying a fetus suffering from an aberration, comprising a step consisting in detecting or in assaying, in a biological sample from said pregnant woman, at least one marker selected from any one of groups (i) to (iii) defined above.

For the purpose of the present invention, the word “assay” denotes a quantitative or semiquantitative measurement in the broad sense, for example a relative quantification of a compound, compared to the same compound in another sample, or compared to another compound in the same sample. Thus, in the present text, a comparison of profiles, obtained for example by mass spectrometry, is likened to an “assay”.

In a preferred implementation of the method of the invention, it comprises a step consisting in assaying at least two or three markers, at least one of which is selected from groups (i) and/or (ii) and/or (iii) above, and the other or the two others of which is (are) selected either from these same groups or from the markers already characterized and used today, here constituting group (iv), comprising the second-trimester markers—chorionic gonadotrophin hormone (hCG) or its free β-subunit, unconjugated estriol (uE3), α-fetoprotein (AFP) and/or inhibin A—or the first-trimester markers—free β-subunit of hCG and/or pregnancy-associated plasma protein (PAPP-A). Alternatively, one of the assays can be replaced with the observation of nuchal translucency, in the first trimester.

For example, an “enhanced triple test”, based on assaying hCG, uE3 and one of the markers mentioned above, is an integral part of the invention. Alternatively, the assaying of a marker of groups (i) to (iii) above can be combined with one or more other known test(s), for instance the nuchal translucency test.

The method of the invention involves a step consisting in assaying from a biological sample originating from the pregnant woman, which sample may consist of any body fluid, such as a blood sample, serum sample, plasma sample, urine sample, etc.

According to various variants of the invention, the number of markers selected from the molecules identified by the inventors may be equal to 1, 2, 3, 4, 5, 6, 7, 8, 9, 10, 15, 20 or more, irrespective of whether they are markers selected from group (i), (ii), (iii) or (iv) (at least one of the markers being selected from one of groups (i) to (iii)). Those skilled in the art will determine the number of markers to be assayed according to effectiveness constraints (search for a test that is as sensitive and selective as possible), and economic constraints.

In a preferred embodiment of the methods of the invention, these methods also comprise, for each marker assayed, a step consisting in comparing the concentration of this marker measured in the biological sample from the pregnant woman, with reference values for the concentration of this marker in pregnant women carrying normal fetuses, and in pregnant women carrying fetuses suffering from a known chromosomal and/or genetic aberration, the comparison being indicative of the risk that the pregnant woman is carrying a fetus suffering from a chromosomal and/or genetic aberration. These references will be chosen by those skilled in the art so as to correspond to the gestational age of the sample taken from the pregnant woman.

In a particular embodiment of the methods of the invention, illustrated in the examples, the assaying of at least one marker is carried out by mass spectrometry, for example by low-resolution or high-resolution mass spectrometry.

Of course, other separation, purification or assaying techniques can be used in the context of the invention. In particular, two types of technologies can be envisioned for developing a diagnostic device according to the invention: profile comparison, or isolated assaying of markers. By way of nonlimiting examples, mention may be made of the following techniques:

Separation techniques: HPLC, LC (Liquid Chromatography), CE (Capillary Electrophoresis), which are all technologies of “microfluidic” type, two- or one-dimensional gels, reverse-phase separation on a planar or nonplanar solid support (planar surface, bead surface), it being possible for biological samples to be prefractionated, for example on a membrane (Vivaspin type from Vivascience, but also spin columns from Millipore, Proteospin type), and/or depleted of the major proteins (albumin, ovalbumin, immunoglobulins, etc.), and/or affinity-associated, beforehand.

Assaying by profile comparison (mass spectra): in addition to the technologies described in the experimental section, mention may be made, by way of example of a mass spectrometer that can be used in the context of the invention, of the ClinProt™ system developed by the company Bruker Daltonics, which uses magnetic microparticles with a functionalized surface.

In the case of the profile-comparison assays, several methods of detection of the signal can be used, alone or coupled with separative methods (Technologies TOF, laser-MS and MS-MS, ESI-MS, LC-MS, MALDI, tandem, Q-Trap, etc., SELDI-MS, SELDI-MS-MS, chemiluminescence (Mesoscale, Roche diagnostics), Elecsys technology, fluorescence, ICAT, piezoelectric signal, optical signal such as surface plasmon resonance or SPR, etc.), to reveal and assay the molecules derived from the profiles, either in native form, or in ionic form (1+, 2+, 3+, 4+, 5+, etc.), applicable to proteins, to glycoproteins and to lipoproteins.

According to a specific embodiment of the method of the invention, said method comprises a step consisting in analyzing at least one spectrum obtained from a biological sample originating from a pregnant woman; according to this variant, the signal of at least 2 peaks chosen from (i) the peaks corresponding to the human neutrophil defensins HNP-2, HNP-1 and HNP-3, and/or (ii) the peaks that can be observed by mass spectrometry with separation of anionic type, at the following molecular weights: 4461.60 Da; 4570.90 Da; 4630.70 Da; 4714.30 Da; 4859.10 Da; 8195.65 Da; 8905.85 Da; 9121.00 Da; 9700.50 Da, and/or (iii) the peaks that can be observed by mass spectrometry with separation of hydrophobic type, at the following molecular weights: 2625.47 Da; 4068.5 Da; 4075.95 Da; 4084.3 Da; 4084.7 Da; 4093.13 Da; 4320.79 Da; 4642.66 Da; 4658.47 Da; 6419.80 Da; 8107 Da; 8124.5 Da; 8140.5 Da; 8155 Da; 8269.10 Da; 8338.80 Da; and 8610.30 Da, is measured.

As explained above, the term “spectrum” here denotes any type of spectrum (mass, MALDI, MALDI-TOF, SELDI, SELDI-TOF, surface plasmon resonance, liquid chromatography, ICAT, ESI, ESI-TOF, etc., spectrum), those skilled in the art being capable of transposing the mass spectrometry results presented hereinafter to another technology and of finding the relevant peaks. Similarly, the term “signal” denotes any observable factor (statistical variable) or ratio that can be observed, such as the intensity, the signal-to-noise, the Tof (time of flight) area, the Tof width, the resolution, etc.

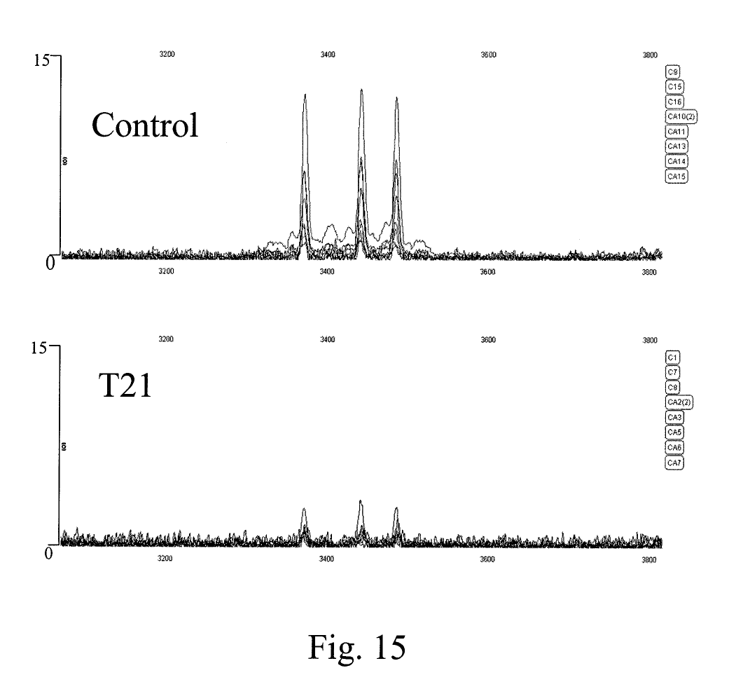

Among the markers characterized by the inventors, three have been identified as being the human neutrophil defensins HNP-2, HNP-1 and HNP-3 (see experimental section, D-9). These defensins are over-expressed or normally expressed in the controls, and underexpressed in the case of trisomie 21 (FIG. 15). According to a preferred embodiment of the method of the invention, at least one of the markers for which the assay is carried out is selected from HNP-2, HNP-1 and HNP-3. A high level of the marker in question indicates a normal fetus, whereas a low level indicates a high probability of trisomie 21.

In addition, and particularly surprisingly and advantageously, the inventors have noted that the ratios between these defensins HNP-2, HNP-1 and HNP-3 vary depending on whether the fetus is or is not suffering from Down syndrome. As explained in detail in the experimental section below (section F), the longitudinal ratios HNP-1/HNP-3 and HNP-2/HNP-3 are statistically higher in the case of trisomie 21 than when the pregnant woman is carrying a nontrisomic fetus. These ratios, which are particularly indicative, also have the advantage of being independent of the calibration of the measuring device. The inventors have, moreover, noted that the level of expression of the HNP2, HNP1 and HNP3 defensins, and also the ratios between these various levels, correlate neither with the age of the pregnant woman, nor with the week of pregnancy, nor with any of the markers currently used (AFP, uE3 and HCG, in particular), nor with any of the other markers identified in the studies reported here.

Thus, according to a preferred embodiment of the method of the invention, the ratio between the concentrations of HNP-3 and of HNP-1 and/or HNP-2 in the sample is measured.

Of course, these ratios can be measured from a spectrum obtained according to any one between the techniques mentioned above. It is also possible to measure the defensin expression ratios by means of a nucleic acid amplification technique, for example by quantitative or semiquantitative RT-PCR, using the biological sample or using RNA extracted from this sample. Primers that can be used for this are, for example, described in the article by R. M. Linzmeier, and T. Ganz (“Human defensin gene copy number polymorphisms: Comprehensive analysis of independent variation in alpha- and beta-defensin regions at 8 p 22-p 23” Genomics 2005).

Alternatively, it is also possible to measure, in the biological sample, the total sum of the HNP-1, HNP-2 and HNP-3 defensins, which are all underexpressed in the case of trisomie 21. According to a specific embodiment of the method of the invention, the first marker detected is therefore the sum of the HNP-2, HNP-1 and HNP-3 defensins. This detection can in particular be carried out by means of immunological techniques, for example by ELISA or PCR.

In general, the use of one or more human neutrophil defensins chosen from HNP-1, HNP-2 and HNP-3 (also referred to as HND-1, HND-2 and HND-3), as a marker or markers for the prenatal diagnosis of a chromosomal aberration, is therefore an integral part of the present invention.

As mentioned above, it is possible to implement the methods of the invention by carrying out an isolated assay of at least some markers. By way of nonlimiting examples of technologies that can be used for this (which can also be coupled to the chromatographic technologies mentioned above), mention may be made of the following technologies:

Antibodies can be produced against the markers of the invention, by any technique, including monoclonal antibodies, chemical synthesis, biological production (via animals [example Bioprotein, etc.], via plants [example Lemna—Biolex-Bayer, Meristems Therapeutics also, etc.]). They can then be used either to directly carry out an immunoassay of the corresponding markers, or to purify the markers prior to them being assayed.

Immuno assay (or IA): various technologies known to those skilled in the art can be envisioned, such as the sandwich immunoassay (sandwich IA), surface interaction (surface plasmon resonance, or SPR) or immunoprecipitation (for example, the Q-MAP technology developed by Atto-Lab).

The sandwich IA approach is based on the double recognition of the marker—or antigen—by specific antibodies: “capture” antibodies immobilized on the chosen support (microtitration plate, magnetic bead, etc.), and “detection” antibodies, in solution and coupled to a reactive molecule, responsible for the emission of the binding signal (directly or indirectly). This set of nonoverlapping specific antibodies (Pair Matched Antibodies) constitutes the reagents common to the current various detection systems.

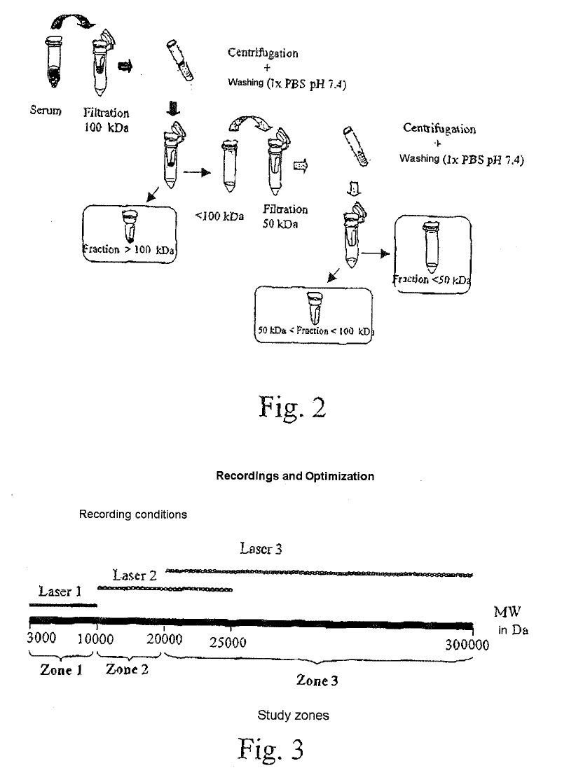

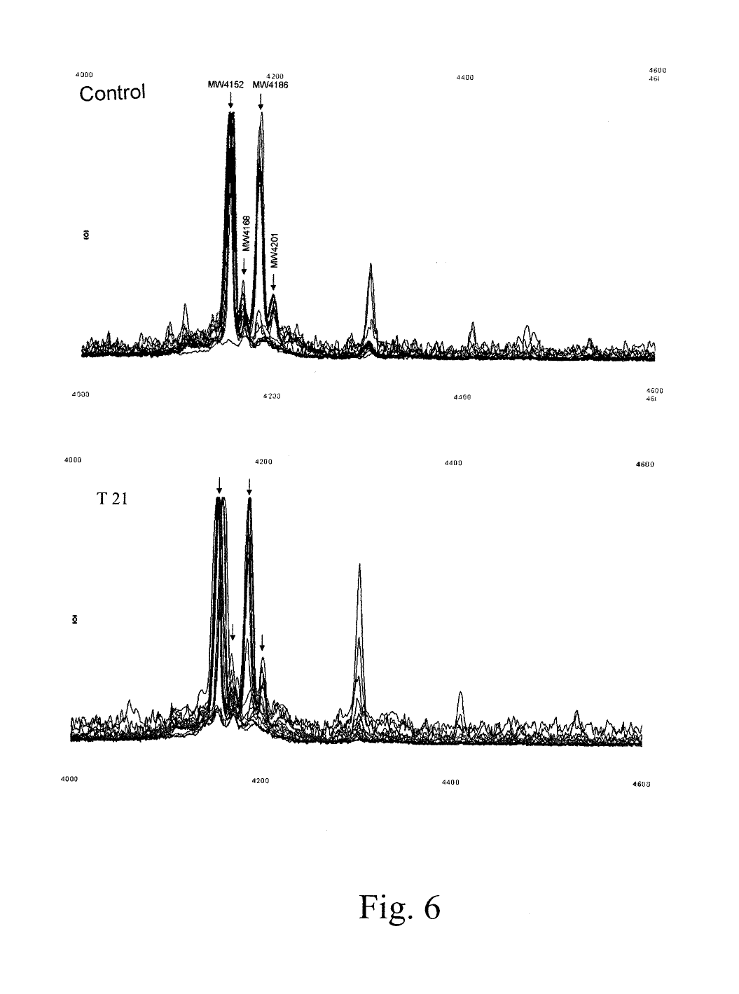

Alternatively, the antigen recognition can be carried out using aptamers, which are single-stranded oligonucleotide sequences (DNA or RNA) selected in vitro for their ability to bind to a given molecule; this selection can be carried out, for example, by the SELEX method (Systematic Evolution of Ligands by Exponential Enrichment) as described in U.S. Pat. No. 5,270,163. A functionalized aptamer can, for example, be used in place of a secondary antibody in an immunoassay of “Sandwich ELISA” type, as described by Sumedha Jayasena (Clinical Chemistry, 1999, 45(9): 1628-50, FIG. 6). The aptamers can be subjected to optimization by various modifications (Sumedha Jayasena, above, FIG. 2). It is in particular possible to modify the aptamers such that UV-activation after binding of their target ensures covalent bonding of this target to the support (photoaptamers).

The various components of an immunoassay-type diagnostic device also depend on the detection system used. Some reagents and supports that can be used to implement the invention are summarized in table 1 below, along with suitable examples of instrumentation.

TABLE 1
the various components of an IA-type diagnostic device

| | Reagents (except | | |
|---|---|---|---|
| | capture and | | |
| | detection | | Instrumentation |
| | antibodies or | | (photomultipliers |
| | aptamers and | | and CCD cameras |
| | associated | | for IFA, CLIA |
| Detection system | buffers) | Support | and ECLIA) |
| RIA | Radioisotopes(¹²⁵I) | Microplates, | Technicon Star |
| (Radioimmuno- | | microbeads, | System, etc. |
| assay) | | magnetic | |
| IFA (immuno- | Fluorophores | microbeads | DELFIA (Perkin), |
| fluorescent | (Aqualite, | | etc. |
| assay), including | lanthans, FITC, | | |
| the FP | PE, etc.) | | |
| (fluorescence | | | |
| polarization), | | | |
| TRF (time | | | |
| resolved | | | |
| fluorescence) and | | | |
| FRET | | | |
| (fluorescence | | | |
| resonance energy | | | |
| transfer) | | | |
| techniques | | | |
| CLIA (chemi- | Enzymes (AP, HRP, | | Advia Centaur |
| luminescence | etc.) | | (Brahms), AxSym |
| assay) | Substrate | | (Abbott), |
| | (acridium, | | Immulite 2000 |
| | cryptate, etc.) | | (DPC), etc. |
| ECLIA | Substrate | | Elecsys 2010 |
| (electrochemi- | (ruthenium) | | (Roche), etc. |
| luminescence | | | |
| assay | | | |

Protein chips can be designed for determining chosen protein expression profiles involving the desired markers. This technology is presented in the review article by Sydor and Nock (Proteome Science, 2003, Jun. 10; 1(1): 3). Of course, those skilled in the art can improve the technologies presented in this article by using their general knowledge in this field.

By way of specific examples of innovative immunoassay technologies that can be used in the context of the invention, mention will be made of MAP technology (Luminex Corp), Bio Bar Codes (Nanotechnologies Inc.), the markers LumiPhos 530 and 480 (Lumigen) and RCA (Rolling-Circle Amplification).

MAP technology (Luminex Corp) allies flow cytometry and microparticles in a format of IFA (Immunofluorescent Assay) type. This technology proposes an analysis by double fluorescence. The first detection system makes it possible to capture the fluorescence emitted by the beads that capture the markers (one marker and one fluorescence correspond to one bead); the second makes it possible, for its part, to measure the fluorescence associated with the detection antibodies, i.e. the quantification of the markers.

Bio Bar Codes (Nanotechnologies Inc.) ally nanoparticles and scanometry (Argent), introducing a new detection system based on the hybridization of unique nucleic acid probes (identification of the markers) (Nanoparticle-Based Bio-Bar Codes for the Ultrasensitive Detection of Proteins, Jwa Min Nam et al., Science 2003).

The markers LumiPhos 530 and 480 (Lumigen) combine chemiluminescent and fluorescent detection systems, thereby ensuring optimization of the signal.

RCA (Rolling-Circle Amplification) allows the detection of proteins with a sensitivity of the order of a zeptomole (10⁻²¹ moles). This technology, described by Schweitzer et al. (Proc. Natl. Acad. Sci. USA, 2000 Aug. 29; 97(18): 10113-9), involves the polymerization of a long single-stranded DNA chain attached to the analyte (for example, an antibody), using a circular DNA template. This technology makes it possible to minimize sample volumes and the use of expensive materials (monoclonal antibodies and enzymes).

The possibility of using signal amplification systems as another category of reagents in the context, in particular, of IFA and CLIA (for example: biotin/avidin) will also be noted.

The markers can also be detected by means of a chain polymerization nucleic acid amplification technique (PCR or other technique).

The invention makes it possible to screen chromosomal aberrations, in particular Down syndrome (trisomie 21), Edwards syndrome (trisomie 18), Patau syndrome (trisomie 13), Turner syndrome, Klinefelter syndrome, monosomie X, fragile X syndrome, penta X syndrome, and a deletion of the long arm of chromosome 7, but also other congenital aberrations, currently screened by echography, for example Spina Bifida. In addition, the invention can be applied to the screening of genetic, infectious (viral, bacterial or parasitic) or metabolic pathologies of the fetus, or to the search for a specific phenotype, and in particular the sex of the fetus. The invention is particularly suitable for the prenatal diagnosis of Down syndrome.

The present invention is applicable to prenatal screening during the second trimester, or earlier, during the first trimester of pregnancy.

Of course, the general knowledge of those skilled in the art will allow them to supplement the methods of the invention, for example by taking into account the age of the pregnant woman or any other variable (week of gestation, etc.) used as an adjustment criterion.

The screening methods according to the invention can also comprise an initial step consisting in preparing the sample. For example, this step can comprise a fractionation according to the mass of the proteins, and/or a step consisting of depletion of albumin or other transport proteins, and/or a step consisting in purifying the proteins on a preparative surface of anionic and/or hydrophobic type.

The present invention also relates to a kit for the prenatal diagnosis of chromosomal aberrations, comprising suitable means for carrying out one of the methods described above.

Those skilled in the art are capable of selecting, according to the assaying and detection techniques that they wish to use for implementing the invention, the elements to be integrated into such a diagnostic kit: specific antibodies, secondary antibodies, aptamers, detection reagents (fluorescent compounds, enzymes, antibodies modified for using RCA technology, etc.), primers and/or probes, etc. They can choose at least some of these elements from the elements mentioned above, and can complete the kit, where appropriate, using their general knowledge and reference works regarding the purification, assaying or detection of biological molecules.

A prenatal diagnostic kit according to the invention can, for example, comprise specific antibodies and/or aptamers for one or more markers mentioned above and/or amplification primers and also probes. Of course, in a kit that contains several antibodies, aptamers or primers specific for markers, each antibody, aptamer or primer is specific for a single marker. When these compounds are in solution, each solution is preferably specific for a single marker, or for a group of associated markers (for example, the HND-1, HND-2 and HND-3 defensins). By way of examples of antibodies that can be included in a kit according to the invention, mention may be made of anti-HND antibodies such as those sold by Tebu-Bio SA (39 rue de Houdan, F-78612 Le Perray en Yvelines) under the reference 038NCL-DEFENSIN (Anti Neutrophil Defensins Mouse IgG1 Clone D21, 1 ml).

Advantageously, a diagnostic kit according to the invention comprises, for at least one of the markers of the invention, a set of nonoverlapping specific antibodies, i.e. at least two antibodies capable of simultaneously recognizing the marker in question, which makes it possible to carry out sandwich-type immunoassays.

The antibodies, in particular the “capture” antibodies, can be immobilized on a support, and the “detection” antibodies can be coupled to a molecule for detection. Nonlimiting examples of supports and of molecules for detection, that can be used in the context of the invention, are mentioned above.

In addition to the above arrangements, the invention also comprises other arrangements that will emerge from the description which follows, which refers to examples of implementation of the method which is the subject of the present invention and also to the attached drawings, in which:

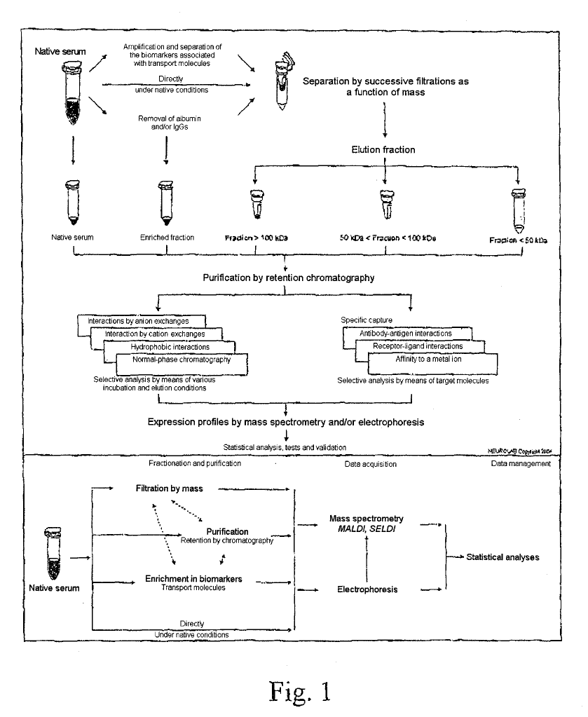

FIG. 1 illustrates the experimental strategy used;

FIG. 2 illustrates a schematic preparation of the samples;

FIG. 3 summarizes the various recording conditions, according to the molecular mass of the proteins to be detected;

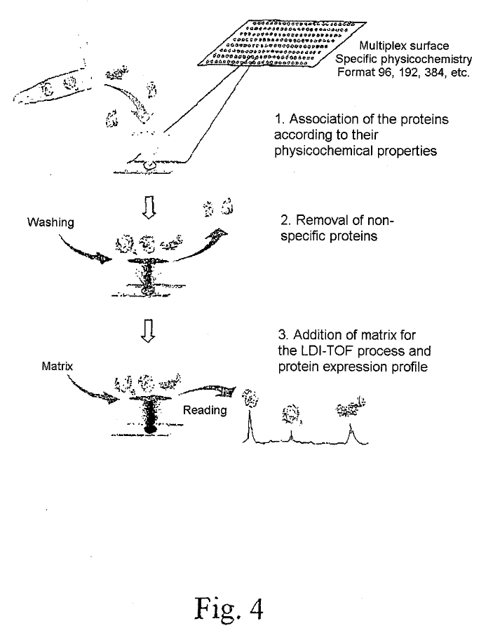

FIG. 4 illustrates the experimental scheme used in the studies E1, E2, E3, E4 and E6;

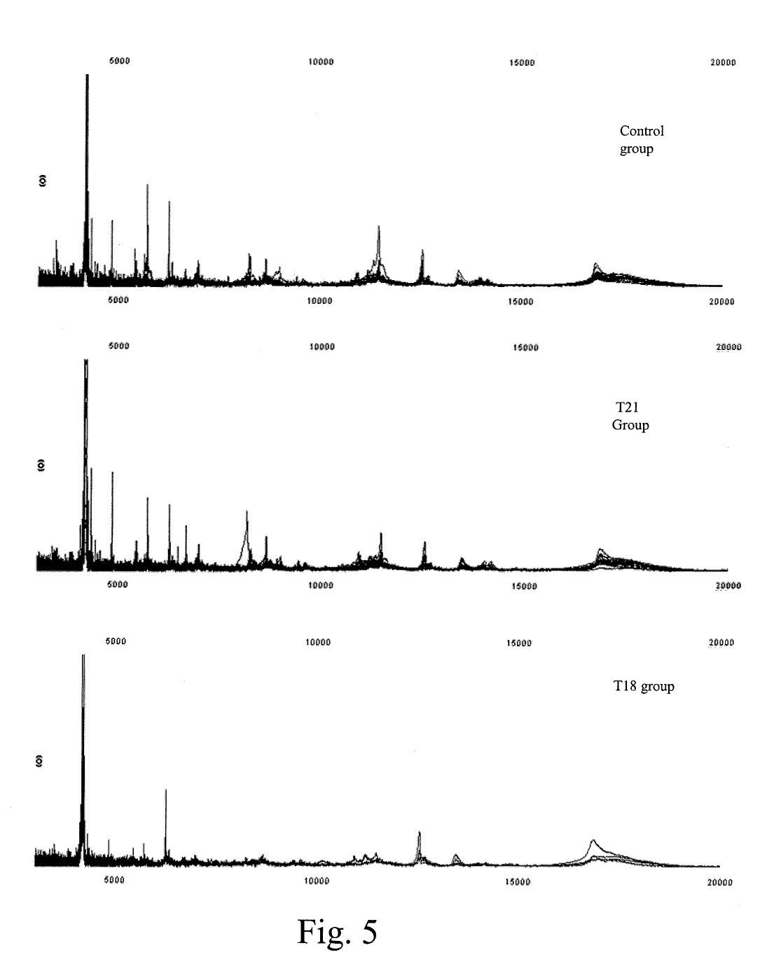

FIG. 5 represents the spectra obtained over the 3000-20 000 dalton spectral zone, in overall views, for the control group, the T21 group and the T18 group;

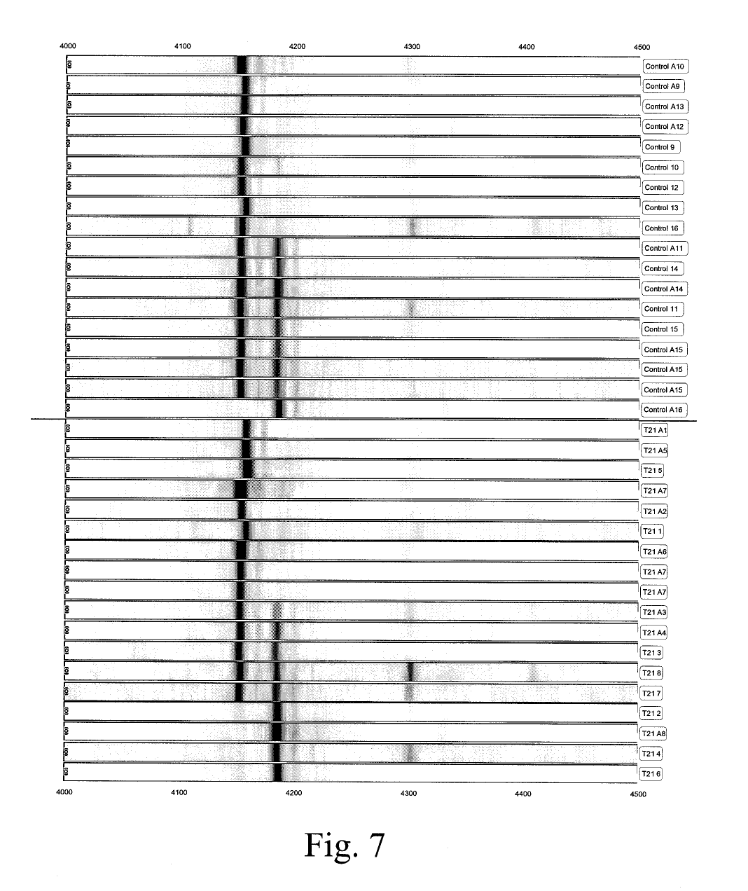

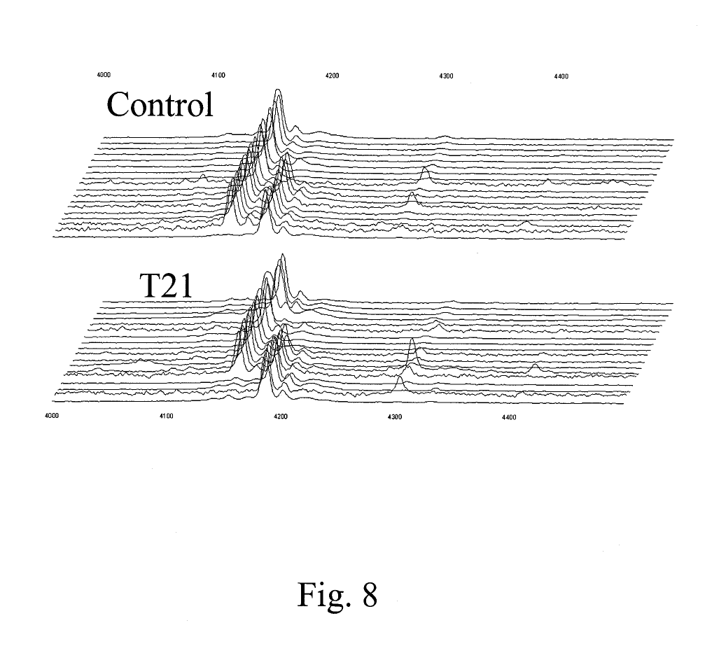

FIGS. 6-8 illustrate the spectra centered on the 4000 dalton zone (FIG. 6: spectra as overall views; FIG. 7: pseudogels; FIG. 8: spectra as shifted views);

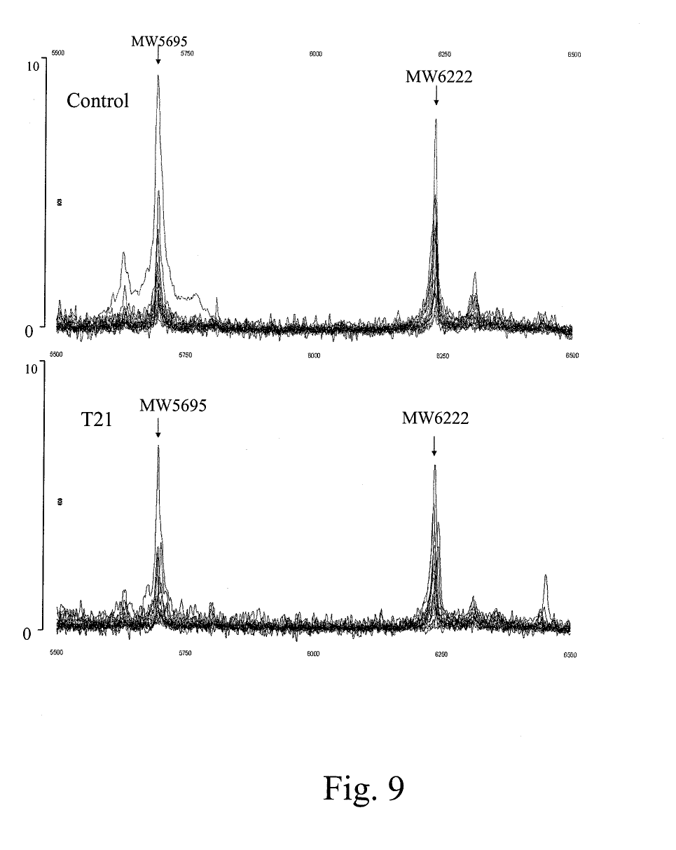

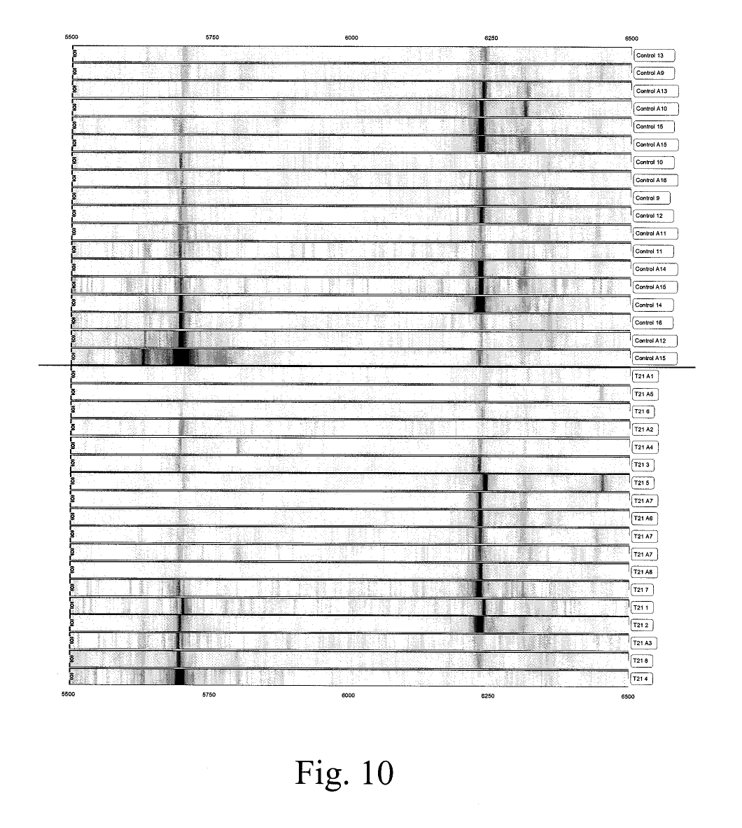

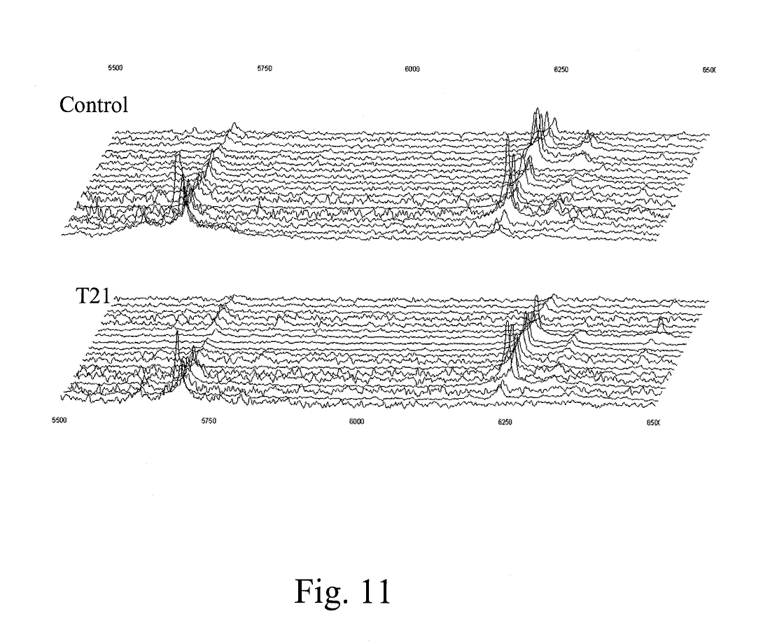

FIGS. 9-11 illustrate the spectra centered on the 5000-6000 dalton zone (FIG. 9: spectra as overall views; FIG. 10: pseudogels; FIG. 11: shifted views);

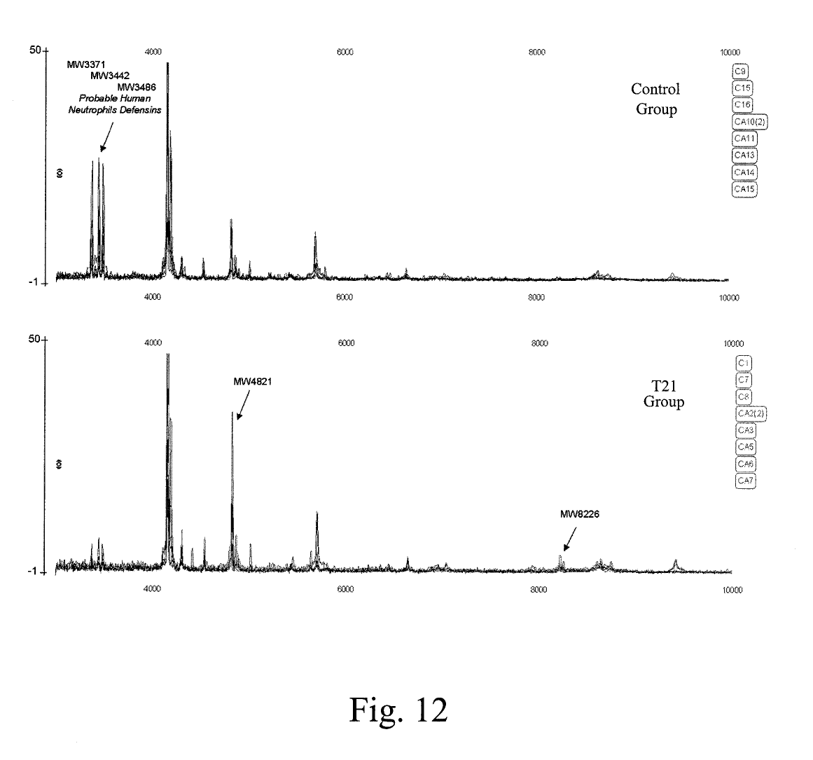

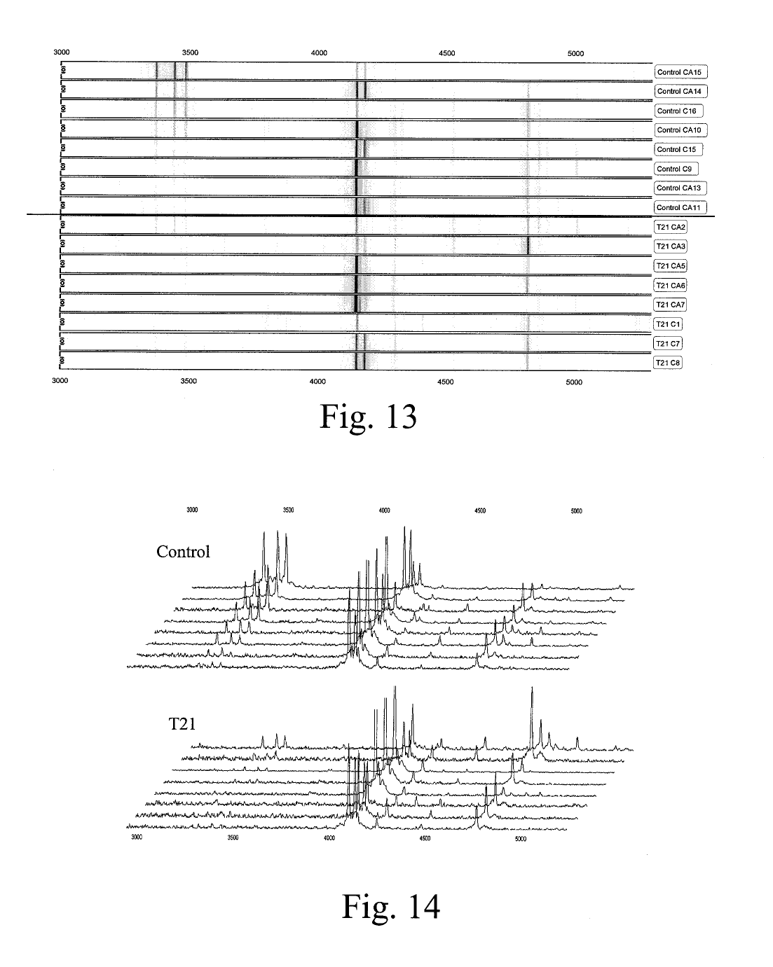

FIGS. 12 to 14 illustrate the spectra centered on the 3000-10 000 dalton spectral zone (FIG. 12: spectra as overall views; FIG. 13: pseudogels; FIG. 14: shifted views);

FIG. 15 illustrates the spectra centered on the 3000-4000 dalton zone (spectra as overall views);

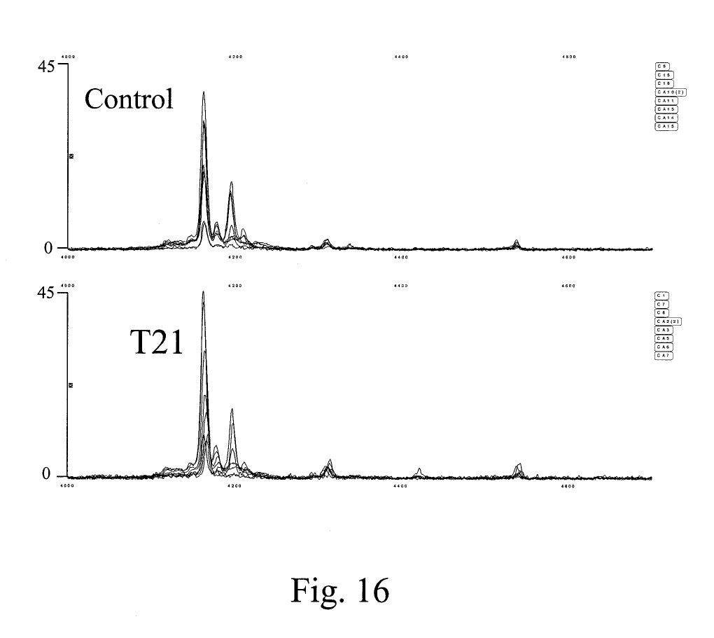

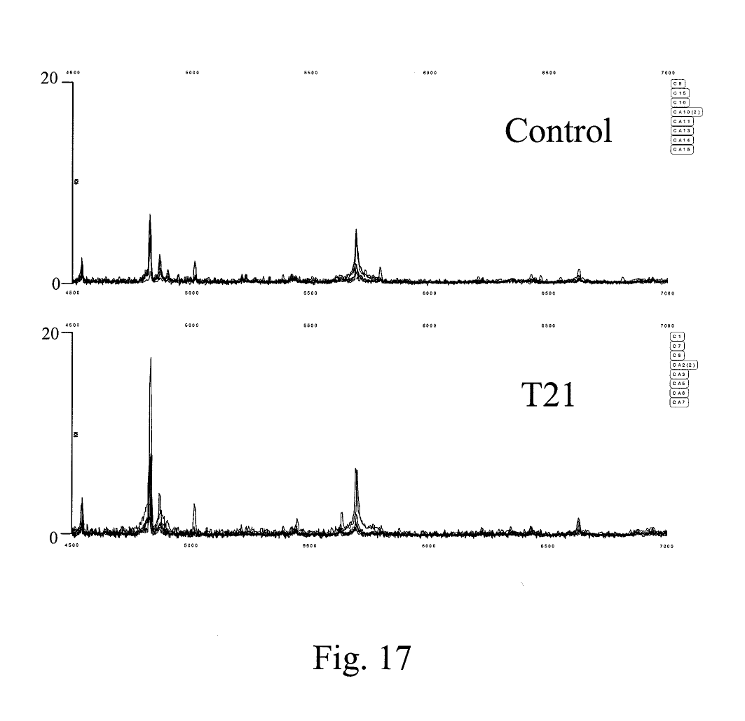

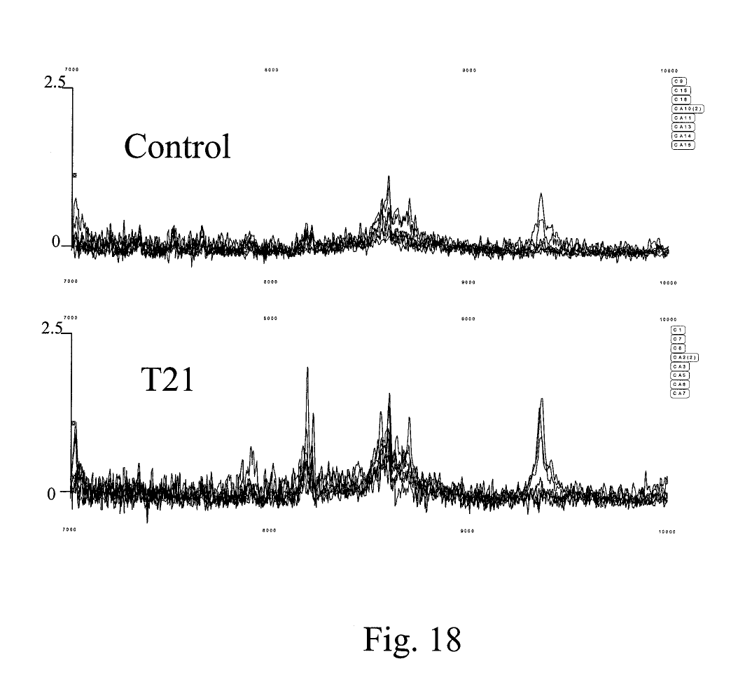

FIGS. 16-18 illustrate the spectra centered, respectively, on the 4000-5000 dalton, 5000-7000 dalton and 7000-10 000 dalton zones (spectra as overall views);

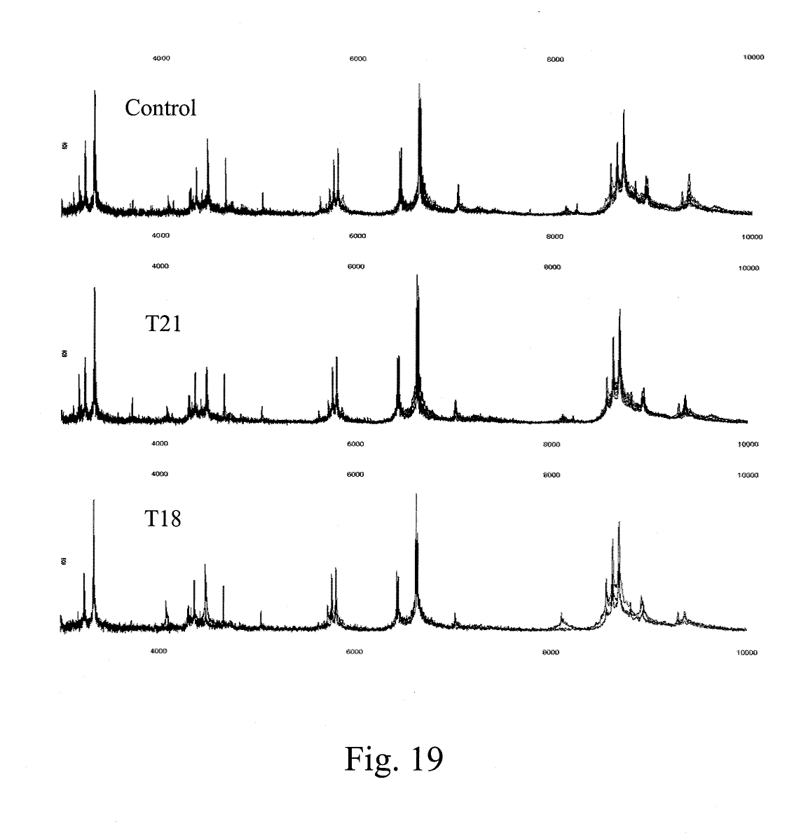

FIG. 19 illustrates the spectra as overall views, normalized and superimposed per clinical category, for the control group, the T21 group and the T18 group, over the 3000-10 000 dalton spectral zone (study E6);

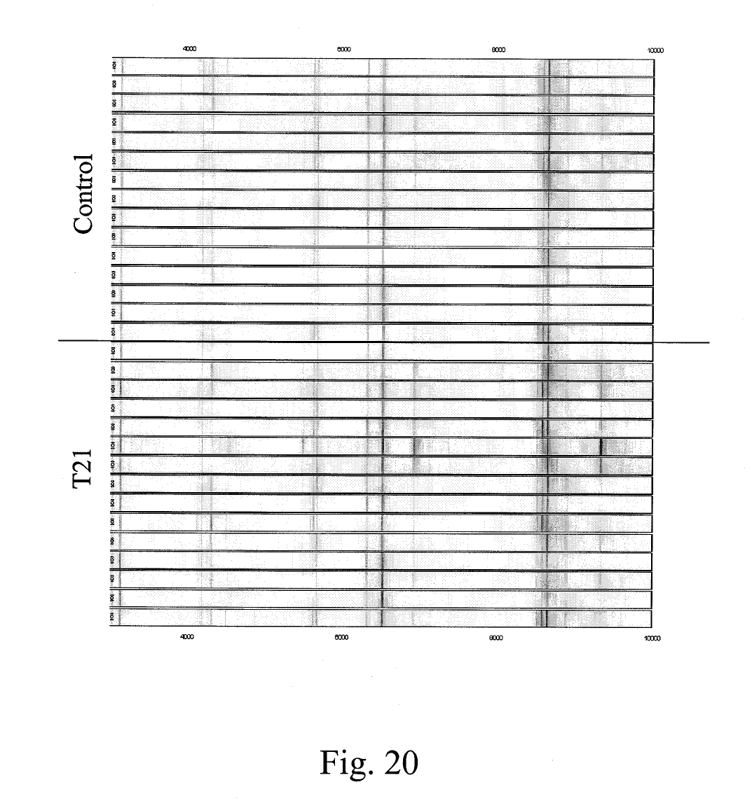

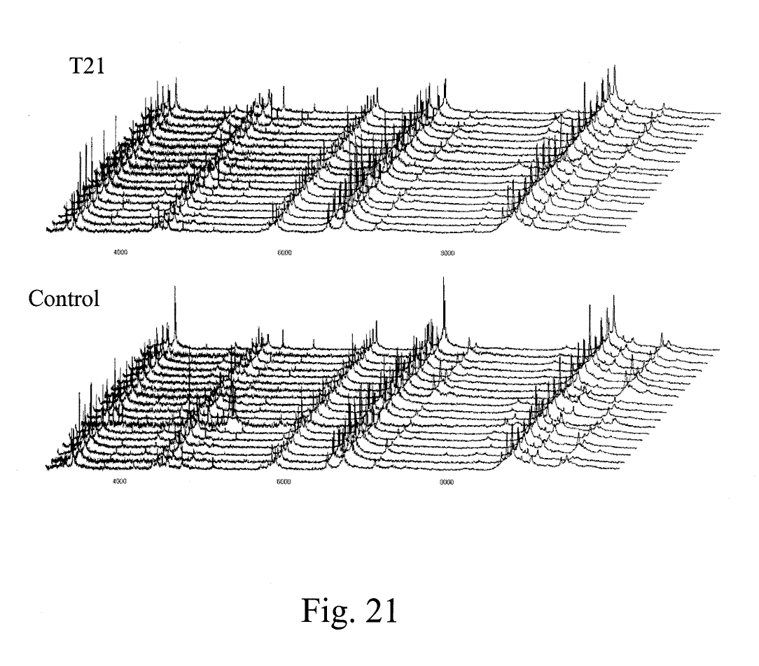

FIGS. 20 and 21 illustrate, for the same spectral zone as that of FIG. 19, the pseudogel views (FIG. 20) and the shifted views (FIG. 21) only for the control group and the T21 group;

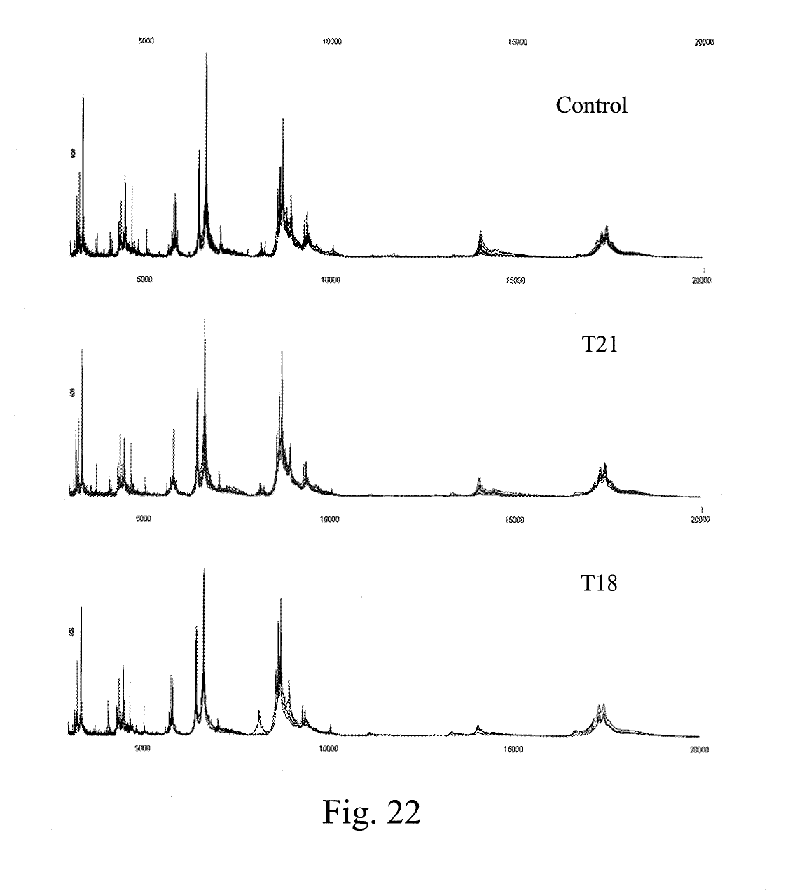

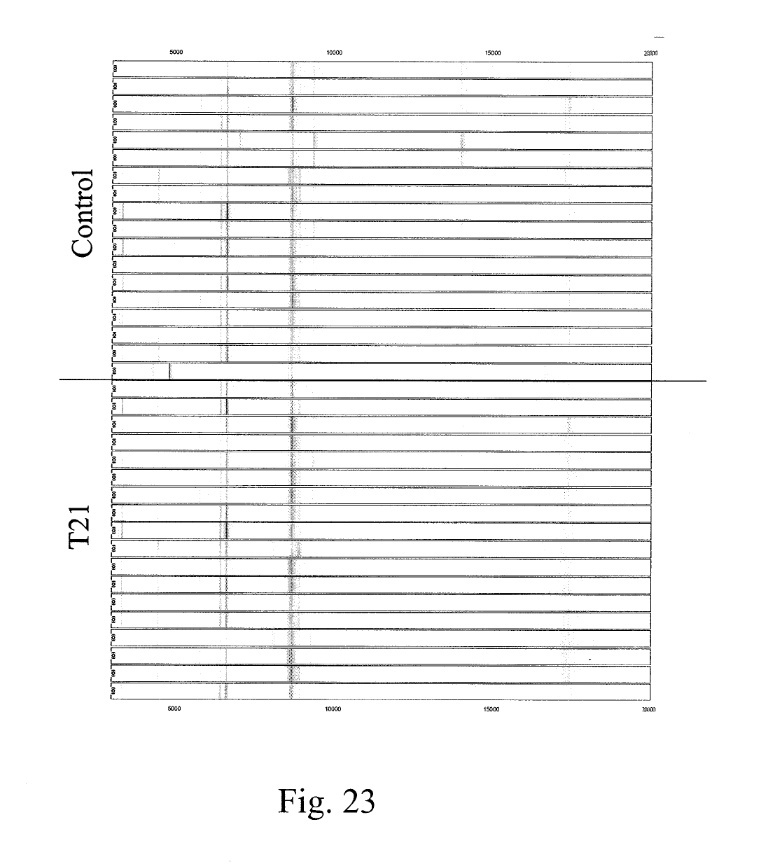

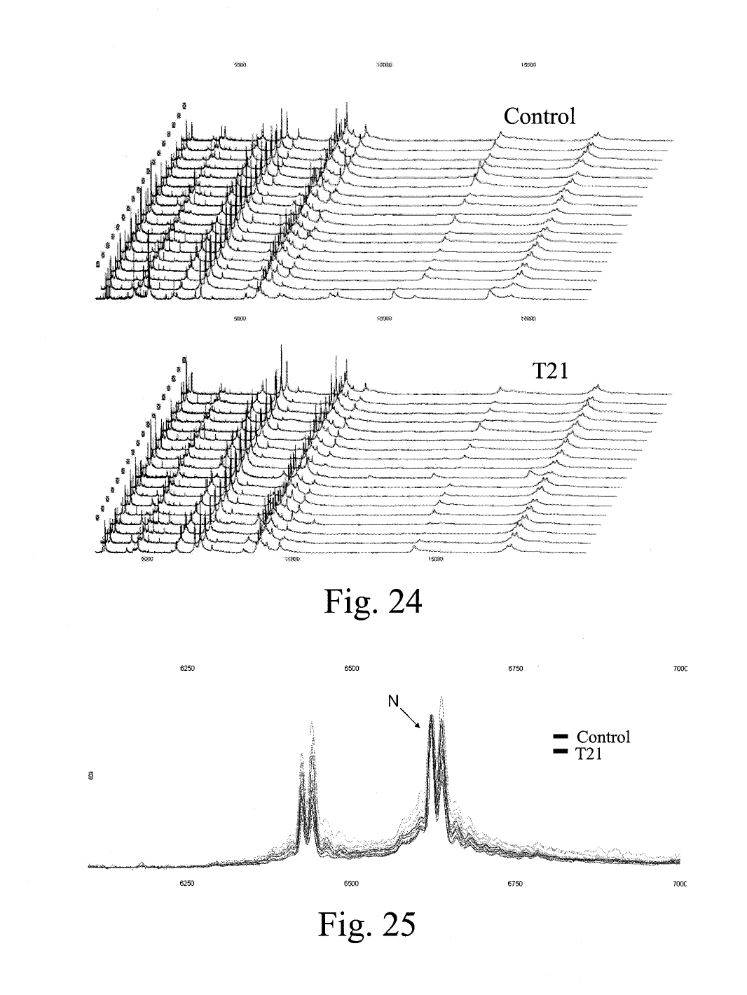

FIGS. 22-24 present the spectra over the 3000-2000 dalton spectral zone, respectively as overall views (spectra normalized and superimposed per clinical category) for the control, T21 and T18 groups, as pseudogel views for the control and T21 groups, and as shifted views for the same groups (study E6);

FIG. 25 shows the spectra centered on the 6500 zone, after normalization with respect to the intensity of the cluster MW6622 (studies E4 and E5). This figure shows the superimposition of the spectra for the control and T21 categories (study E6);

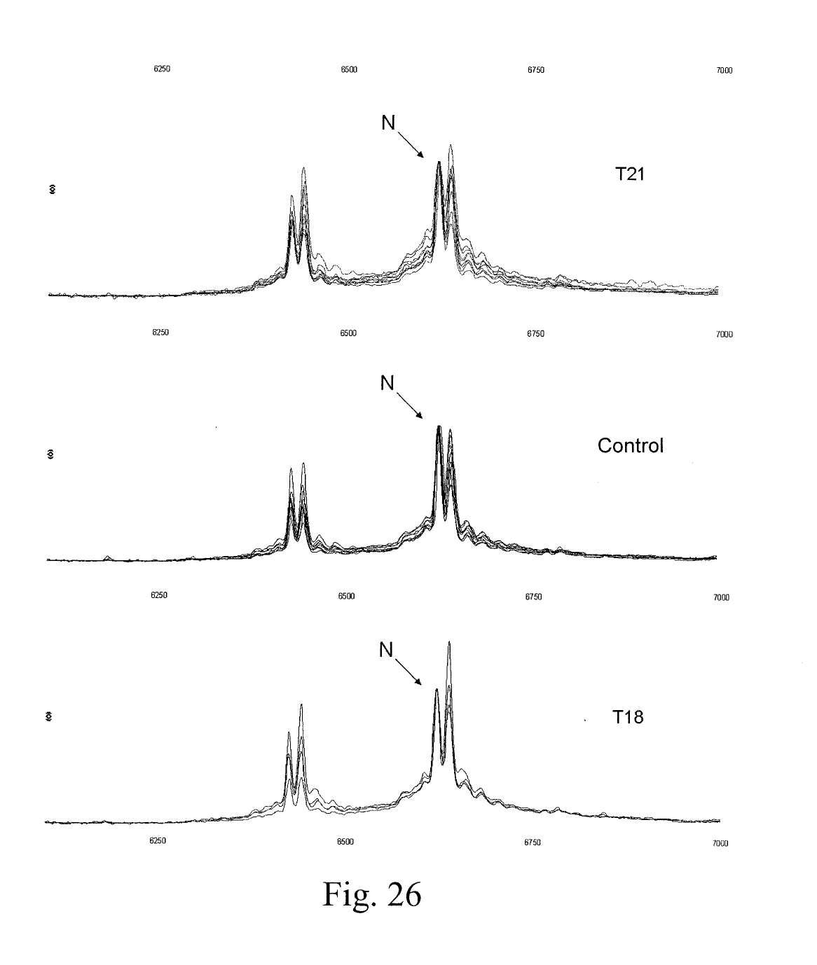

FIG. 26 shows the spectra centered on the 6500 zone after normalization on the MW6622 peak (spectra normalized and superimposed per clinical category), for the T21, control and T18 groups. In this figure, the peak referenced N corresponds to the MW6622 cluster and serves to normalize the spectra;

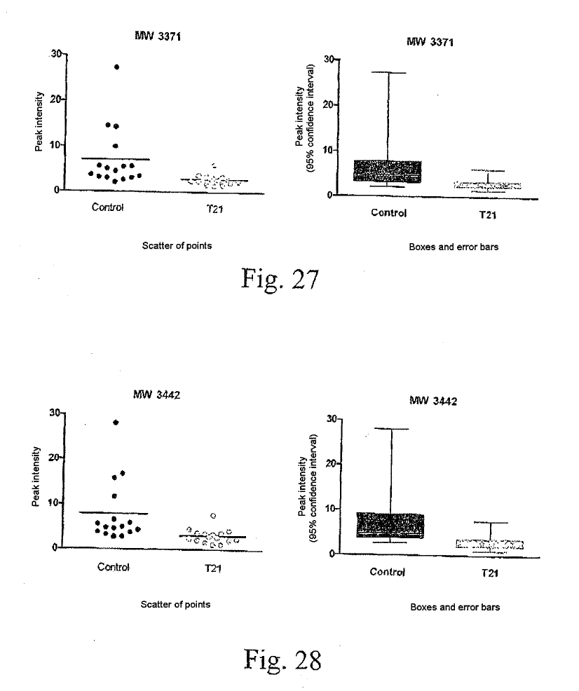

FIGS. 27 to 30 show examples of distribution statistics for the intensities of various clusters, for the control and T21 groups:

FIG. 27: peak (cluster) MW3371, anionic separating phase, any pH>4,

FIG. 28: peak MW3442, anionic separating phase, any pH>4,

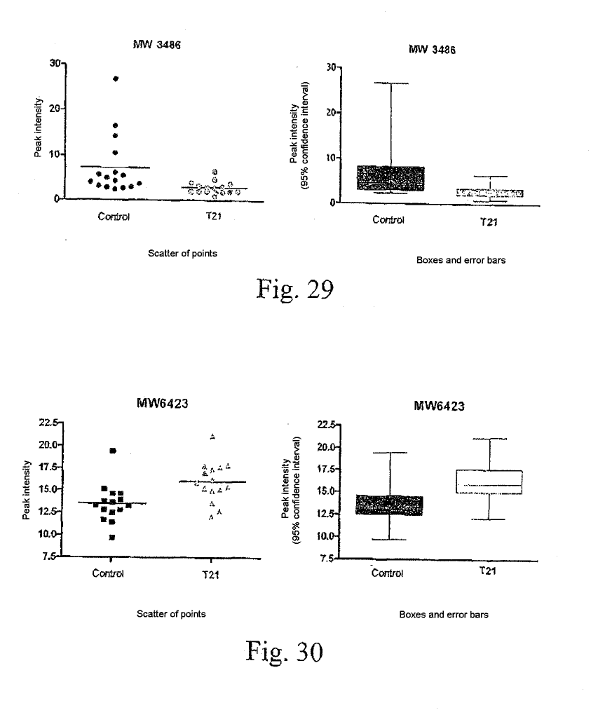

FIG. 29: peak MW3486, anionic separating phase, any pH>4,

FIG. 30: peak MW6423, hydrophobic separating phase, normalized with respect to the intensity of the peak MW6620;

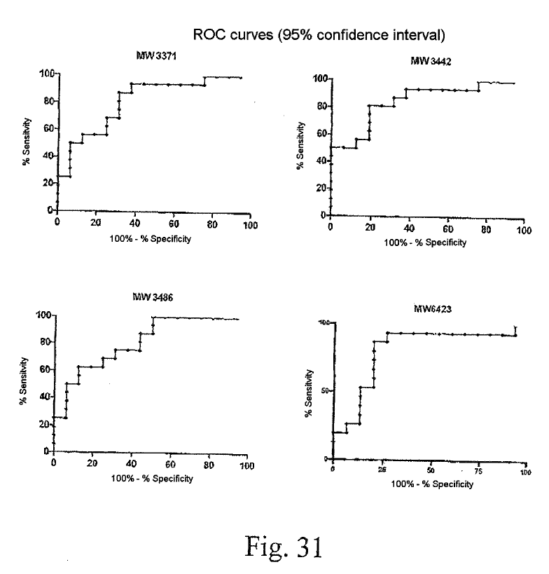

FIG. 31 shows the ROC curves for the markers of molecular weights 3371, 3442, 3486 and 6423 Da;

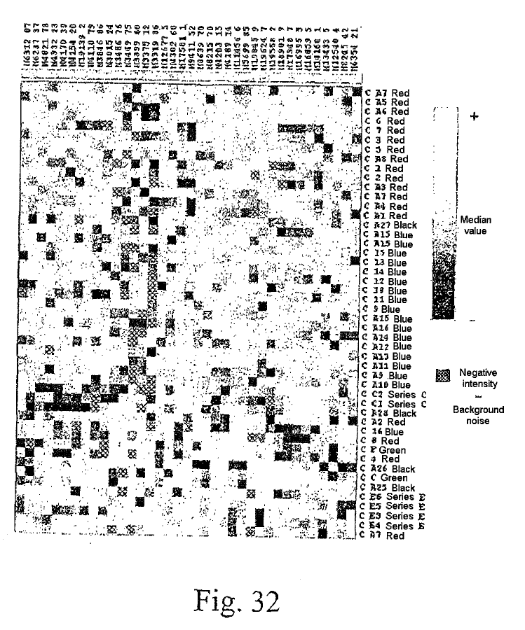

FIG. 32 shows the result of a construction with the Genecluster Version 1999 (Eisen M.) and Treeview Version 1.60 (Eisen M.) programs (hierarchical classification on the set of data of study E6 (IC 95%), anionic surface Q10);

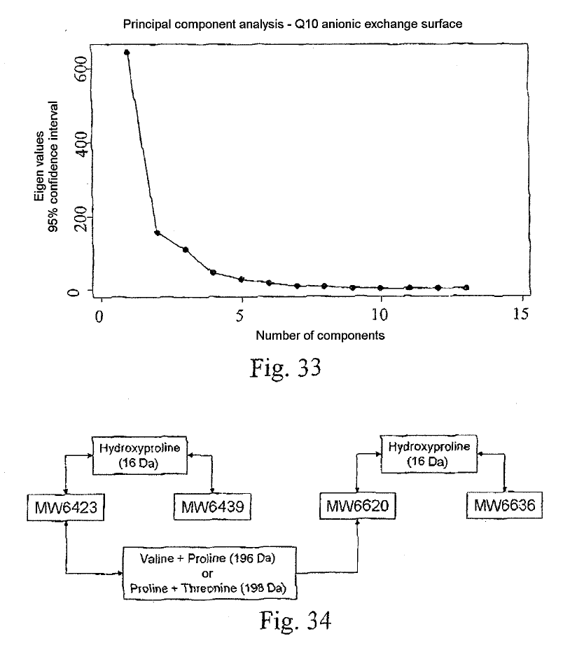

FIG. 33 illustrates the main-component analysis for the defensins (Q10 anion exchange surface);

FIG. 34 illustrates the procedure for identifying the peaks (clusters) MW6423, MW6439, MW6620 and MW6636;

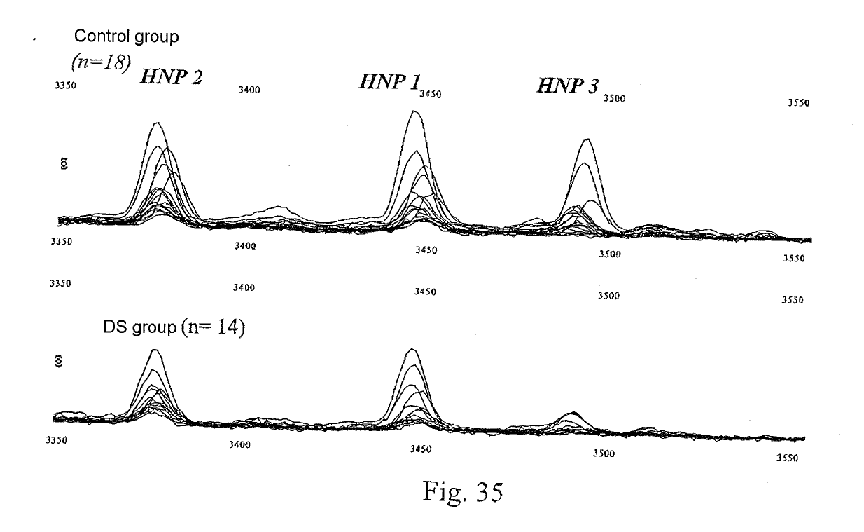

FIG. 35 shows mass spectra acquired under conditions comprising Q10, pH 4, crude serum, −80° C., for the zone corresponding to the HNPs, for a control group and a group of sera from women carrying fetuses carrying trisomie 21;

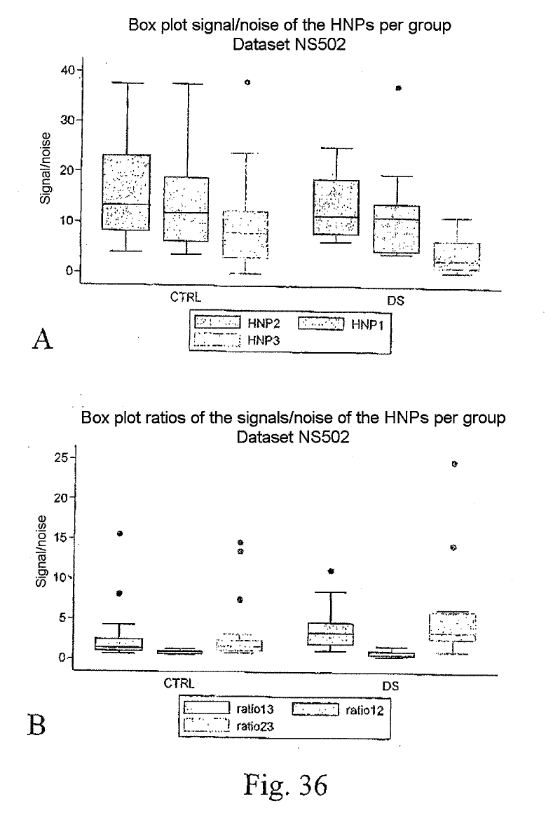

FIG. 36 presents the statistical data relating to the HNPs, as signal-to-noise, in the form of box plots. A: signal-to-noise of each peak. B: signal/noise longitudinal ratios;

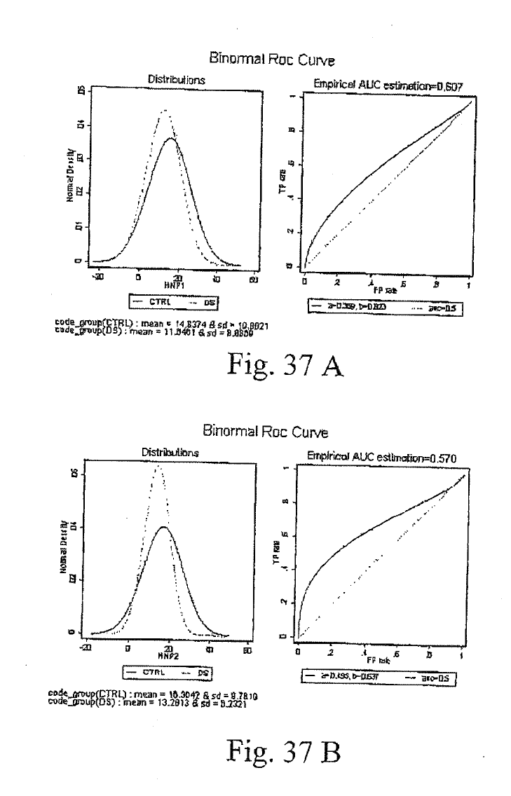

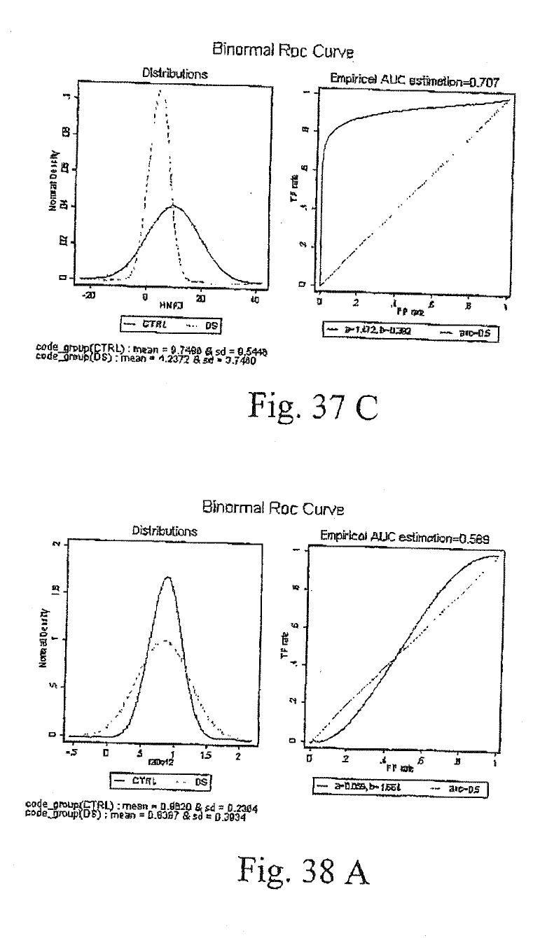

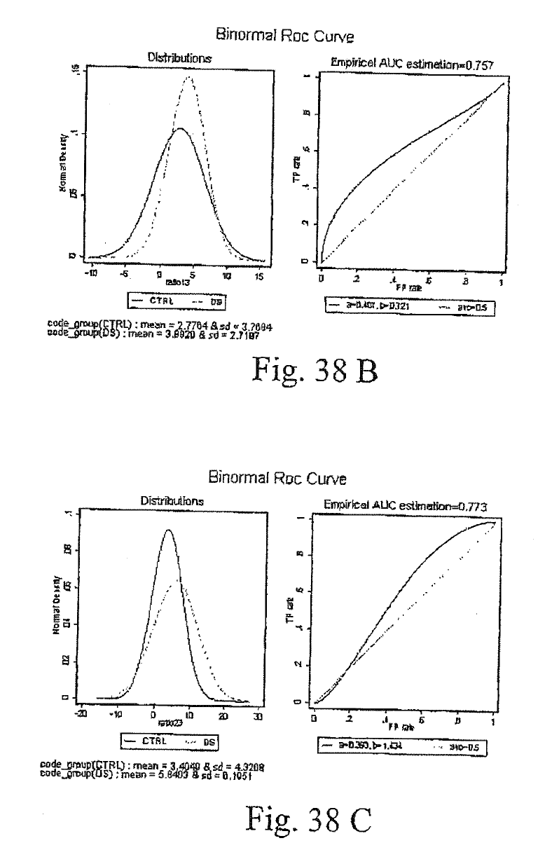

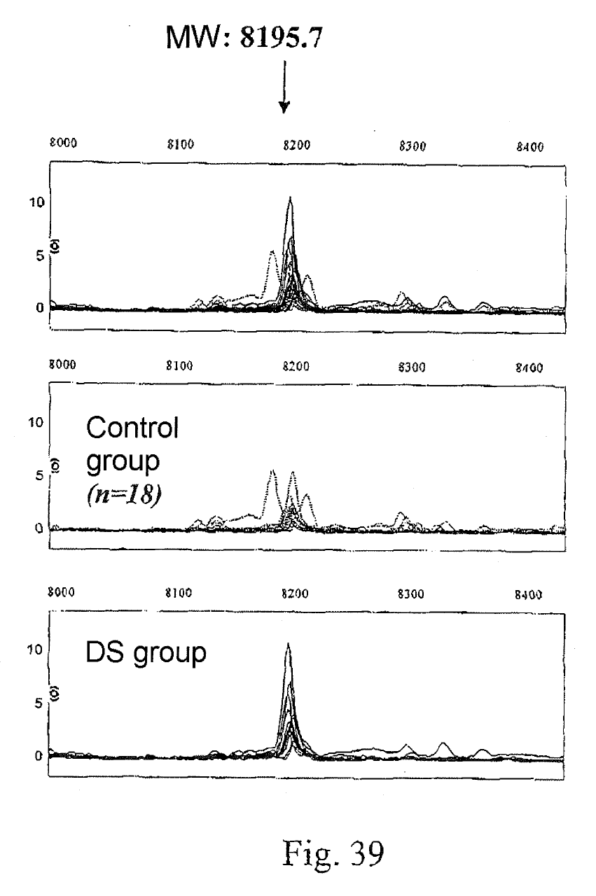

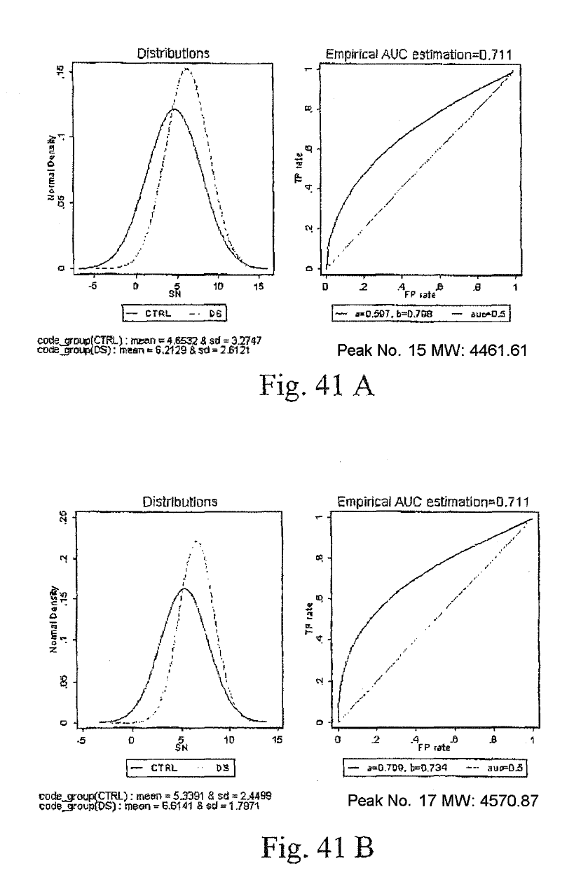

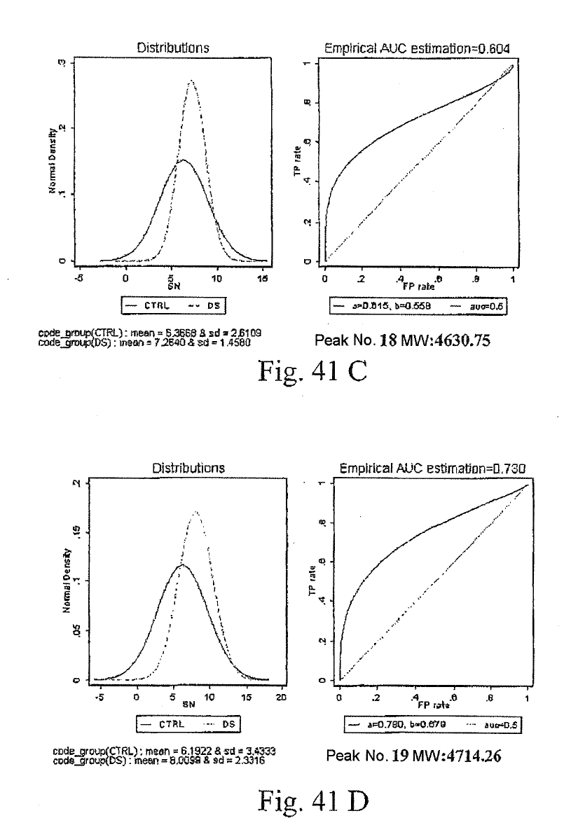

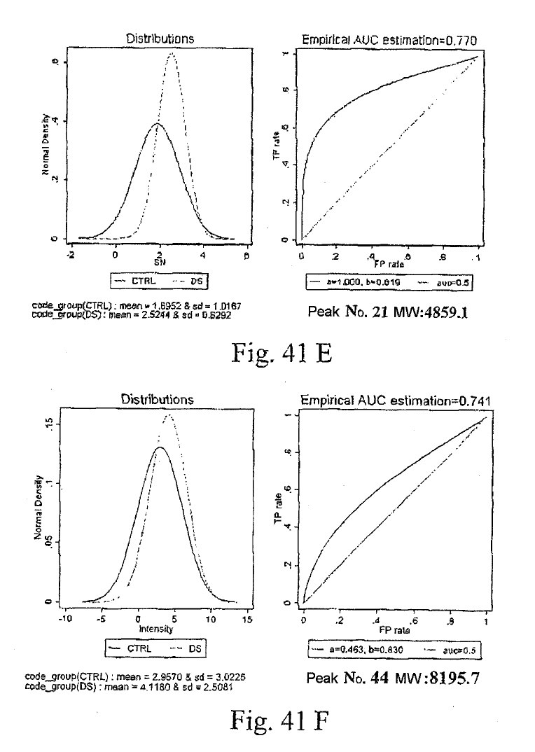

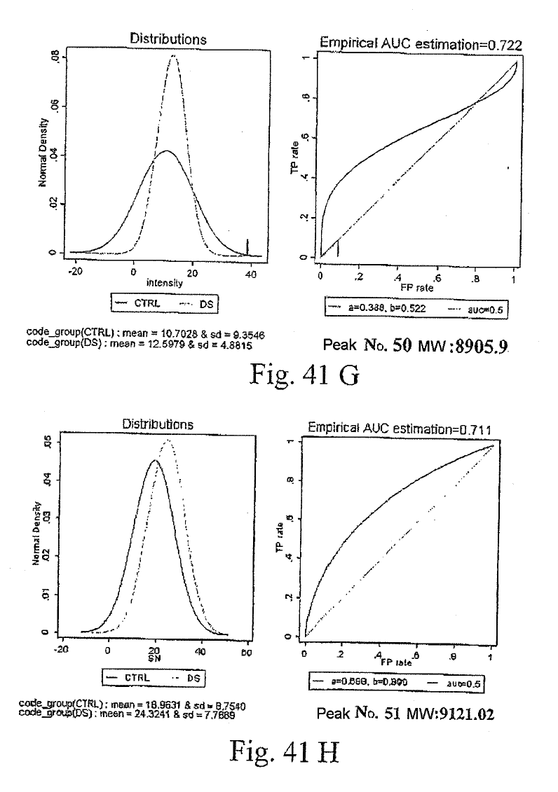

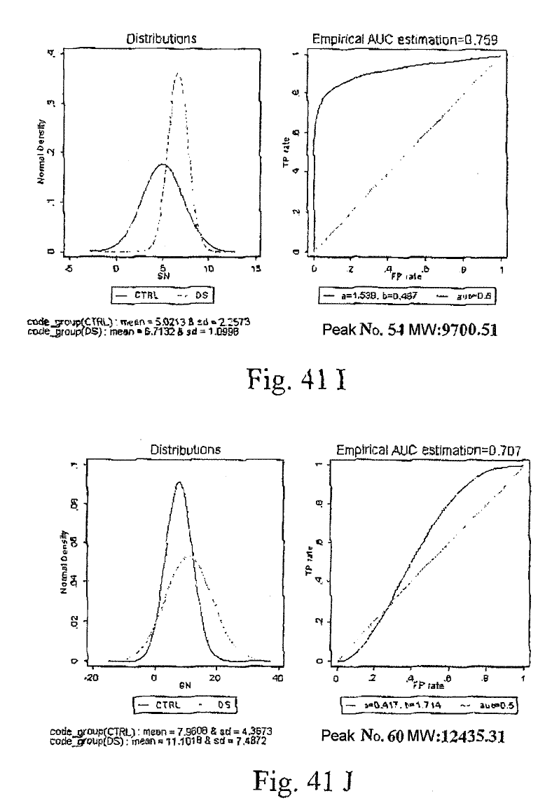

FIGS. 37 and 38 present the binormal ROC curves and distributions corresponding to each HNP (FIG. 37A: HNP1; 37B: HNP2; 37C: HNP3) and to each longitudinal ratio between two HNPs (FIG. 38A: 1/2; 38B: 1/3; 38C: 2/3), all as signal/noise. For these figures, and also FIGS. 41, 49 and 50, the number of the peak and its molecular weight (MW) are indicated, and the following terms are used: empirical AUC estimation, which is a nonparametric estimation calculated on the continuous data of the signal-to-noise ratio; TP rate=true positive rate; FP rate=false positive rate; normal density; CTRL=control; DS=Down syndrome; mean; sd=standard deviation;

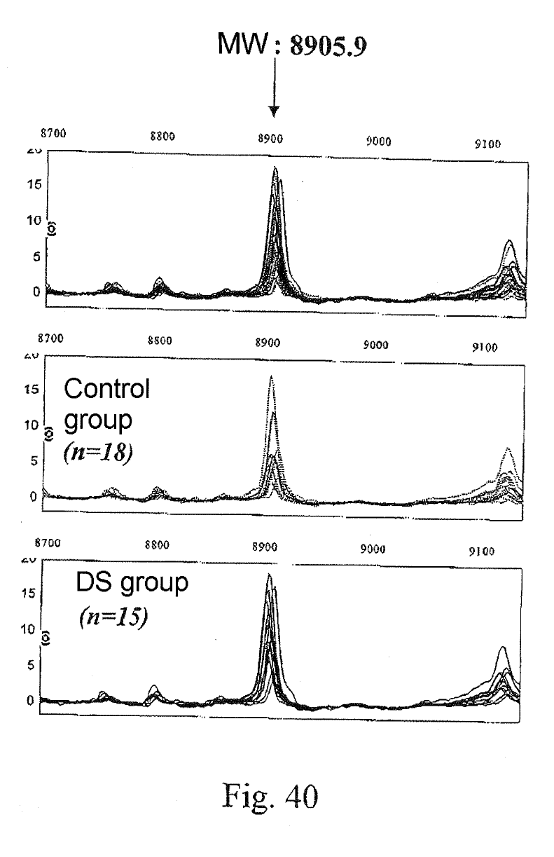

FIGS. 39 and 40 show mass spectra acquired under the same conditions as FIG. 35, centered on different zones;

FIG. 41 presents the binormal ROC curves and distributions associated with the peaks other than the HNPs identified as being discriminating under the conditions of Q10, pH 4, crude serum, −80° C.;

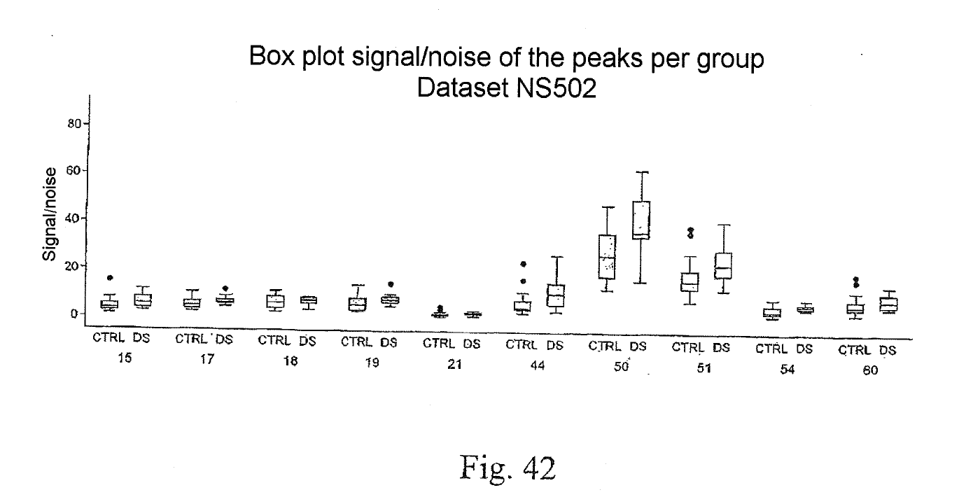

FIG. 42 summarizes the data (as signal/noise) of the relevant peaks (other than the HNPs) of the NS502 study, in the form of box plots. The isolated points represent the abnormal (aberrant) values, which depart from the confidence interval;

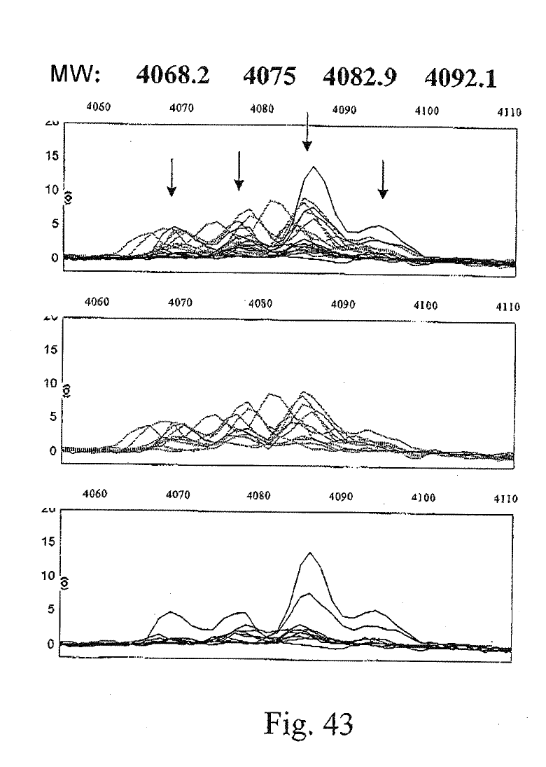

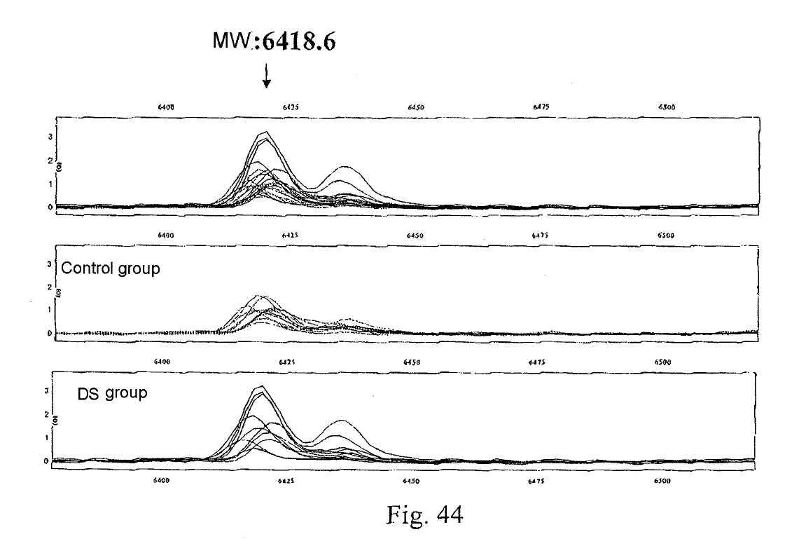

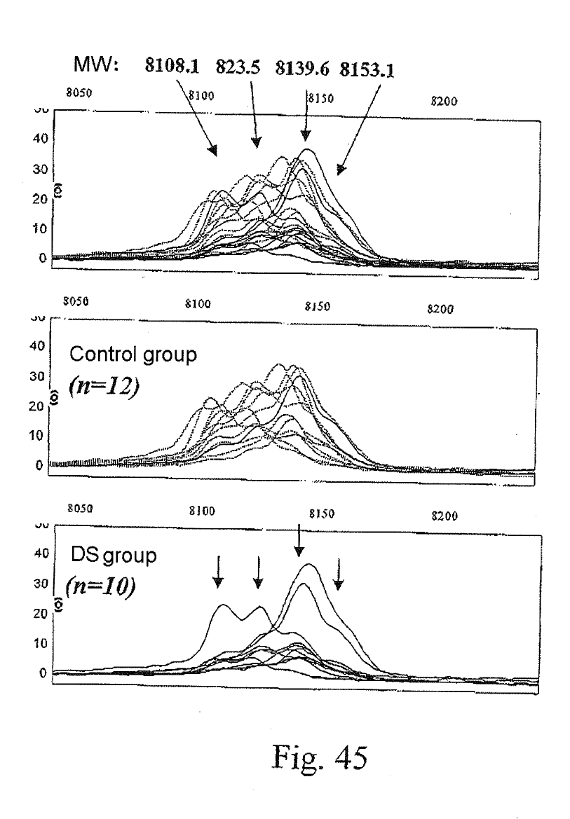

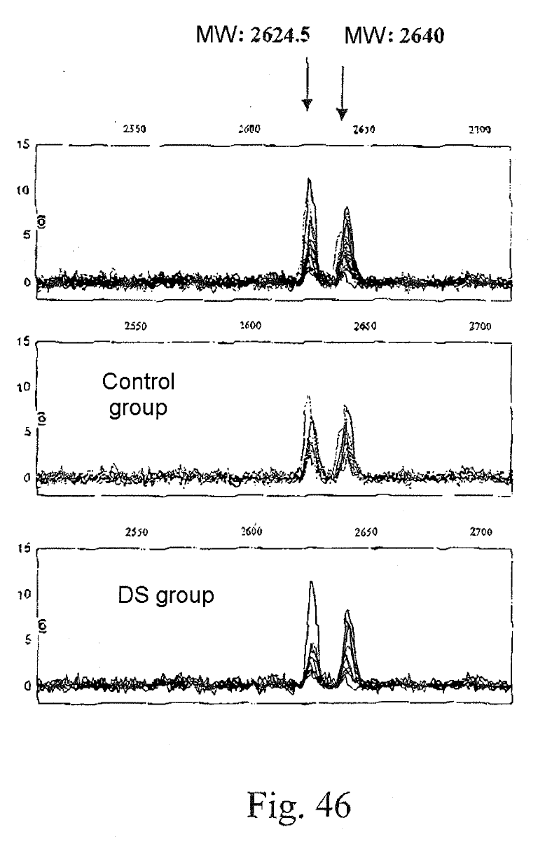

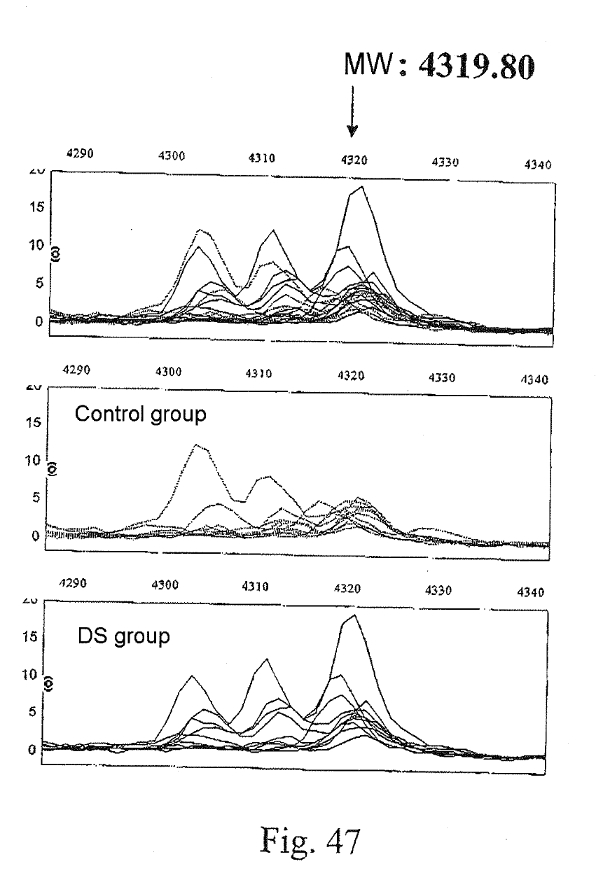

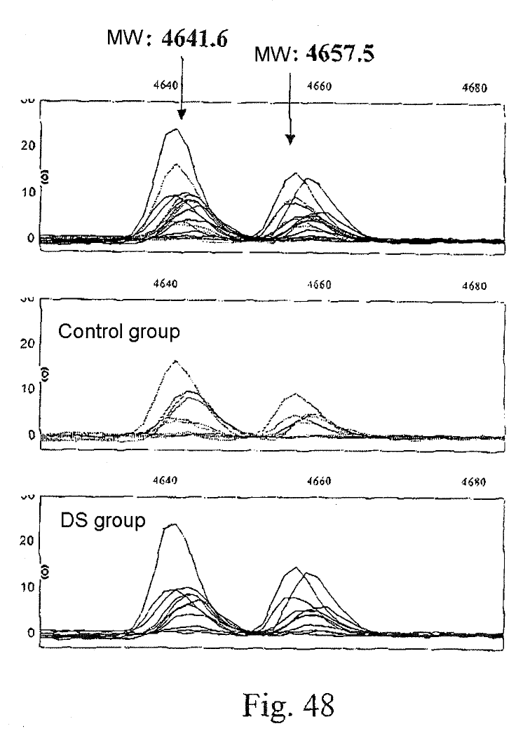

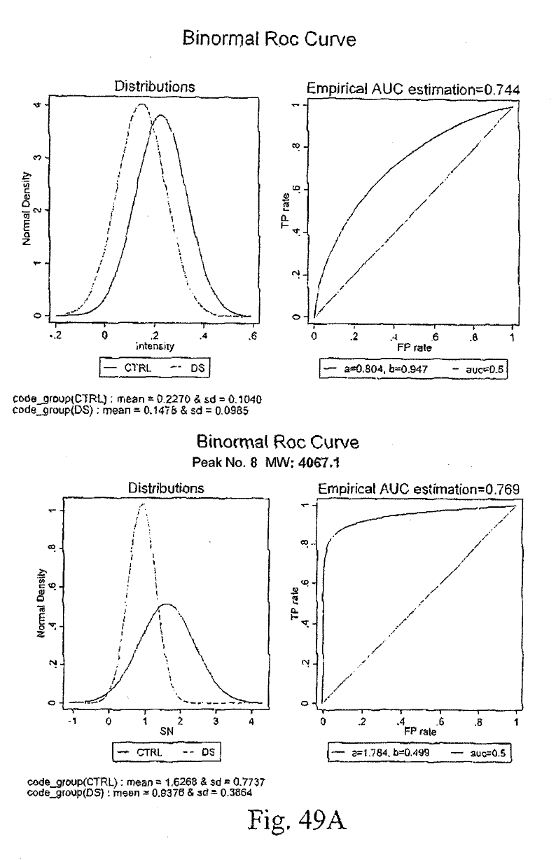

FIGS. 43 to 48 present spectra acquired under conditions of purification on H50, for certain zones identified as being relevant in the NS526c and NS526d studies;

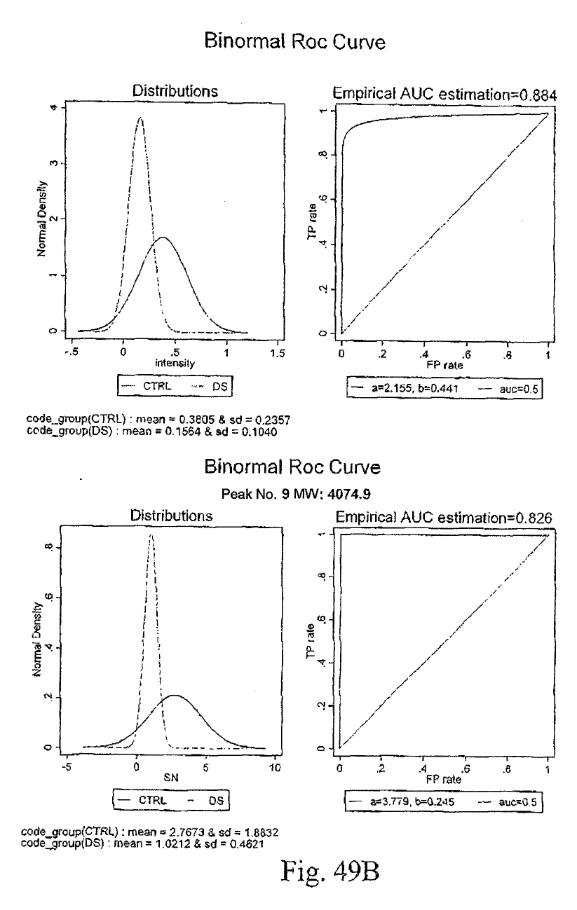

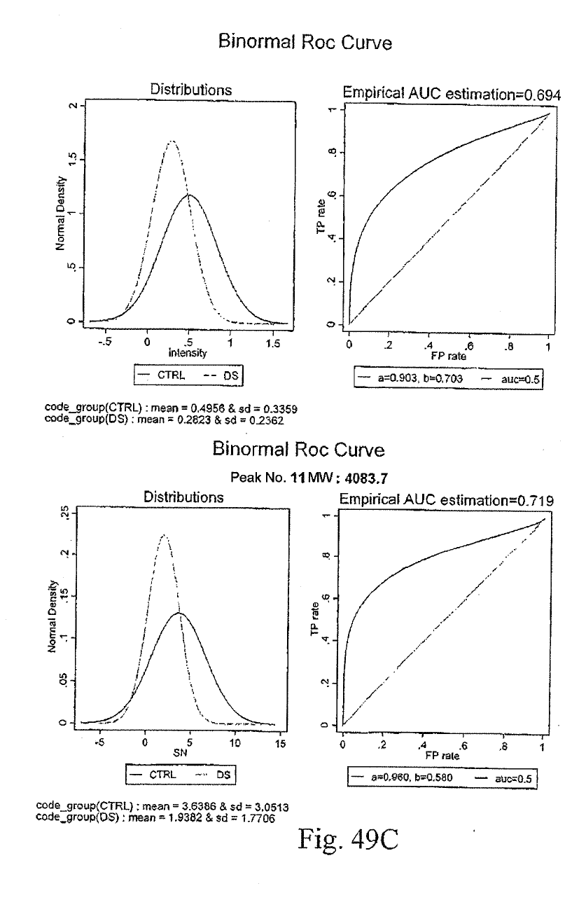

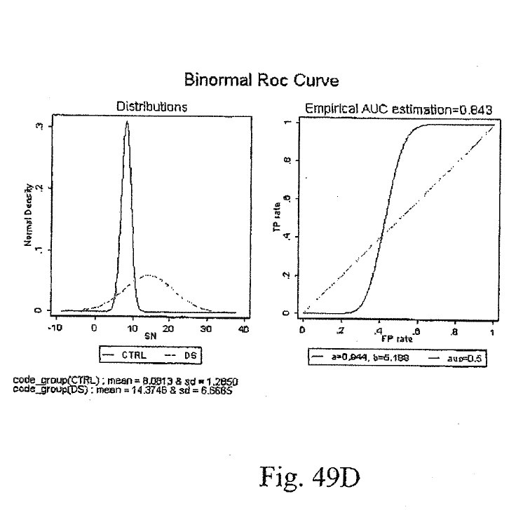

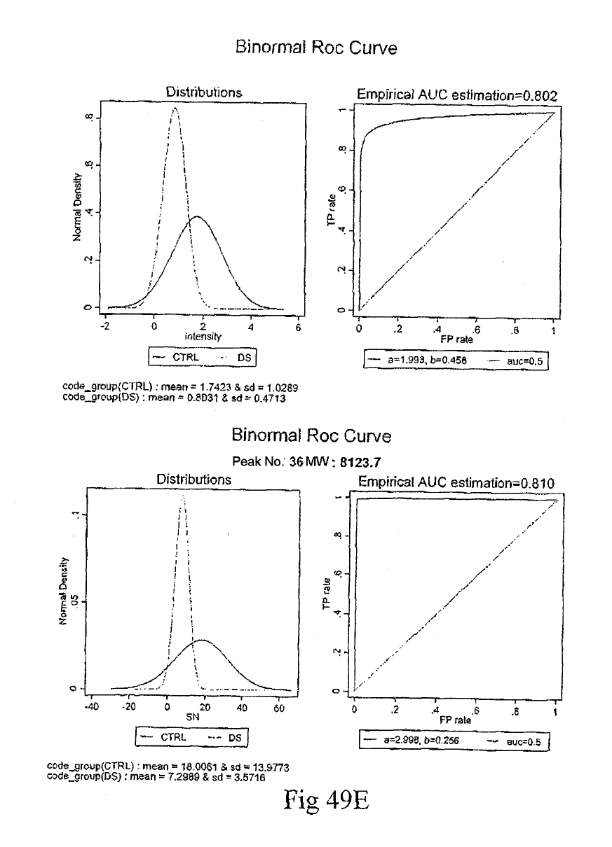

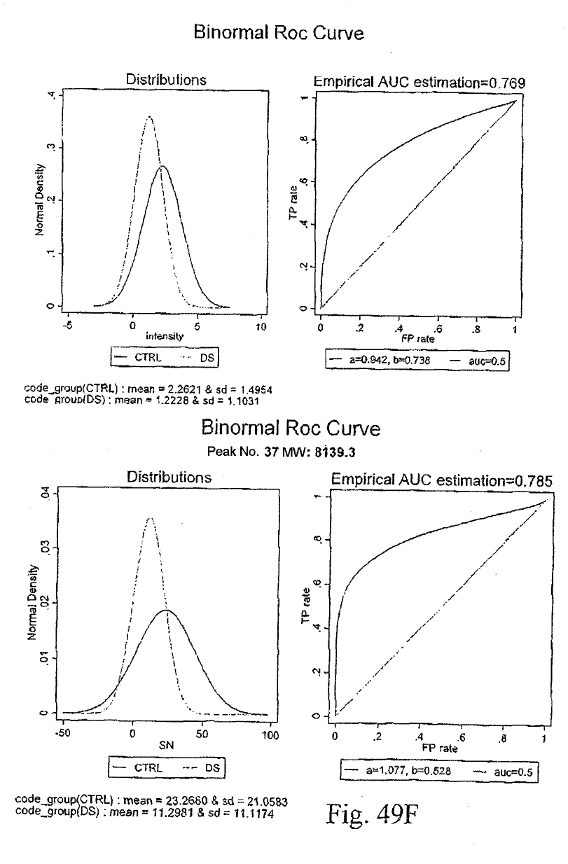

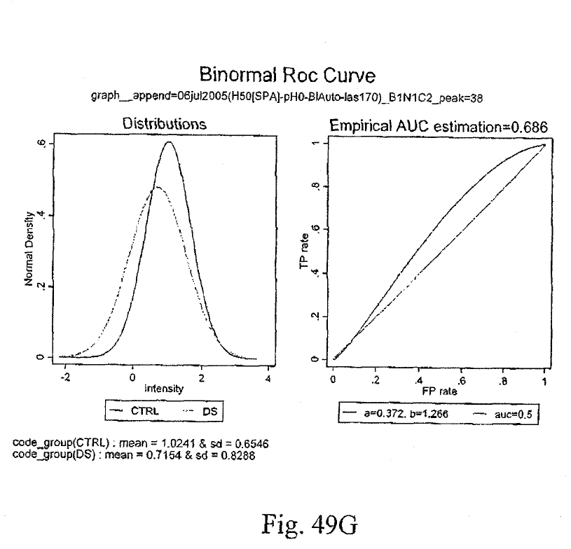

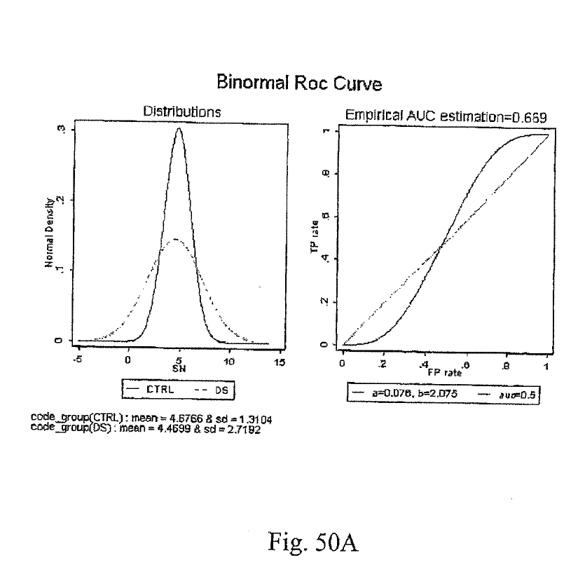

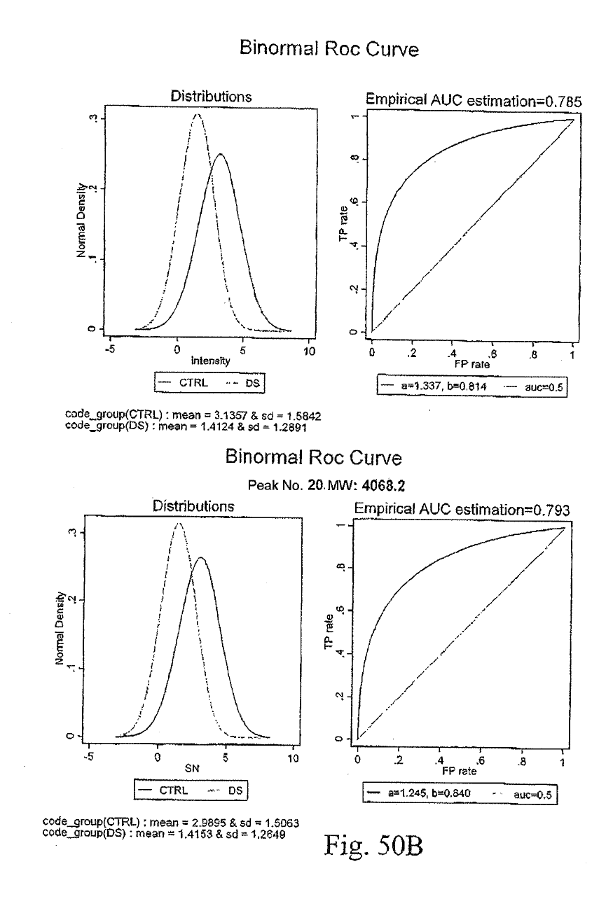

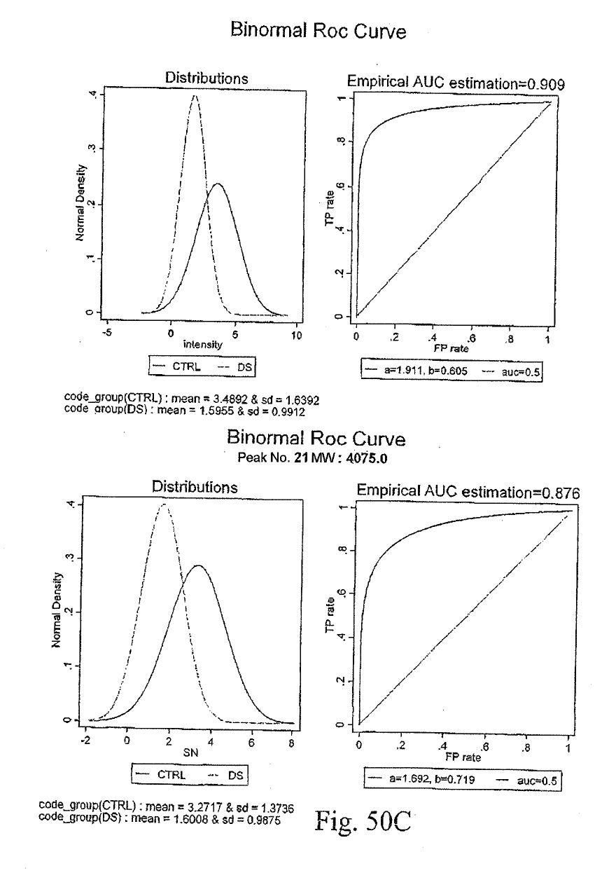

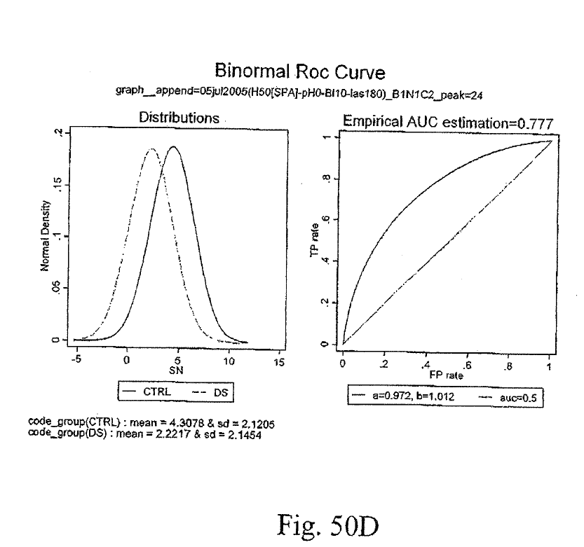

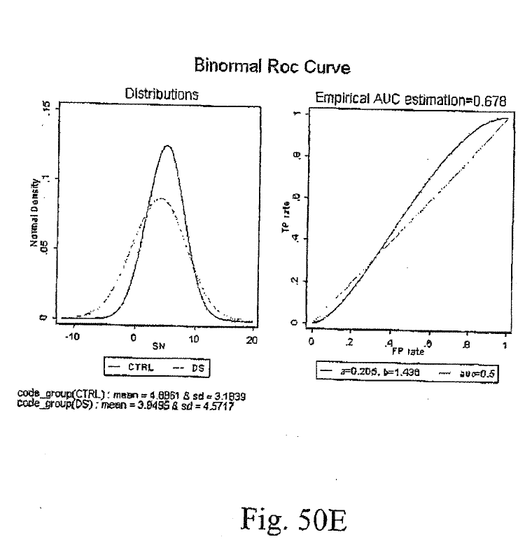

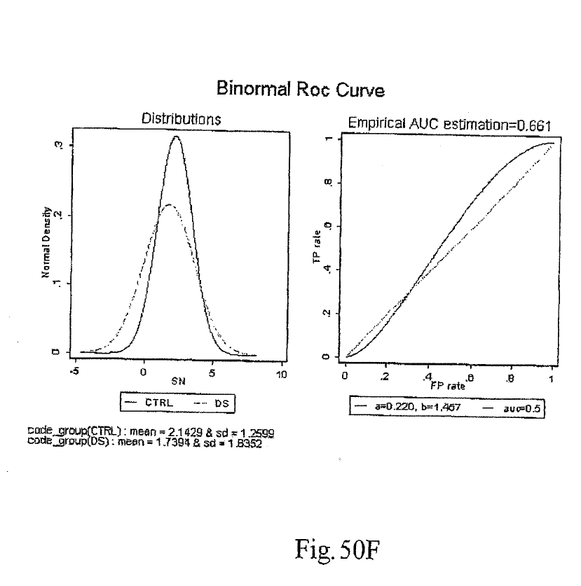

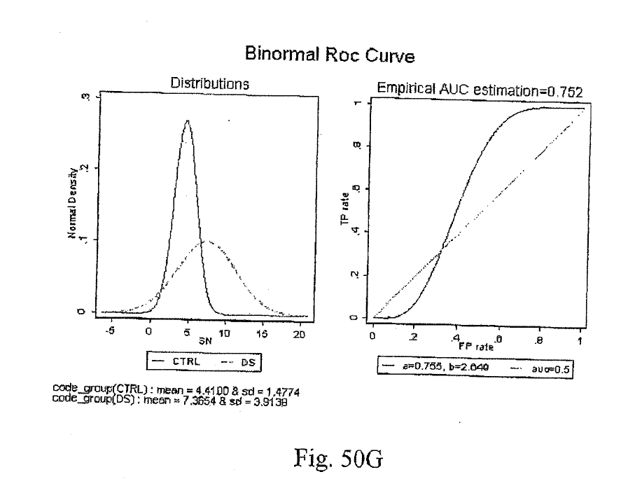

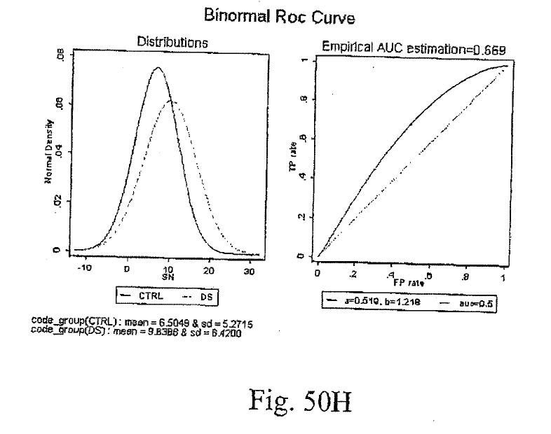

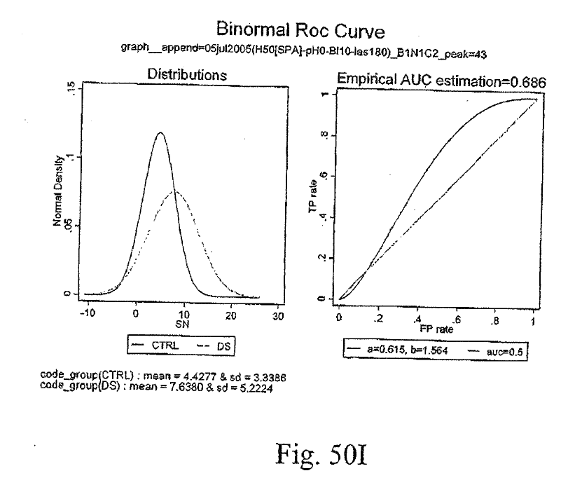

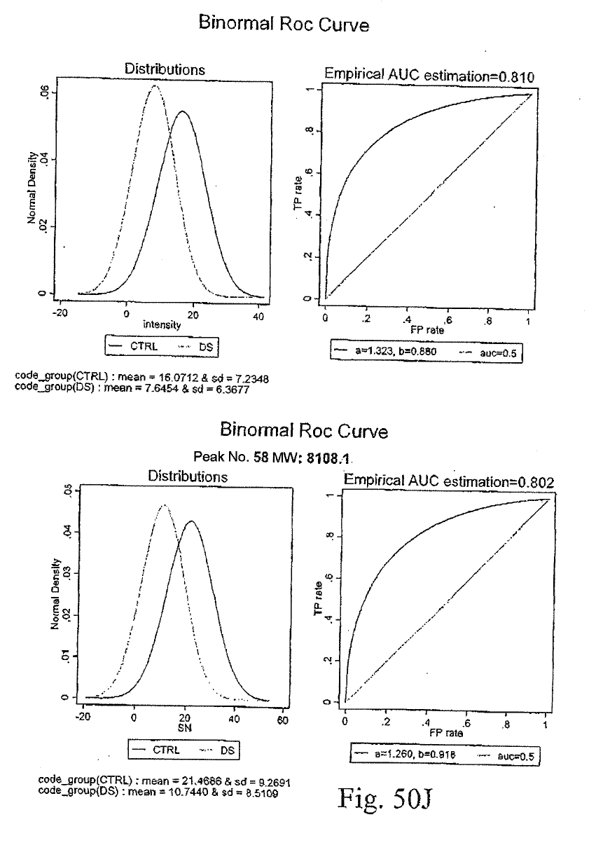

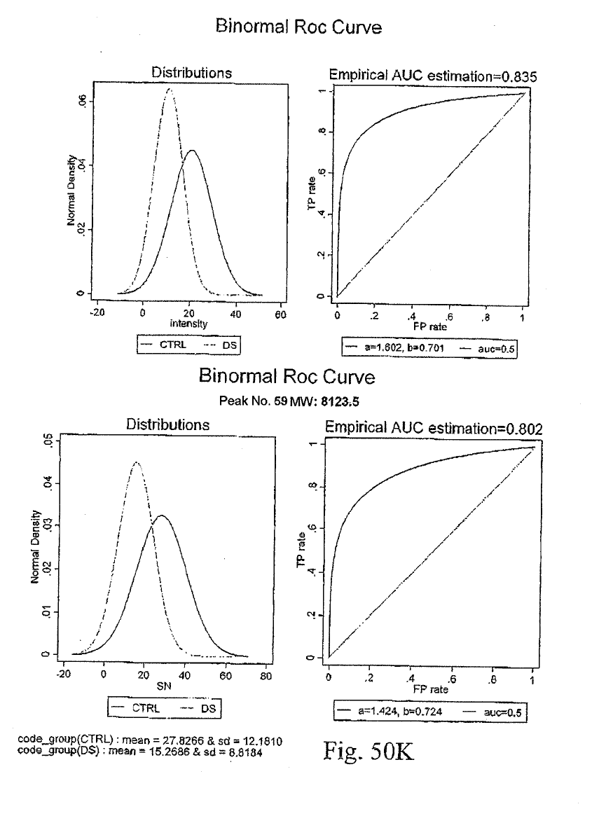

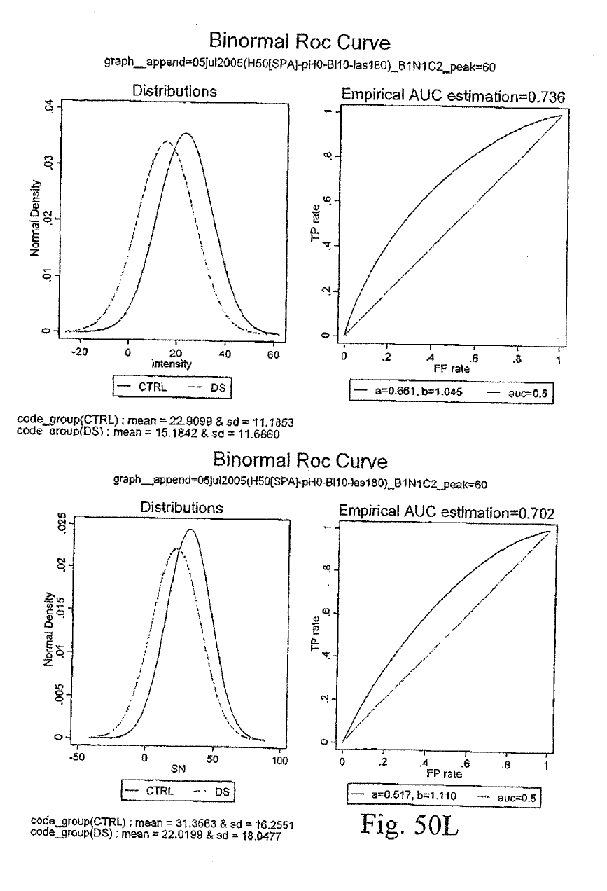

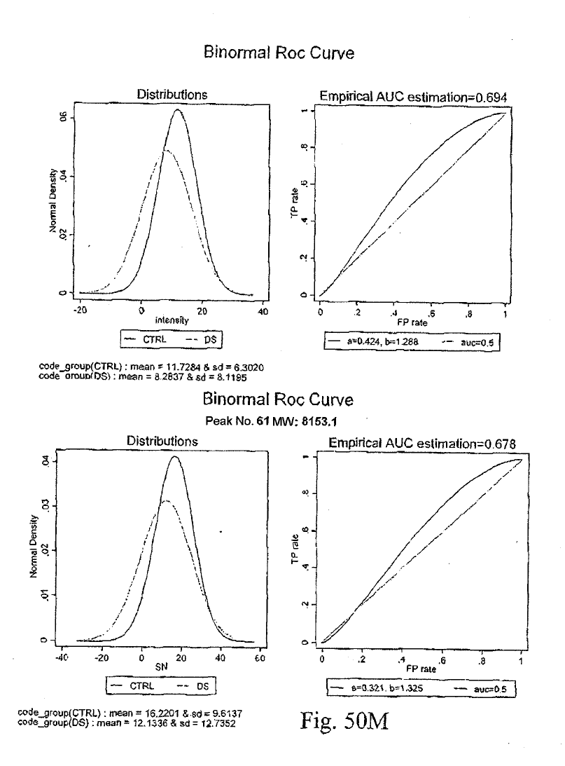

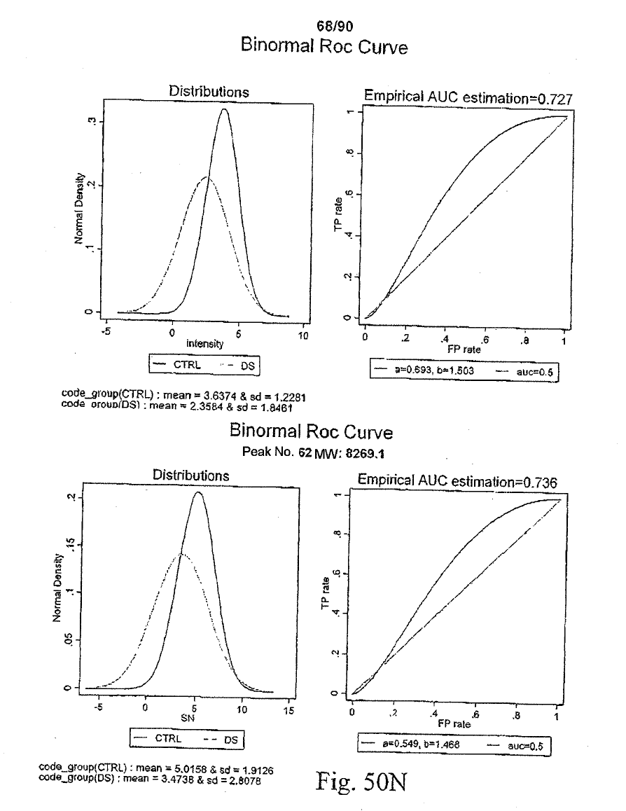

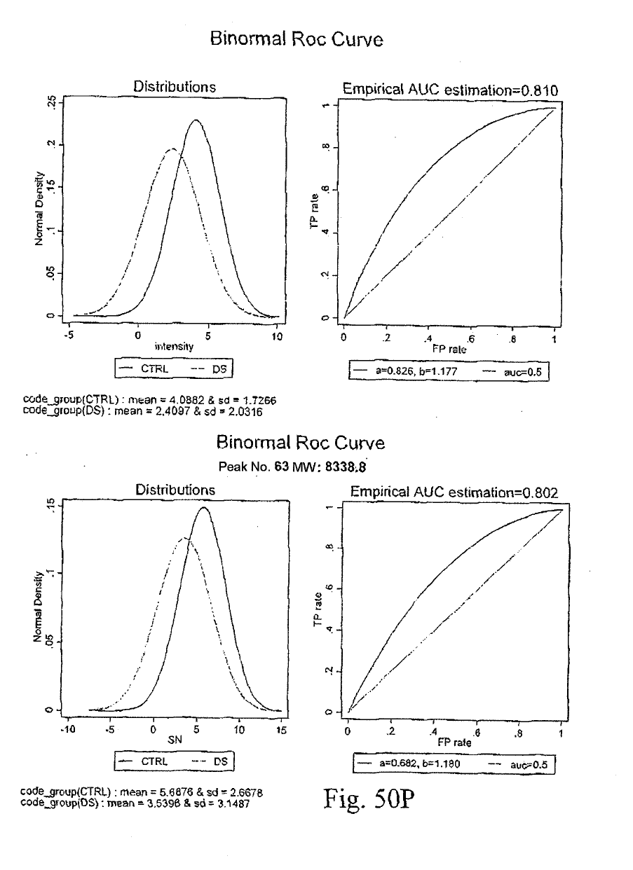

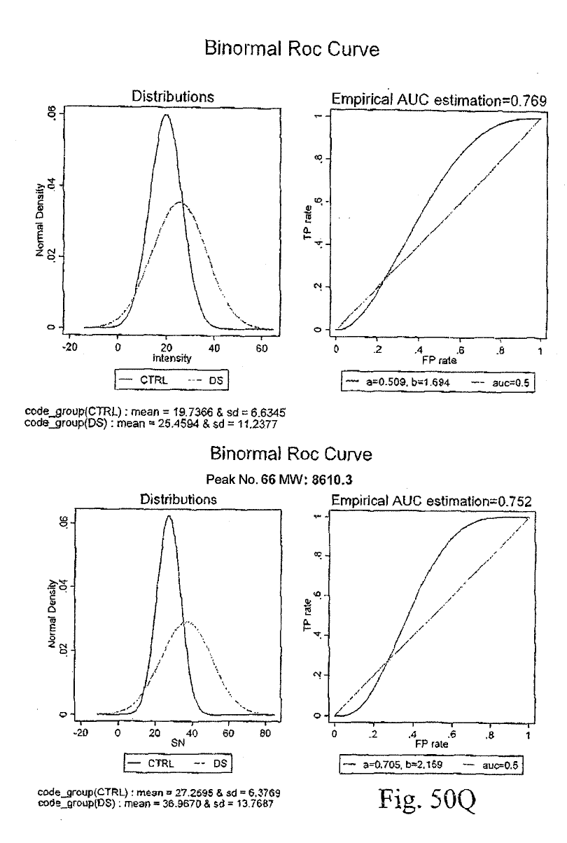

FIGS. 49 and 50 present the data relating to each peak (of the NS526c study and of the NS526d study, respectively), in the form of distribution curves and of ROC curves;

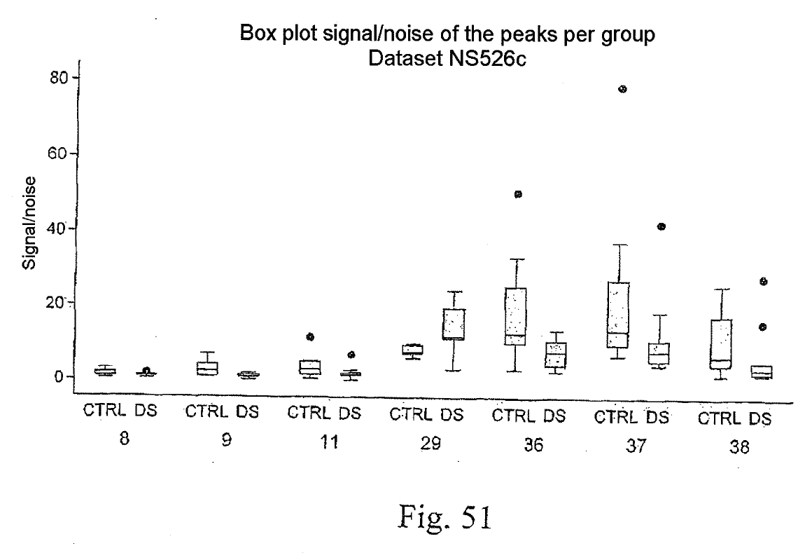

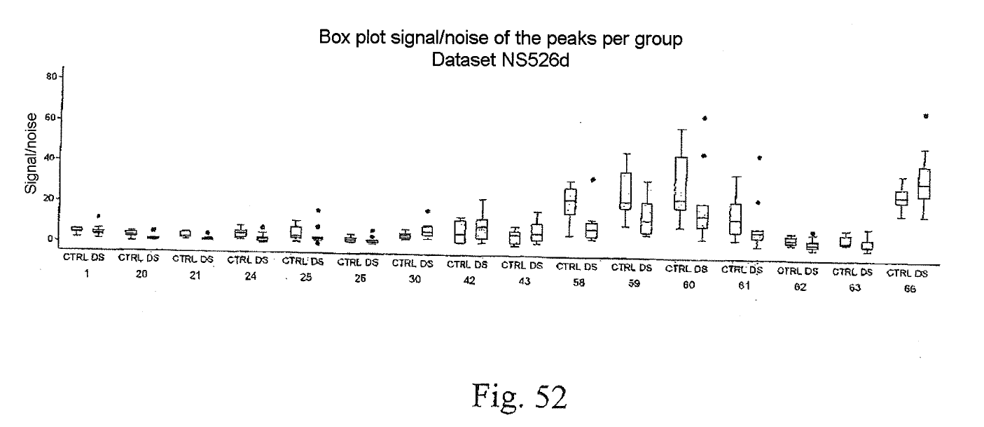

FIGS. 51 and 52 show the results, in the form of box plots, for the markers identified in the NS526c and NS526d studies;

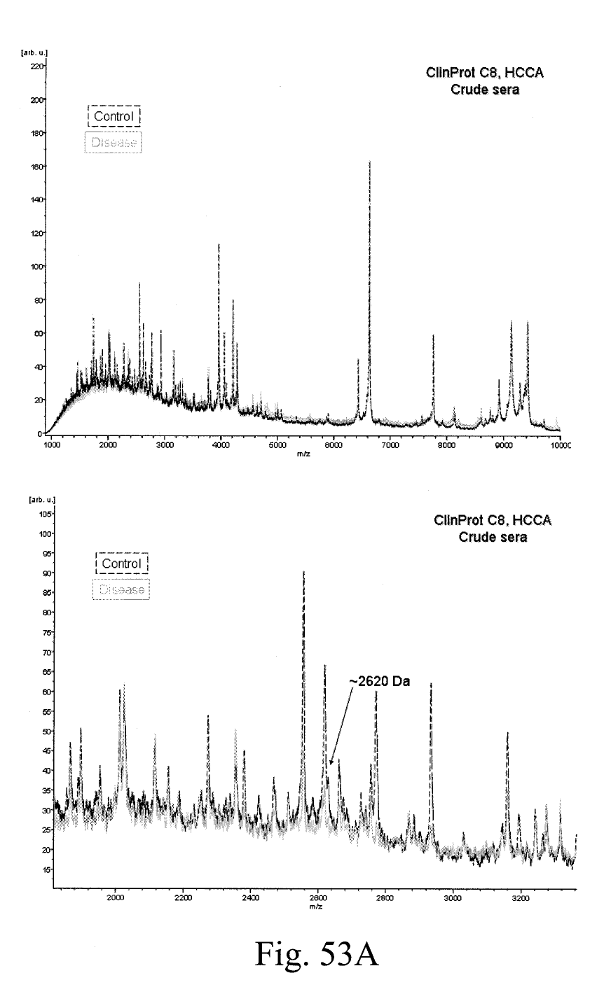

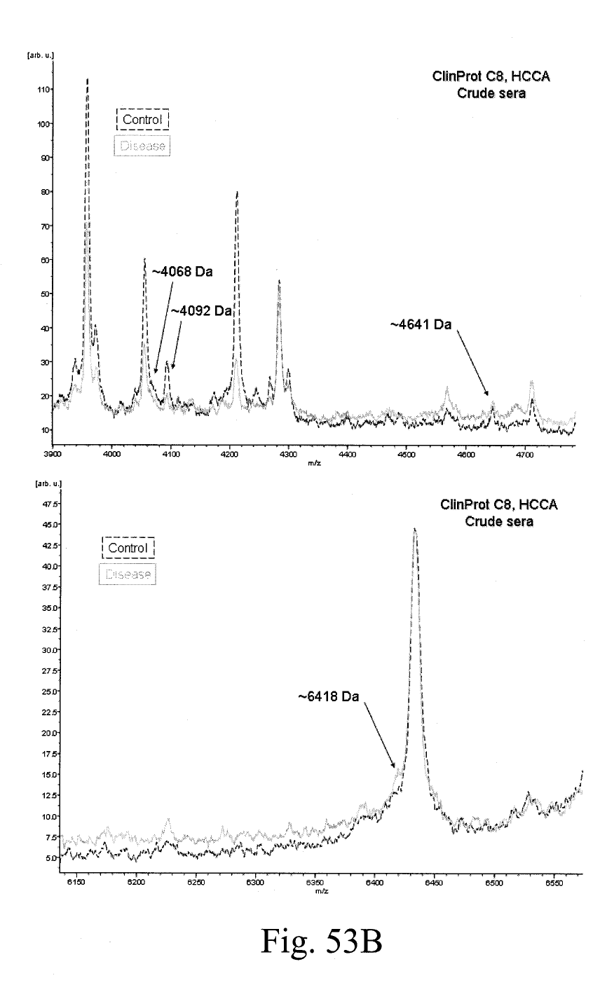

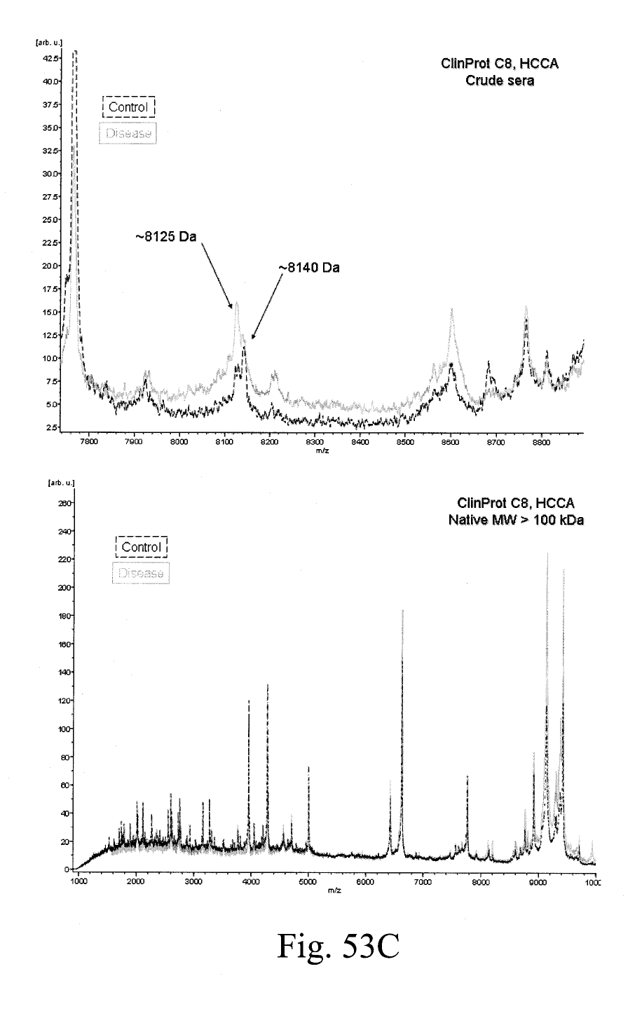

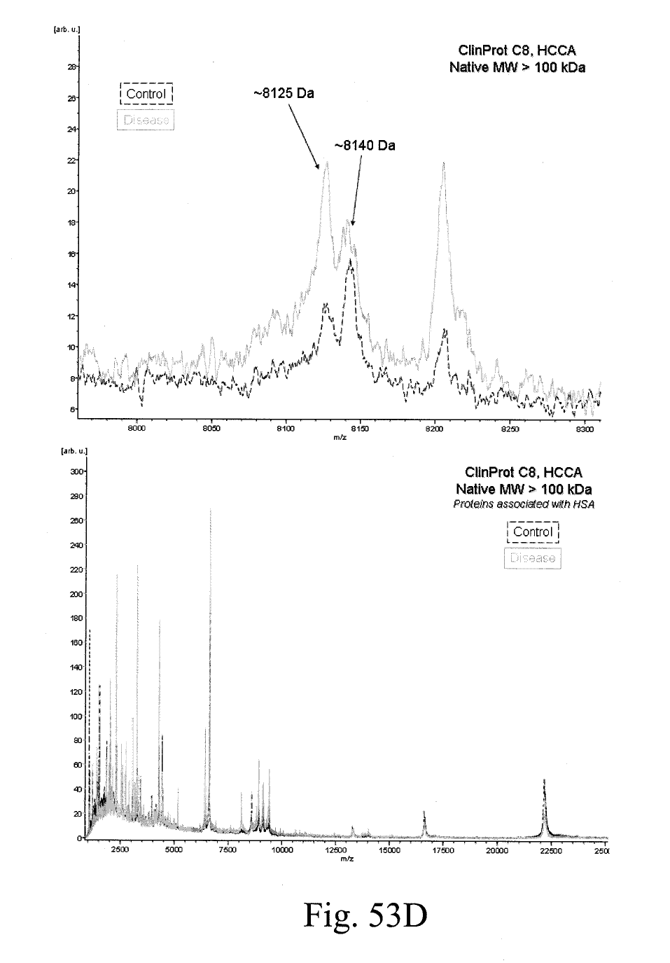

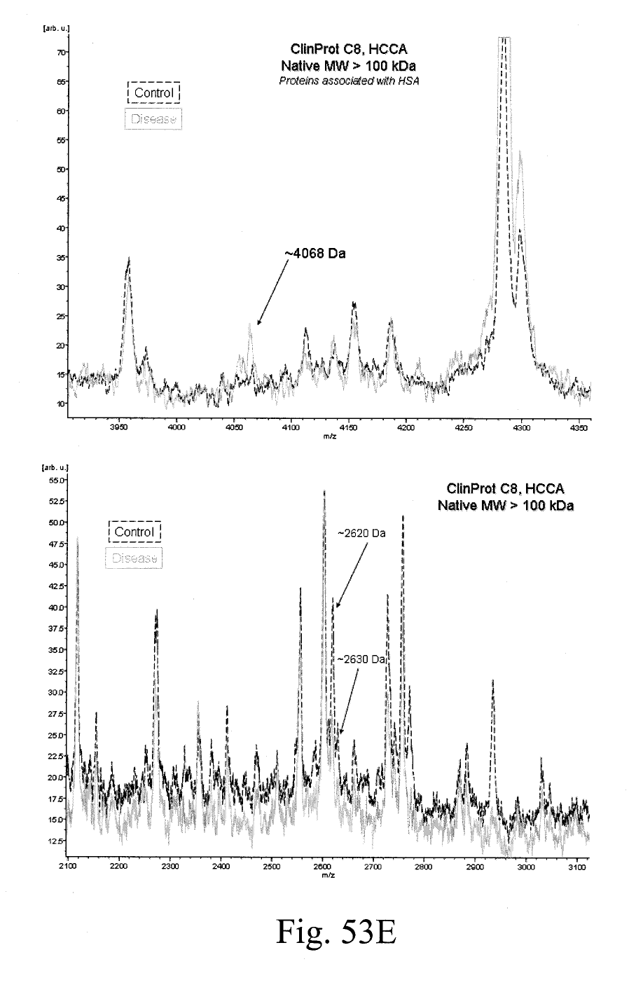

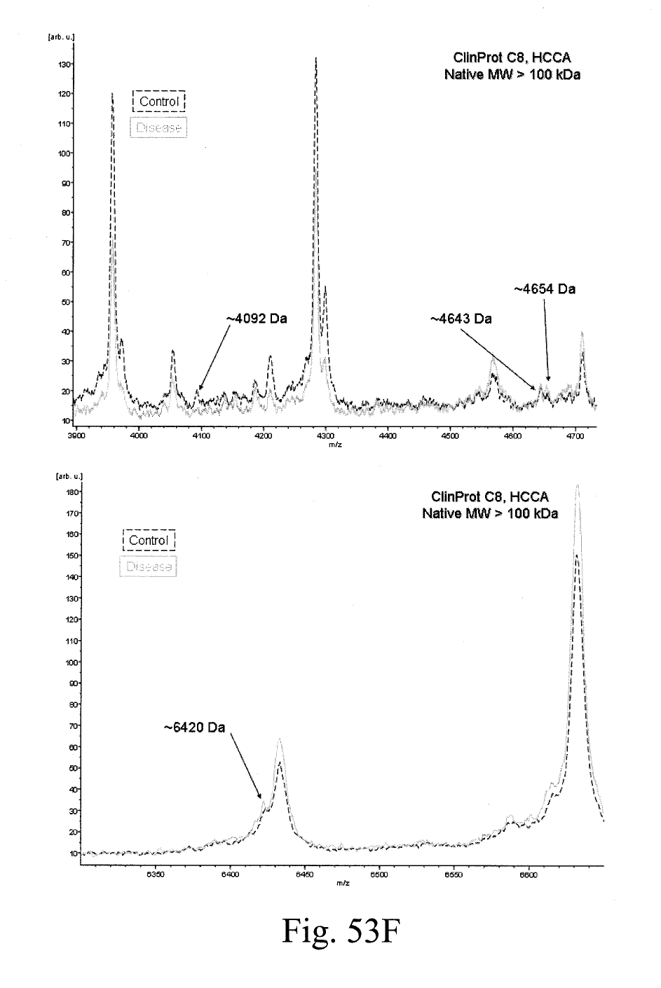

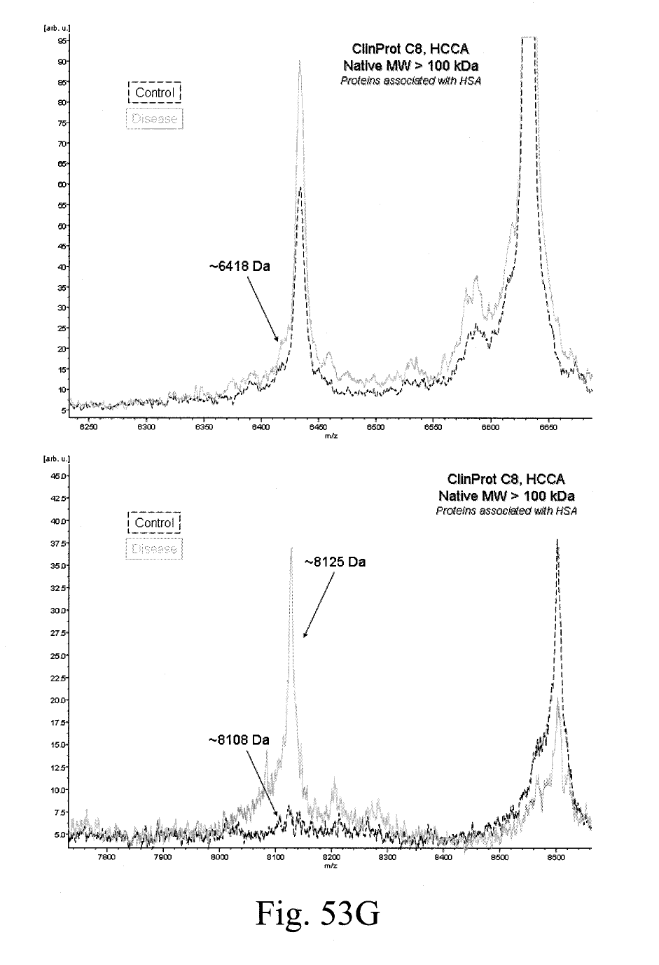

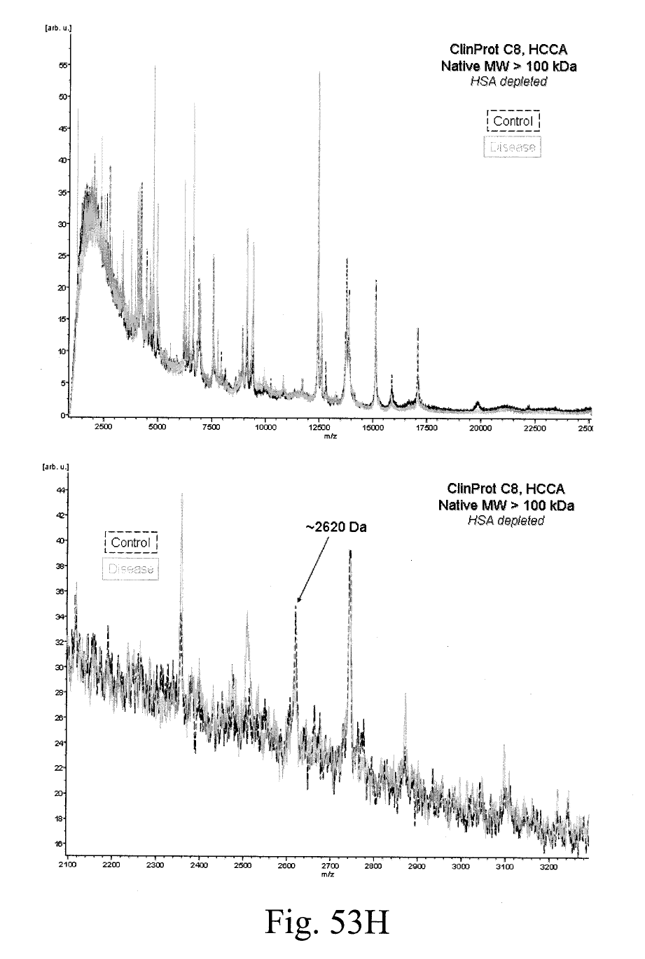

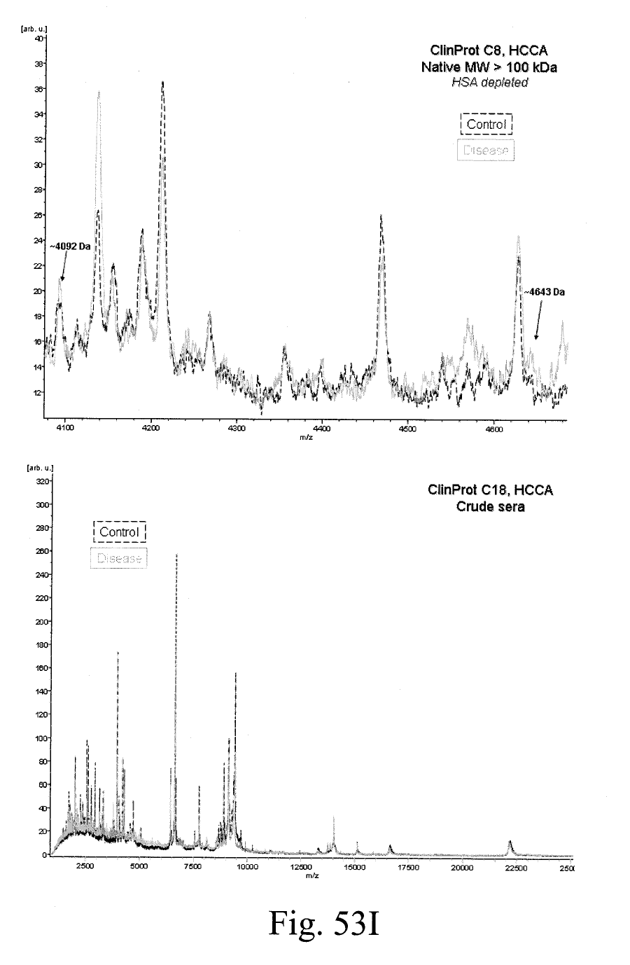

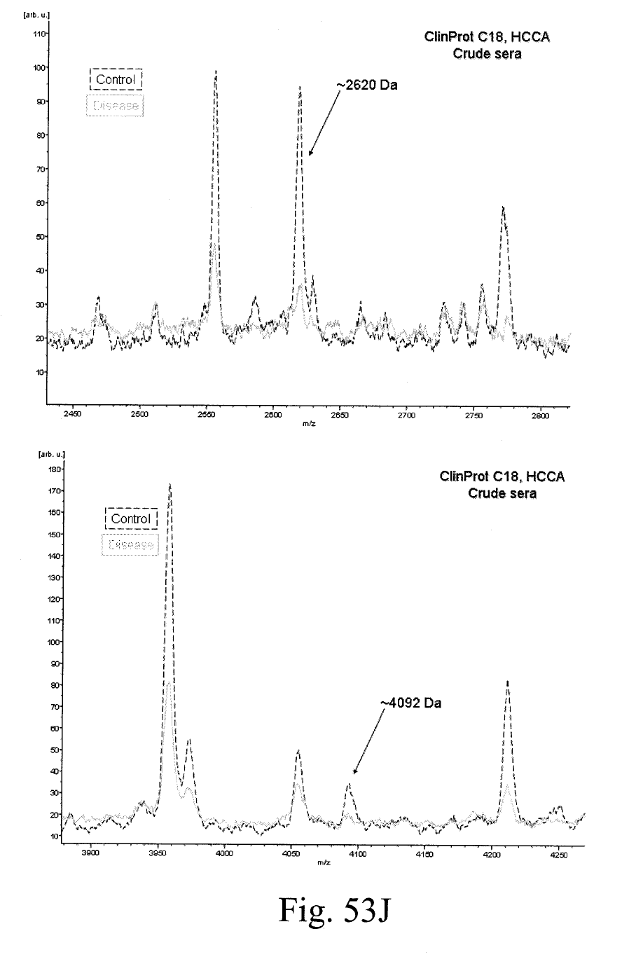

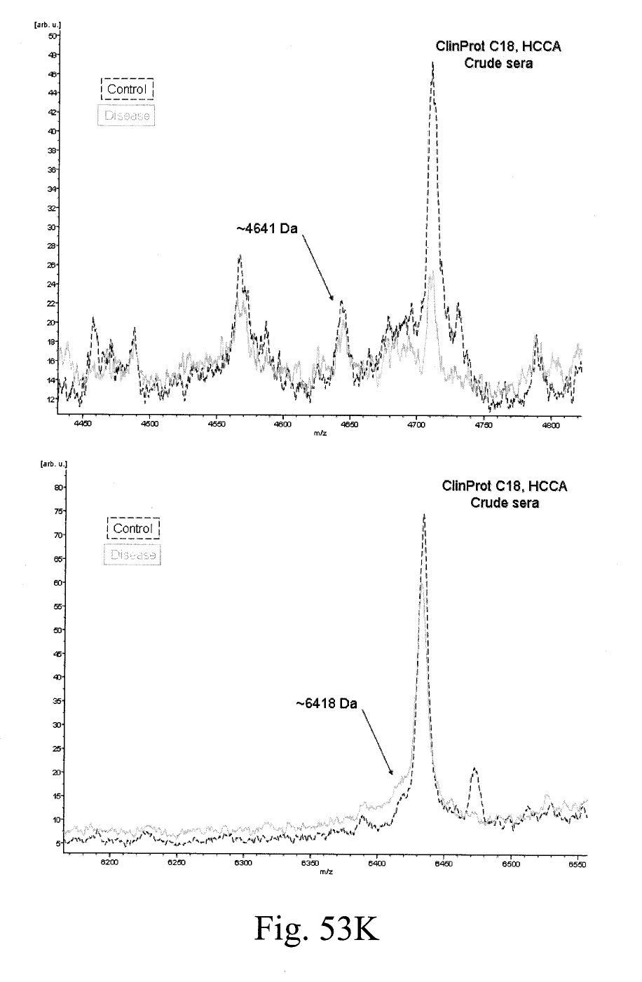

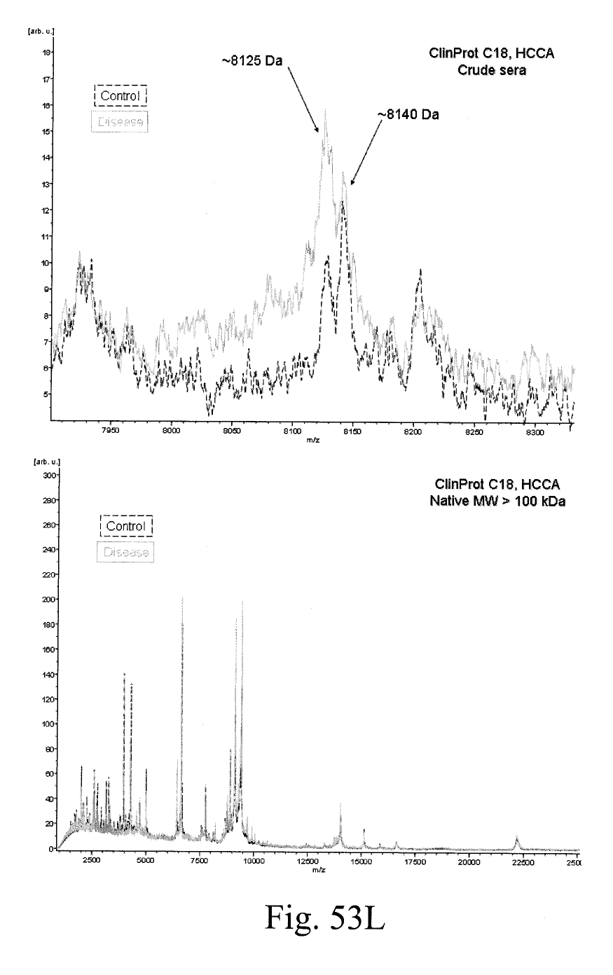

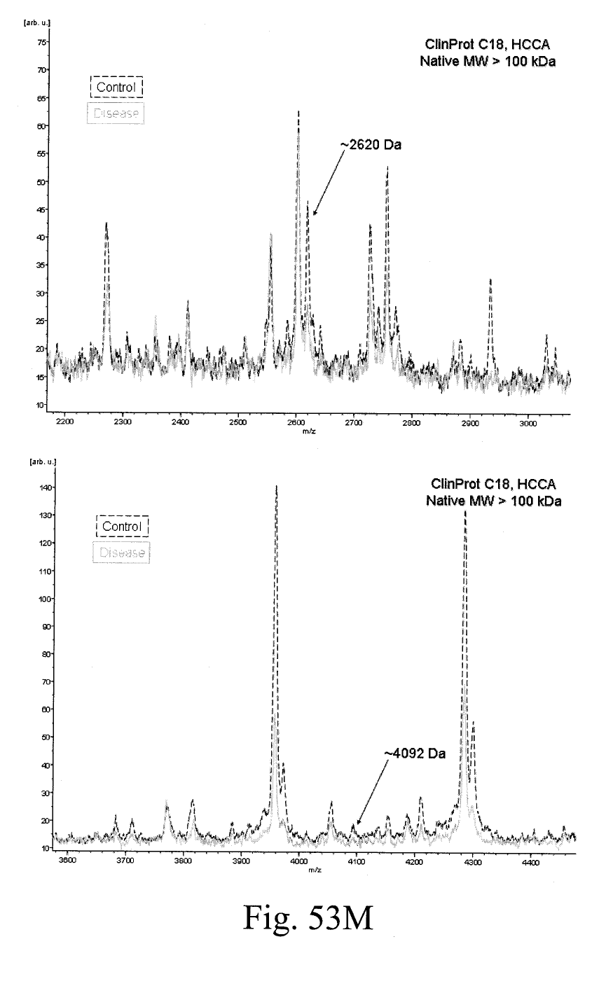

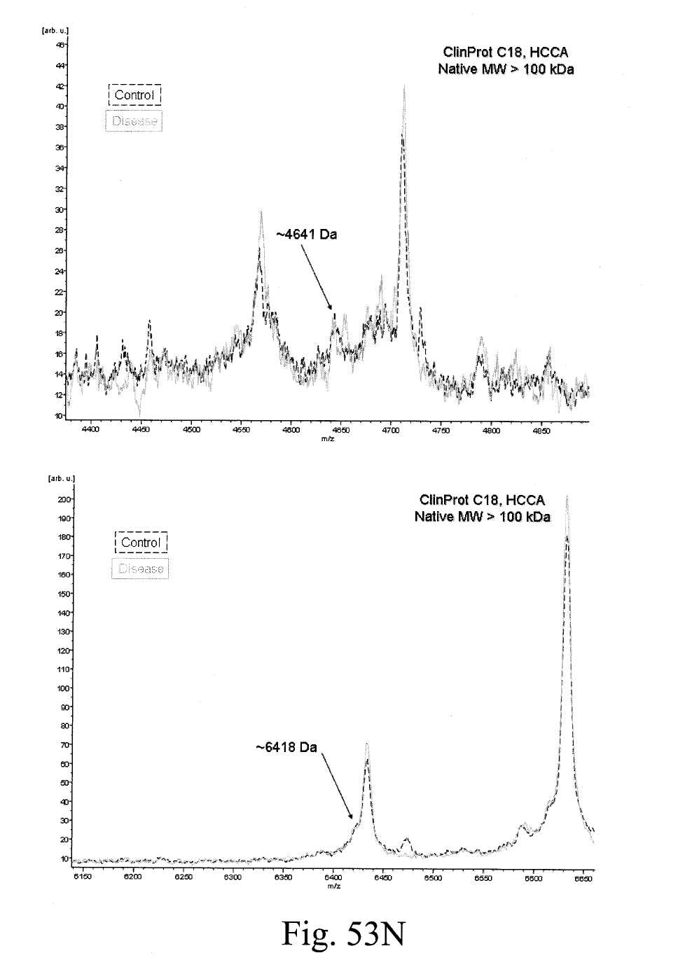

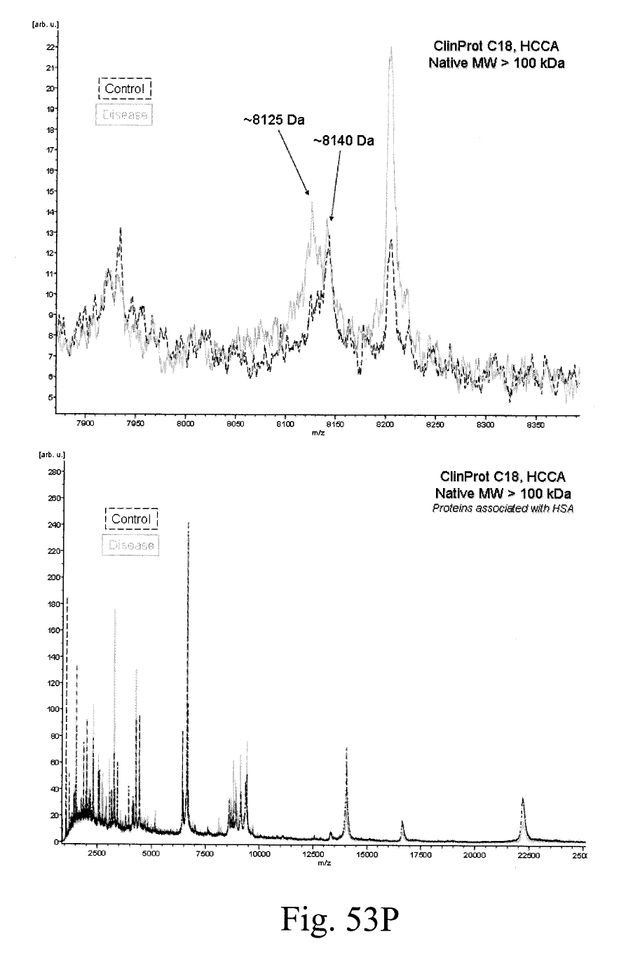

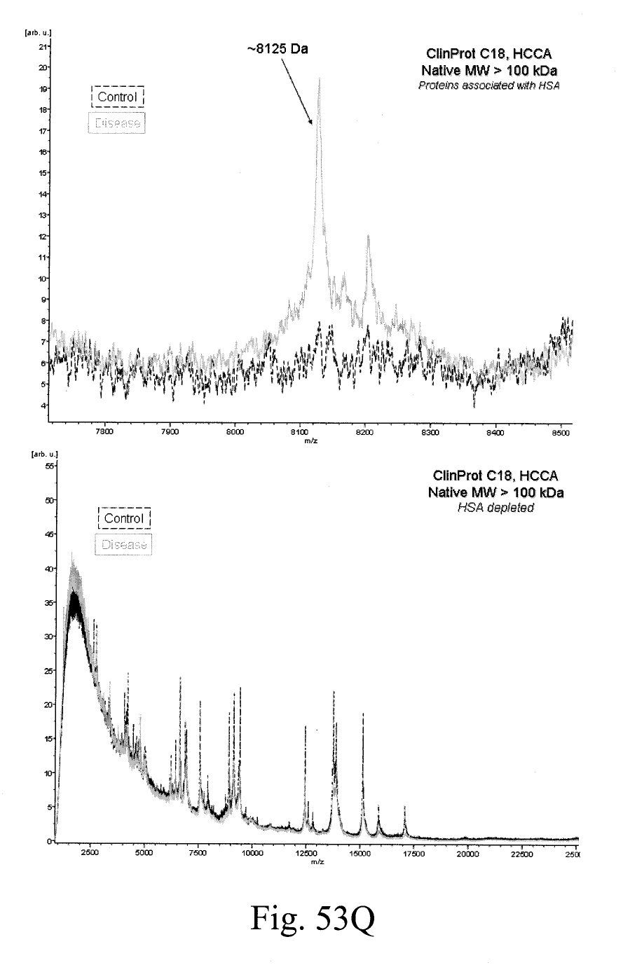

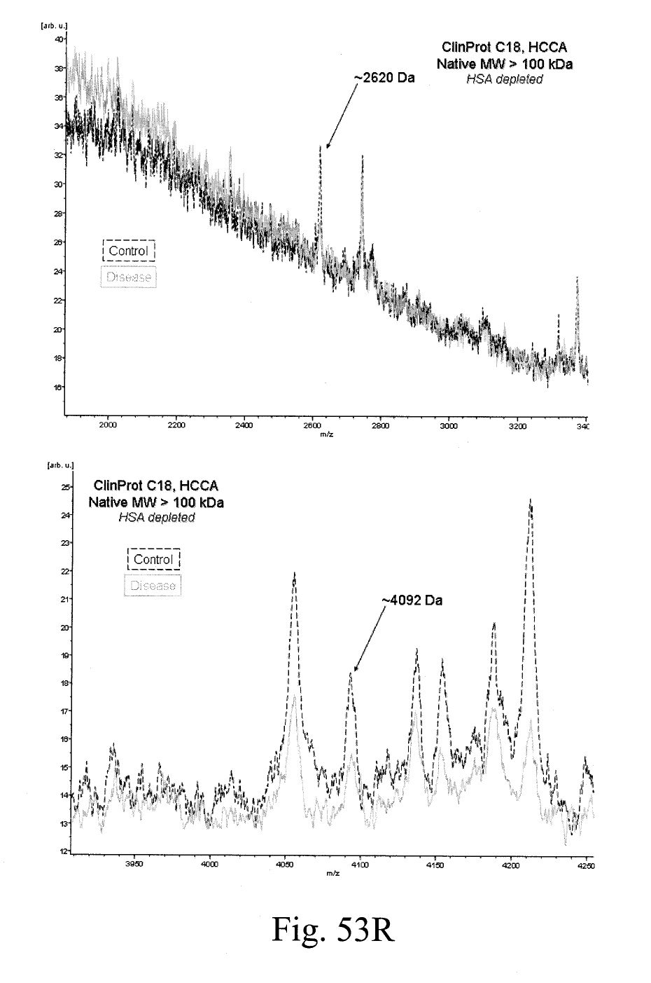

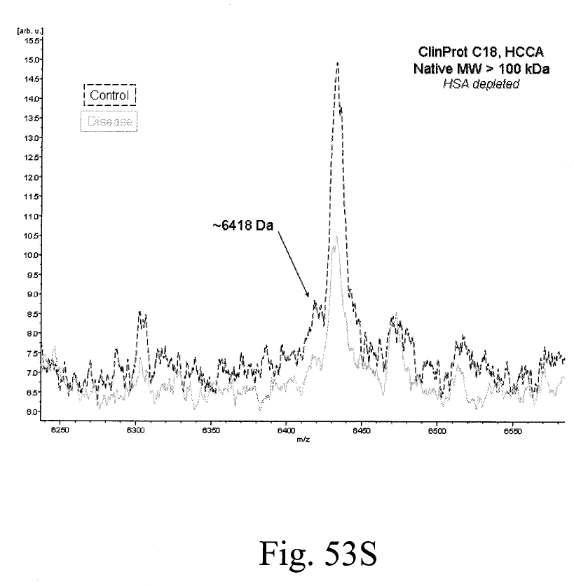

FIG. 53 presents spectra acquired with a high resolution mass spectrometer, under the conditions specified in section H-2 below. Native MW=native molecular weight.

It should be clearly understood, however, that these examples are given only by way of illustration of the subject of the invention, of which they in no way constitute a limitation.

### EXAMPLES

### A—Materials and Methods

### A-1 Biological Samples—Study Population

The biological samples of maternal blood, collected retrospectively and prospectively in a multi-center context, were taken according to the current recommendations and practices between the 14th and 25th week of pregnancy, conveyed in a centralized procedure, conserved at +4° C. according to the current procedures in force, aliquoted and frozen at −20° C. (historical samples).

With the aim of verifying the routine feasibility, the soundness of the method and the comparability of the results between various freezing conditions, and with regard to future procedures and recommendations in the field, further samples were collected on the same clinical methodological basis, taken to +4° C., but aliquoted and frozen directly at −80° C. (contemporary samples).

In all cases, the samples were stored and frozen at −80° C.

The following annotations, constituting the clinical and biological data of each sample, were recorded:

file number,

date on which the sample was taken,

mother's age and weight,

week of pregnancy adjusted for the number of days of the week when the sample was taken=number of days of gestation,

sex of the fetus,

biological markers: hCG in IU/ml, uE3 in pg/ml, α-fetoprotein in IU/ml, the 3 converted to multiples of the median (MoMs=measured value/median value),

risk, calculated according to the age of the pregnant woman and to her MoM data,

indication (age, markers, echographical clinical signs),

result of the fetal karyotype (conventional techniques),

outcome of the pregnancy (birth, medical interruption of pregnancy, FDIU fetal death in utero, optionally anatomy-pathology results).

The samples for which the outcome is known since the studies were carried out retrospectively were divided up into several groups, including one group of mothers carrying normal children and one group of mothers carrying children suffering from Down syndrome (T21). The soundness of the studies is, moreover, reinforced by the presence of mothers carrying other aberrations (Trisomie 18 or Edwards syndrome, Turner syndrome, terminal deletion of the long arm of chromosome 7 (del 7q)).

With the aim of controlling the internal validity of the studies, the samples were analyzed blind or double-blind in case-control studies (in order to be free of follow-up biases during the analyses and attrition biases).

All the retrospective studies were studies on paired samples (cases and controls matched), in order to be free of confusion biases and to make the studies comparable. A longitudinal monitoring of the experiments was carried out in order to control the experimental variability (instrument- and human-related). The size of the populations studied and the requisite power ensure the external validity of the results.

### A-2 Materials and Methods for Protein Analysis

All the research studies were carried out in an environment of good laboratory practice (GLP), in particular:

### Materials and Consumables

A mass spectrometer of PBS II c type equipped with the ProteinChip® software V.3.1.1 for spectrum acquisition and with the Bio Marker Wizard (BMW) software Version 3.1 (Ciphergen Biosystems Inc., Fremont, Calif., USA) was used.

As an addition to the studies carried out on an H50 hydrophobic surface (mixture of C6 to C12 chains) with the Ciphergen mass spectrometer, the samples were passed through a spectrometer with a higher resolution, in this case a Bruker Ultraflex spectrometer (section H-2 below and FIG. 53).

The inventors used various purification surfaces in an approach with and without fractionation, with or without albumin depletion, in reverse-phase mode, in targeted mode with antibodies, in phosphorylation, such as glycosylation, study mode, including surface chemistry and interaction chemistry, and the matrices used for the protein ionization are reiterated in table 2 below. The references indicated are, for each surface, the references of the supplier (Ciphergen Biosystems Inc., Fremont, Calif., USA).

TABLE 2

| References | Surface properties | Surface chemistry | Catalogue |
|---|---|---|---|
| C553-0028 | Hydrophobic & | C16 groups + carboxylate groups | January 2003 |
| | cationic - H4 | | |
| C553-0065 | Hydrophobic - H50 | C6 to C12 chains | September 2003 |
| C554-0065 | | | |
| C553-0043 | Normal phase - | Silicon oxide | January 2003 |
| | NP20 | | |
| C553-0045 | PS20 | Epoxy groups | January 2003 |
| C553-0027 | Anionic - SAX2/ | Quaternary amine group | January 2003 |
| C554-0052 | LSAX30 | | |
| C573-0080 | Anionic - Q10 | Quaternary amine group | September 2003 |
| C553-0026 | Cationic - WCX2/ | Carboxylate group | January 2003 |
| C554-0056 | LWCX30 | | |
| C553-0075 | Cationic - CM10 | Carboxylate group | September 2003 |
| C553-0022 | Metallic - IMAC3/ | Nitrilotriacetic acid | January 2003 |
| C554-0057 | IMAC40 | | |
| C300-0001 | Matrix - CHCA | Alpha-cyano-4-hydroxycinnamic acid | January 2003 |
| C300-0003 | Matrix - EAM | | January 2003 |
| C300-0002 | Matrix - SPA | Sinapinic acid | January 2003 |

The resolution and the precision in terms of mass were verified monthly, before, during and after each experiment, in the context of quality control procedures using tests on gold-plated chips in the MALDI mode or on silicon oxide chips in the SELDI mode and using the calibrating agents of table 3 hereinafter. The references indicated are the references of the supplier (Ciphergen Biosystems Inc., Fremont, Calif., USA).

TABLE 3

| References | Calibrating agents |
|---|---|
| C100-0004 | Equine cytochrome C, equine muscle myoglobin, |
| | rabbit GAPDH, bovine serum albumin, E. coli |
| | beta-galactosidase |
| C100-0007 | Hirudin BHVK, bovine cytochrome C, equine |
| | muscle myoglobin, bovine red blood cell |
| | carbonic anhydrase, S. cerevisiae enolase, |
| | bovine serum albumin, bovine IgGs |
| C100-0005 | Arg8-vasopressin, somatostatin, bovine insulin |
| | beta-chain, human insulin, hirudin BHVK |
| C100-0002 | Bovine insulin |
| C100-0001 | Bovine beta-lactoglobulin A, horseradish |
| | peroxidase |

The experimental conditions routinely used in a specific mode (buffers, matrices, surface chemistries, working pH, laser intensities) are always effected in a controlled environment (20° C.-25° C.); they are listed in table 4 hereinafter.

TABLE 4

| | | | Hydratation buffer (50 to | |
|---|---|---|---|---|
| Reference | Surface chemistry | Matrix | 100 μl) | pH |
| C553-0028 | C 16 + Carboxylate | SPA | acetonitrile 5% | NA |
| C553-0028 | C 16 + Carboxylate | SPA | acetonitrile 5% | NA |
| C553-0028 | C 16 + Carboxylate | SPA | acetonitrile 5% | NA |
| C553-0065/C554-0065 | C6 to C12 | CHCA | acetonitrile 50% | 7.4 physio |
| C553-0065/C554-0066 | C6 to C13 | CHCA | acetonitrile 50% | 7.4 physio |
| C553-0065/C554-0067 | C6 to C14 | CHCA | acetonitrile 50% | 7.4 physio |
| C553-0022 | Nitriloacetic acid + Cu | CHCA | CuSO₄ 100 nM | 7.4 physio |
| C553-0023 | Nitriloacetic acid + Cu | CHCA | CuSO₄ 100 nM | 7.4 physio |
| C554-0057 | Nitriloacetic acid + Hydrophobicity + Cu | CHCA | CuSO₄ 100 nM | 7.4 physio |
| C554-0057 | Nitriloacetic acid + Hydrophobicity + Cu | CHCA | CuSO₄ 100 nM | 7.4 physio |
| C554-0057 | Nitriloacetic acid + Hydrophobicity + Cu | CHCA | CuSO₄ 100 nM | 7.4 physio |
| C554-0057 | Nitriloacetic acid + Hydrophobicity + Cu | CHCA | CuSO₄ 100 nM | 7.4 physio |
| C554-0057 | Nitriloacetic acid + Hydrophobicity + Ni | CHCA | NiSO₄ 100 nM | 7.4 physio |
| C554-0057 | Nitriloacetic acid + Hydrophobicity + Ni | CHCA | NiSO₄ 100 nM | 7.4 physio |
| C554-0057 | Nitriloacetic acid + Hydrophobicity + Ni | CHCA | NiSO₄ 100 nM | 7.4 physio |
| C554-0057 | Nitriloacetic acid + Hydrophobicity + Ni | CHCA | NiSO₄ 100 nM | 7.4 physio |
| C554-0052 | Quaternary Amine Group + Latex | SPA | PBS 1X | 7.4 physio |
| C554-0052 | Quaternary Amine Group + Latex | SPA | PBS 1X | 7.4 physio |
| C554-0052 | Quaternary Amine Group + Latex | SPA | PBS 1X | 7.4 physio |
| C554-0052 | Quaternary Amine Group + Latex | SPA | Phosphate Citrate 20 nM | 4 |
| C554-0052 | Quaternary Amine Group + Latex | SPA | Phosphate Citrate 20 nM | 4 |
| C554-0052 | Quaternary Amine Group + Latex | SPA | Phosphate Citrate 20 nM | 4 |
| C554-0052 | Quaternary Amine Group + Latex | SPA | Tris Citrate 20 nM | 6 |
| C554-0052 | Quaternary Amine Group + Latex | SPA | Tris Citrate 20 nM | 6 |
| C554-0052 | Quaternary Amine Group + Latex | SPA | Tris Citrate 20 nM | 6 |
| C554-0056 | Carboxylate Group + Latex | SPA | PBS 1X | 7.4 physio |
| C554-0056 | Carboxylate Group + Latex | SPA | PBS 1X | 7.4 physio |
| C554-0056 | Carboxylate Group + Latex | SPA | PBS 1X | 7.4 physio |
| C554-0056 | Carboxylate Group + Latex | CHCA | Phosphate Citrate 20 nM | 4 |
| C554-0056 | Carboxylate Group + Latex | CHCA | Phosphate Citrate 20 nM | 4 |
| C554-0056 | Carboxylate Group + Latex | CHCA | Phosphate Citrate 20 nM | 4 |
| C554-0056 | Carboxylate Group + Latex | SPA | Phosphate Citrate 20 nM | 4 |
| C554-0056 | Carboxylate Group + Latex | SPA | Phosphate Citrate 20 nM | 4 |
| C554-0056 | Carboxylate Group + Latex | SPA | Tris Citrate 20 nM | 6 |
| C554-0056 | Carboxylate Group + Latex | SPA | Tris Citrate 20 nM | 6 |
| C554-0056 | Carboxylate Group + Latex | SPA | Tris Citrate 20 nM | 6 |
| C553-0043 | Silicon oxide | SPA | Hepes 1 mM | 7.4 |
| C553-0043 | Silicon oxide | SPA | Hepes 1 mM | 7.4 |
| C553-0043 | Silicon oxide | SPA | Hepes 1 mM | 7.4 |
| C553-0026 | Carboxylate Group | CHCA | PBS 1X | 7.4 physio |
| C553-0026 | Carboxylate Group | CHCA | PBS 1X | 7.4 physio |
| C553-0026 | Carboxylate Group | CHCA | PBS 1X | 7.4 physio |
| C553-0026 | Carboxylate Group | CHCA | Tris Citrate 20 nM | 5 |
| C553-0026 | Carboxylate Group | CHCA | Tris Citrate 20 nM | 5 |
| C553-0026 | Carboxylate Group | CHCA | Tris Citrate 20 nM | 5 |
| C553-0026 | Carboxylate Group | CHCA | Tris-HCl 50 nM | 9 |
| C553-0026 | Carboxylate Group | CHCA | Tris-HCl 50 nM | 9 |

| | | | Laser (Cf. |
|---|---|---|---|
| Reference | Incubation buffer (100 μl) | Washing buffer (100 μl) | FIG. 3) |
| C553-0028 | acetonitrile 5% | acetonitrile 5% | Int 1 |
| C553-0028 | acetonitrile 5% | acetonitrile 5% | Int 2 |
| C553-0028 | acetonitrile 5% | acetonitrile 5% | Int 3 |
| C553-0065/C554-0065 | PBS 1X + 0.1% TFA | PBS 1X + 0.1% TFA | Int 1 |
| C553-0065/C554-0066 | PBS 1X + 0.1% TFA | PBS 1X + 0.1% TFA | Int 2 |
| C553-0065/C554-0067 | PBS 1X + 0.1% TFA | PBS 1X + 0.1% TFA | Int 3 |
| C553-0022 | PBS 1X + 0.1 Triton X100 | PBS 1X | Int 1 |
| C553-0023 | PBS 1X + 0.1 Triton X100 | PBS 1X | Int 2 |
| C554-0057 | PBS 1X + 0.1 Triton X100 | PBS 1X | Int 1 |
| C554-0057 | PBS 1X + 0.1 Triton X100 | PBS 1X | Int 2 |
| C554-0057 | PBS 1X + 0.1 Triton X100 | PBS 1X | Int 3 |
| C554-0057 | PBS 1X + 0.1 Triton X100 | PBS 1X | Int 4 |
| C554-0057 | PBS 1X + 0.1 Triton X100 | PBS 1X | Int 1 |
| C554-0057 | PBS 1X + 0.1 Triton X100 | PBS 1X | Int 2 |
| C554-0057 | PBS 1X + 0.1 Triton X100 | PBS 1X | Int 3 |
| C554-0057 | PBS 1X + 0.1 Triton X100 | PBS 1X | Int 4 |
| C554-0052 | PBS 1X | PBS 1X | Int 1 |
| C554-0052 | PBS 1X | PBS 1X | Int 2 |
| C554-0052 | PBS 1X | PBS 1X | Int 3 |
| C554-0052 | Phosphate Citrate 20 nM + 0.1% Triton X100 | Phosphate Citrate 20 nM | Int 1 |
| C554-0052 | Phosphate Citrate 20 nM + 0.1% Triton X100 | Phosphate Citrate 20 nM | Int 2 |
| C554-0052 | Phosphate Citrate 20 nM + 0.1% Triton X100 | Phosphate Citrate 20 nM | Int 3 |
| C554-0052 | Tris Citrate + 0.1% Triton X100 | Tris Citrate 20 nM | Int 1 |
| C554-0052 | Tris Citrate + 0.1% Triton X100 | Tris Citrate 20 nM | Int 2 |
| C554-0052 | Tris Citrate + 0.1% Triton X100 | Tris Citrate 20 nM | Int 3 |
| C554-0056 | PBS 1X | PBS 1X | Int 1 |
| C554-0056 | PBS 1X | PBS 1X | Int 2 |
| C554-0056 | PBS 1X | PBS 1X | Int 3 |
| C554-0056 | Phosphate Citrate 20 nM + 0.1% Triton X100 | Phosphate Citrate 20 nM | Int 1 |
| C554-0056 | Phosphate Citrate 20 nM + 0.1% Triton X100 | Phosphate Citrate 20 nM | Int 2 |
| C554-0056 | Phosphate Citrate 20 nM + 0.1% Triton X100 | Phosphate Citrate 20 nM | Int 3 |
| C554-0056 | Phosphate Citrate 20 nM + 0.1% Triton X100 | Phosphate Citrate 20 nM | Int 1 |
| C554-0056 | Phosphate Citrate 20 nM + 0.1% Triton X100 | Phosphate Citrate 20 nM | Int 2 |
| C554-0056 | Tris Citrate + 0.1% Triton X100 | Tris Citrate 20 nM | Int 1 |
| C554-0056 | Tris Citrate + 0.1% Triton X100 | Tris Citrate 20 nM | Int 2 |
| C554-0056 | Tris Citrate + 0.1% Triton X100 | Tris Citrate 20 nM | Int 3 |
| C553-0043 | Hepes 1 mM | Hepes 1 mM | Int 1 |
| C553-0043 | Hepes 1 mM | Hepes 1 mM | Int 2 |
| C553-0043 | Hepes 1 mM | Hepes 1 mM | Int 3 |
| C553-0026 | PBS 1X | | Int 1 |
| C553-0026 | PBS 1X | | Int 2 |
| C553-0026 | PBS 1X | | Int 3 |
| C553-0026 | Tris Citrate 20 nM | Tris Citrate 20 nM | Int 1 |
| C553-0026 | Tris Citrate 20 nM | Tris Citrate 20 nM | Int 2 |
| C553-0026 | Tris Citrate 20 nM | Tris Citrate 20 nM | Int 3 |
| C553-0026 | Tris-HCl 50 nM | Tris-HCl 50 nM | Int 1 |
| C553-0026 | Tris-HCl 50 nM | Tris-HCl 50 nM | Int 2 |

For the targeted studies, the following mono-clonal antibodies were used:
anti-hCG and beta hCG, reference RDI-CBL75 (RDI Research Diagnostics Inc.)
anti-human alpha-fetoprotein, reference RDI-TRK4F16-4A3 (RDI Research Diagnostics Inc.),
anti-PAPP-A, reference RDI-TRK4P41-10E2 (RDI Research Diagnostics Inc.).

### Experimental Strategy Used

The experimental strategy used is shown schematically in FIG. 1.

### Preparation of Samples

### Crude or Native Sera

The samples, which have been kept frozen since they were collected, are thawed on a bed of ice for direct analysis and/or analysis after prior purification.

### Depletion

The samples are partially depleted of abundant proteins such as albumin or immunoglobulins.

### Enrichment of Transport-Molecule-Associated Proteins

The transport proteins and the molecules that are associated with them are purified, thus increasing the relative concentration of transported molecules in the fraction of the sample analyzed.

### Sera Fractionated According to Native Mass

The crude or partially depleted or enriched samples are fractionated under native conditions by separation according to the mass of the proteins.

### Summary of the Sample Preparation

The preparation of the samples is presented in schematic form in FIG. 2.

### Purification Methods

The samples were prepared according to various degrees of fractionation and purified by solid surface phase retention chromatography and/or liquid chromatography.

### Biochemical Purification

The biochemical properties specific to each protein are used in order to separate them from one another according to their apparent surface charges (ionic properties) and their hydrophobicity. In parallel, their relative concentration in the sample and/or in the analyzed fraction of the sample is studied. Four different approaches are used:

### Normal-Phase Chromatography

This chromatographic approach, which is based on the use of silicon oxide, make it possible to verify the quality of a sample and to assess the relative concentration of a protein. The association is made by means of arginines, lysines, serines and threonines.

### Anion Exchange Chromatography

A positively charged functional group (for example: quaternary amines) is used to separate the molecules that have an established anionic nature (negatively charged). Two variable parameters are generally explored: the pH at which the association-incubation phase takes place and the degree of stringency of the eluent used.

### Cation Exchange Chromatography

A negatively charged functional group (for example: carboxylate group) is used to separate the molecules that have an established cationic nature (positively charged). As for the anion exchange chromatography, various experimental combinations are applied to the association and elution phases.

### Hydrophobic Interactions

The hydrophobic interactions release the structured water surrounding the hydrophobic sectors of biomolecules. This results in increased entrophy, which makes the interactions thermodynamically favorable. Chromatography using a carbon chain (for example: C₁₈) can be used. The association and elution conditions depend on the degree of hydrophobicity of the eluent used.

### Targeted Purification

Two experimental schemes were used for a targeted protein purification approach. In a first case, the biological characteristics of the proteins to be purified reveal abilities to associate with a metal ligand and/or a chemical ligand. In the second case, the fact that the identity of the protein to be isolated is known makes it possible to use an immuno-strategy, which consequently makes use of an antibody specific for the targeted protein.

Capture by Affinity With a Ligand

This is a purification strategy based on the high affinity of a protein for a known chemical ligand. Only proteins with affinity for this ligand will be retained on the surface specifically prepared for this purpose.

Antibody-Antigen Interactions

This is a purification strategy based on knowing the targeted protein and on the existence of antibodies specific for the target. The very high affinity that exists between the antibody and the protein against which it is directed is used to separate the target from the other proteins of the sample. Simultaneous purification of factors associated with the target is possible.

### Data Acquisition Modes and Processes

### Introduction

Various data analysis modes are used. The results from the liquid chromatography purification phases can be visualized directly during the various phases of elution of the proteins by absorption spectroscopy, or indirectly, by electrophoretic mobility separation under denaturing conditions or else by mass spectrometry.

### Absorption Spectroscopy

The data are recorded at the chromatography column outlet by insertion of a UV spectrophotometer (λ=280 nm) in series before the collecting device. The absorption at this wavelength is mainly due to tryptophan and to tyrosine.

### Denaturing Electrophoresis

The various protein fractions derived from the fractionation and purification steps are denatured through the action of denaturing agents (detergents, etc.) and thermal degradation. The denatured proteins migrate in a porous phase (the gel) under electrostatic force. Their physicochemical characteristics (pI, mass, etc.) ensure good separation of the various products.

### Mass Spectrometry

The ionized proteins are separated according to their mass by means of the following technologies:

Laser Desorption Mass Spectrometry

The proteins are deposited onto an active surface (SELDI—Surface Enhanced Laser Desorption Ionization—mass spectrometry), or a passive surface (MALDI—Matrix Assisted Laser Desorption Ionization—mass spectrometry), associated with a chemical matrix that makes it possible, under vacuum, to ionize them with a laser beam. Once ionized, they are accelerated and directed under electrostatic force to a detector placed at the end of a tube under vacuum. The speed at which they reach the detector (TOF—time of flight) is proportional to the square root of their mass.

Electrospray Mass Spectrometry

The proteins are nebulized and then ionized under the action of an inert gas while passing through a highly charged very fine needle. The analyzer is most commonly a “TOF” of the same type as for the laser desorption mass spectrometry.

### Methodology for Recording and Analyzing the Spectra: Experimental Conditions for Recording, Optimization and Annotation

The recordings and the analysis of the spectra are carried out according to various protocols, which depend on the surface and on the equipment (laser intensity, sensitivity of the detector in terms of mass, detector gain, regulating of the deflector). These parameters are optimized according to the mass-range zone to be analyzed.

The normalization factors used are mainly the Total Ion Current (TIC), certain markers having a specific normalization.

FIG. 3 summarizes the various recording conditions as a function of the molecular mass of the proteins to be detected.

During the steps preliminary to the analysis, the spectra and the associated clusters are annotated according to two complementary procedures, one automatic, the other manual, coming under the art of signal processing. The two procedures are then put up against one another.

### B—Generalities Regarding the Data Management and the Analysis of Results

The results obtained for each clinical group are analyzed by direct confrontation and opposition. Various statistical analysis schemes are used: curves showing the number of true positives (sensitivity) as a function of the number of false positives (1—specificity) (ROC—Receiver Operating Characteristic), principal component analysis (PCA), analysis by classification (CART—Classification and Regression Tree), correlations, etc. The whole, tested and validated, ensures the soundness of the predictive models constructed.

### C—First Series of Studies Carried Out

### C-1 Introduction

Several studies were carried out (E1, E2, E3, E4, E5, E6), the objective of which was to optimize the visualization of the proteins in the spectral zones explained above, and to optimize their detection and the analysis of their behavior as a function of their intrinsic biochemical properties and of their interaction and association capacities. Several conditions were defined according to the chemical properties of the surfaces or of the polymers, to the incubation buffers, to the pH, and to the matrices, the function of which is to ionize the species and to facilitate their desorption, and therefore their separation under the laser pulse (see table 2 above, which describes the conditions used).

The principal characteristics of the E6 study are:
study carried out blind
retrospective/case-control study
storage of samples at the hospital: −20° C. & −80° C.
storage of samples at Neurolab: −80° C.

Three populations were studied:
control=control group
DS=Down syndrome=T21
ES=Edward syndrome=T18
a fourth population, consisting of samples frozen and stored at −80° C., reinforces the value of the peaks analyzed.

TABLE 5
Codes

| | |
|---|---|
| Control | 9, 10, 12, 13, 14, 15, 16, A9, A10, A11, A12, A13, A14, A15, |
| | A16 |
| DS | 1, 2, 3, 4, 5, 6, 7, 8, A1, A2, A3, A4, A5, A6, A7, A8 |
| ES | E3, E4, E5, E6 |
| −80° C. | A25, A26, A27, A28 |

Remarks: The annotations of the population studied and the results of the triple test (hCG, uE3, α-fetoprotein) (reference test) show that the patients A1 and 3 were not screened, the children were born suffering from Down syndrome (Trisomie 21).

The samples are pair-matched (control/DS) in order to avoid confusion biases.

Because of the excellent reproducibility of the experiments carried out in the E1 to E5 studies in triplicate, one spectrum is recorded per sample, except for controlling the intra-experiment reproducibility (for example: the samples A7 and A15 which were recorded in triplicate in the E6 study).

The properties of the study population are summarized in the following table 6 and have the following codifications:

DS=Down Syndrome=T21

ES=Edward Syndrome=T18

DM=Data missing at the report date

N/A=Not applicable (samples taken outside the period studied)

MIP=Medical Interruption of Pregnancy

LT=Lost touch. Some of the data are therefore missing.

TABLE 6
Properties and characteristics of the population studied (E6 study)

| | | Maternal | Amenorrhea | | | | | Results | | | Out- |
|---|---|---|---|---|---|---|---|---|---|---|---|
| State | Code | age | Week + Day | hCG | uE3 | AFP | Risk | TT | Karyotype | Sex | come |
| Control | 9 | 33 | 16 + 1 | 1.55 | 0.87 | 0.35 | 1/90 | False + | Normal | M | Born |
| Control | 10 | 30 | 15 + 3 | 1.58 | 0.89 | 0.99 | 1/970 | − | NA | M | Born |
| Control | 11 | 24 | 15 + 4 | 1.50 | 0.53 | 0.93 | 1/175 | False + | Normal | M | Born |
| Control | 12 | 28 | 15 + 0 | 1.22 | 0.94 | 0.80 | 1/1900 | − | NA | M | Born |
| Control | 13 | 26 | 15 + 4 | 2.37 | 0.63 | 1.03 | 1/116 | False + | Normal | F | Born |
| Control | 14 | 28 | 16 + 0 | 2.24 | 0.72 | 0.66 | 1/95 | False + | Normal | F | Born |
| Control | 15 | 22 | 17 + 1 | 1.76 | 0.74 | 0.54 | 1/182 | False + | Normal | F | Born |
| Control | 16 | 33 | 17 + 1 | 1.68 | 0.94 | 0.87 | 1/510 | − | Normal | F | Born |
| Control | A9 | 33 | 16 + 4 | 1.46 | 0.88 | 0.74 | 1/332 | − | NA | F | Born |
| Control | A10 | 33 | 16 + 3 | 1.20 | 0.63 | 0.92 | 1/474 | − | NA | LT | LT |
| Control | A11 | 24 | 16 + 3 | 1.51 | 0.59 | 0.94 | 1/254 | False + | Normal | F | LT |
| Control | A12 | 27 | 15 + 2 | 1.34 | 0.32 | 1.06 | 1/431 | − | Normal | M | Born |
| Control | A13 | 36 | 14 + 6 | 1.97 | 0.45 | 0.86 | 1/11 | False + | Normal | F | Born |
| Control | A14 | 34 | 15 + 2 | 1.36 | 0.6 | 0.7 | 1/76 | False + | Normal | M | Born |
| Control | A15 | 38 | 17 + 5 | 1.80 | 0.94 | 1.37 | 1/239 | False + | Normal | F | Born |
| Control | A16 | 38 | 15 + 2 | 1.87 | 0.93 | 1.53 | 1/166 | False + | Normal | M | Born |
| Patient | 1 | 35 | 16 + 1 | 4.02 | 0.70 | 0.42 | 1/10 | + | DS | M | MIP |
| Patient | 2 | 27 | 15 + 5 | 4.02 | 0.42 | 1.07 | 1/8 | + | DS | M | MIP |
| Patient | 3 | 25 | 15 + 3 | 2.68 | 1.00 | 0.70 | 1/560 | False − | DS | M | Born |
| Patient | 4 | 32 | 15 + 0 | 2.22 | 1.26 | 0.77 | 1/660 | False − | DS | M | MIP |
| Patient | 5 | 31 | 15 + 4 | 2.22 | 0.62 | 1.32 | 1/126 | + | DS | F | MIP |
| Patient | 6 | 29 | 15 + 6 | 2.41 | 0.62 | 1.06 | 1/84 | + | DS | F | MIP |
| Patient | 7 | 36 | 17 + 1 | 3.99 | 0.46 | 1.12 | 1/5 | + | DS | F | MIP |
| Patient | 8 | 21 | 16 + 4 | 2.57 | 0.60 | 1.28 | 1/143 | + | DS | F | MIP |
| Patient | A1 | 28 | 16 + 3 | 1.61 | 0.82 | 0.71 | 1/441 | False − | DS | F | Born |
| Patient | A2 | 36 | 16 + 4 | 1.11 | 0.66 | 0.69 | 1/153 | + | DS | M | Born |
| Patient | A3 | 30 | 15 + 4 | 3.46 | 0.52 | 1.33 | 1/17 | + | DS | F | MIP |
| Patient | A4 | 31 | 16 + 4 | 1.49 | 0.31 | 0.56 | 1/37 | + | DS | M | MIP |
| Patient | A5 | 34 | 16 + 3 | 3.87 | 0.63 | 0.48 | 1/5 | + | DS | F | MIP |
| Patient | A6 | 30 | 16 | 2.39 | 0.55 | 0.36 | 1/10 | + | DS | M | MIP |
| Patient | A7 | 38 | 17 + 2 | 1.29 | 1.26 | 0.61 | 1/310 | False − | DS | F | MIP |
| Patient | A8 | 40 | 17 | 1.84 | 0.76 | 0.81 | 1/32 | + | DS | M | MIP |
| Patient | E3 | 30 | 16 | 0.97 | 0.43 | 0.58 | 1/57 | + | ES | M | MIP |
| Patient | E4 | 35 | 16 + 1 | 0.54 | 0.21 | 1.33 | 1/9400 | False − | ES | F | MIP |
| Patient | E5 | 37 | 16 + 5 | 0.14 | 0.44 | DM | 1/2700 | False − | ES | M | MIP |
| Patient | E6 | 37 | 15 + 2 | 0.42 | 0.64 | 0.54 | 1/590 | False − | ES | M | MIP |
| Patient | A25 | 35 | 17 + 5 | 3.79 | 0.69 | 0.64 | 1/6 | + | DS | M | MIP |
| Patient | A26 | 34 | 15 + 1 | 4.91 | 0.99 | 0.83 | 1/22 | + | DS | F | MIP |
| Patient | A27 | 35 | 23 + 4 | 2.64 | 0.98 | 1.22 | 1/184 | N/A | DS | M | MIP |
| Patient | A28 | 34 | 20 | 3.95 | 1.50 | 1.61 | 1/920 | N/A | DS | F | MIP |

In this specific application (selected conditions documented), the samples were either analyzed native unfractionated, or analyzed fractionated as a function of the mass of the proteins. The eluates were used either native or analyzed after depletion of albumin or other transport proteins, and then separated and purified according to two modes, one anionic, the other hydrophobic. An analyzer of SELDI-TOF mass spectrometer type was used to acquire the spectral information.

### Interest, Associativity and Purification on an Anionic Surface

The spectra presented are obtained on a separative surface of anionic type. The peaks of interest mentioned are present at pH 4 and at any pH value above 4, insofar as no phenomenon of aggregation or precipitation of the proteins occurs at basic pH.

### Interest, Associativity on a Hydrophobic Surface

The spectra presented are obtained on a separative surface of hydrophobic type, after a phase of incubation and of washing in phosphate buffered saline (1× PBS) at pH 7.4 (Sambrook, J., Fritsch, E. F. & Maniatis, T. (1989). Molecular Cloning: A laboratory manual, Cold Spring Harbor Laboratory Press, Cold Spring Harbor, N.Y.).

### C-2 Experimental Scheme (E1, E2, E3, E4 and E6 Studies)

FIG. 4 presents the experimental scheme that was used.

During profiling studies, the inventors worked under nondenaturing conditions as under denaturing conditions. Protein chips were prepared according to the following procedure:

1. Insertion of the arrays into the bioprocessors; 2. Hydration or prerinsing of the spots with 5 to 100 μl of a specific hydration buffer; 3. Removal of the supernatant and addition of 100 μl of an incubation buffer; 4. Addition of 1 μl of serum per spot; 5. Incubation for 10 minutes to 1 h 45; 6. Removal of the supernatant; 7. Washing of the surface with 100 μl of a specific washing buffer; 8. Removal of the supernatant; 9. Addition of the matrix (CHCA or SPA); 10. Drying.

The protein chips are inserted into the mass spectrometer or any other suitable reading or sequencing device.

The various separating and purifying conditions used and their preparation protocols are reiterated in table 2 above.

In another, targeted approach, study (of phosphorylation type or using an antibody) (E2 study), the inventors prepared protein chips with unfractionated native or fractionated biological samples, according to the following procedure:

1. Insertion of the arrays into the bioprocessors; 2. Hydration or prerinsing of the spots with 20 μl of a specific hydration buffer, twice 5 minutes; 3. Association of the capture molecules; 4. Elimination of the supernatant and addition of 100 μl of an incubation buffer, 3 times 5 minutes; 5. Addition of 1 μl of serum to each spot; 6. Incubation for 30 minutes; 7. Removal of the supernatant; 8. Washing of the surface with 3 times 100 μl, 5 minutes, of a specific washing buffer; 9. Removal of the supernatant and final washing with 100 μl of pure water; 10. Addition of the matrix (CHCA or SPA diluted to 1/10); 11. Drying.

The protein chips are inserted into the mass spectrometer for reading according to the various intensities described above and in FIG. 3, or into any other suitable reading device, optimized and regulated accordingly. Those skilled in the art will choose the intensity of the laser so as to make it possible to cause flight of the proteins, as a function of their mass, of some of their physicochemical properties and of any interactions that they establish with partners. The antibodies used were described above.

The calibrating agents used on a normal phase/silicon oxide chip and in the Maldi mode correspond to those described in table 3.

In these various studies, separation with fractionation was carried out, generating the following four fractions:
Fraction A: molecular weight≦10 kDa
Fraction B: 10 kDa<molecular weight≦50 kDa
Fraction C: 50 kDa<molecular weight≦100 kDa
Fraction D: molecular weight>100 kDa

The aim of the fractionation according to mass is to increase the relative concentration of proteins of one fraction relative to the concentration of total proteins, without however modifying the absolute concentration of the proteins separated. In fraction C, albumin is normally found to be the predominant element, and can, where appropriate, be specifically eliminated. This elimination was not carried out in the experiments described here; the elimination of albumin will make it possible to detect an additional peak, corresponding to α-fetoprotein, which is for the moment masked by the albumin peak.

These fractions were obtained using separating devices of the Vivaspin 500 type from the company Vivascience, with 3 separate cutoff thresholds: 10, 50 and 100 kDa. The membrane-filtration separation was carried out in 1×PBS, pH 7.4, under nondenaturing conditions. Everything that passes through the membrane has a lower mass than the cutoff threshold, everything that remains in the dead volume above the membrane has a mass higher than the cutoff threshold. The proteins remaining in the upper part of the Vivaspin 500 with a cutoff threshold of 100 kDa therefore have a native mass >100 kDa. This also extends to both heterogeneous and homogeneous multimeric complexes.

### C-3 Instrumentation (Example: E6 Study—Anionic Purification)

### Acquisition Parameters

### Acquisition System: Proteinchip Biomarker System Type II.c

TABLE 7
General acquisition parameters of the principal study (E6)

| | Acquisition parameters |
|---|---|
| Recording protocol | Instrument: |
| 1. Set the high mass to 50 000 | Type: PBS IIc |
| daltons, optimized from 2500 daltons | Serial Number: 2F359TC |
| to 15 000 daltons. | Acquisition: |
| 2. Set the starting laser intensity to 170. | Digitizer frequency: 500.0 MHz |
| 3. Set the detector sensitivity to 10. | Ionic mode: Positive |
| 4. Set the deflector mass to 2500 | Source voltage: 20 000 V |
| daltons. | Detector voltage: 2900 V |
| Recording statistics | Pulse voltage: 3000 V |
| Shots: | Pulse duration: 700 ns |
| Carried out: 2814; Conserved: 2680; | Mass focus: 8750.00 Da |
| High: 0; Low: 0 | Deflector mode: Manual |
| Intensity: | Deflector mass: 2500 Da |
| Low: 170; High: 175 | |
| Sensitivity: | |
| Low: 10; High: 10 | |
| Recording period | |
| E6 study | |

TABLE 8
Specific acquisition parameters of the principal study (E6)

| Samples | Vacuum | Samples | Vacuum |
|---|---|---|---|
| A1 | 1.935e−007 Torr | 1 | 2.072e−007 Torr |
| A2 | 1.673e−007 Torr | 2 | 1.798e−007 Torr |
| A3 | 1.575e−007 Torr | 3 | 1.673e−007 Torr |
| A4 | 1.575e−007 Torr | 4 | 1.575e−007 Torr |
| A5 | 2.072e−007 Torr | 5 | 1.673e−007 Torr |
| A6 | 1.798e−007 Torr | 6 | 1.575e−007 Torr |
| A7 (1) | 1.673e−007 Torr | 7 | 1.477e−007 Torr |
| A7 (2) | 1.380e−007 Torr | 8 | 1.380e−007 Torr |
| A7 (3) | 1.380e−007 Torr | 9 | 1.935e−007 Torr |
| A8 | 1.575e−007 Torr | 10 | 1.798e−007 Torr |
| A9 | 1.798e−007 Torr | 11 | 1.673e−007 Torr |
| A10 | 1.673e−007 Torr | 12 | 1.575e−007 Torr |
| A11 | 1.575e−007 Torr | 13 | 1.575e−007 Torr |
| A12 | 1.477e−007 Torr | 14 | 1.477e−007 Torr |
| A13 | 1.935e−007 Torr | 15 | 1.380e−007 Torr |
| A14 | 1.935e−007 Torr | 16 | 1.380e−007 Torr |
| A15 (1) | 1.380e−007 Torr | E3 | 1.282e−007 Torr |
| A15 (2) | 1.673e−007 Torr | E4 | 1.282e−007 Torr |
| A15 (3) | 1.673e−007 Torr | E5 | 1.575e−007 Torr |
| A16 | 1.282e−007 Torr | E6 | 1.477e−007 Torr |

### Spectrum Normalization Report

The “Total Ion Current” (TIC) normalization method makes it possible, for each spectrum, to produce the mean of the intensities, and adjust the mean intensities of each spectrum such that the data are normalized.

The normalization parameters used corresponded to a “Total Ion Current” normalization beginning at 3000 and ending at 20 000, with subtraction of the base line, a normalization coefficient of 0.269112, and with no normalization relative to the mass.

TABLE 9
Normalization report (Principal study-Anionic separation)

| Sample | Normalization | Used in | | Sample | Normalization | Used in | |
|---|---|---|---|---|---|---|---|
| name | factor | normalization? | With TIC | name | factor | normalization? | With TIC |
| A01 | 1.255852 | yes | 0.214286 | 1 | 2.450139 | yes | 0.109835 |
| A02 | 1.664225 | yes | 0.161704 | 2 | 0.808214 | yes | 0.332971 |
| A03 | 2.185387 | yes | 0.123142 | 3 | 1.07021 | yes | 0.251457 |
| A04 | 1.291102 | yes | 0.208436 | 4 | 2.306647 | yes | 0.116668 |
| A05 | 0.974679 | yes | 0.276103 | 5 | 0.743689 | yes | 0.361861 |
| A06 | 0.648508 | yes | 0.414971 | 6 | 0.974983 | yes | 0.276017 |
| A07 | 0.560603 | yes | 0.48004 | 7 | 2.918434 | yes | 0.092211 |
| A07 | 1.307443 | yes | 0.205831 | 8 | 0.719411 | yes | 0.374073 |
| A07 | 1.895244 | yes | 0.141993 | 9 | 0.975285 | yes | 0.275932 |
| A08 | 0.821502 | yes | 0.327585 | 10 | 1.17547 | yes | 0.22894 |
| A09 | 1.261377 | yes | 0.213348 | 11 | 2.192259 | yes | 0.122756 |
| A10 | 0.751794 | yes | 0.35796 | 12 | 1.508624 | yes | 0.178382 |
| A11 | 1.233307 | yes | 0.218204 | 13 | 1.387028 | yes | 0.194021 |
| A12 | 1.249535 | yes | 0.21537 | 14 | 1.690433 | yes | 0.159197 |
| A13 | 1.068304 | yes | 0.251906 | 15 | 1.568808 | yes | 0.171539 |
| A14 | 0.600652 | yes | 0.448034 | 16 | 1.648534 | yes | 0.163243 |
| A15 | 1.233057 | yes | 0.218248 | E3 | 1.89569 | yes | 0.14196 |
| A15 | 1.263684 | yes | 0.212958 | E4 | 1.437991 | yes | 0.187144 |
| A15 | 3.701159 | yes | 0.07271 | E5 | 1.241774 | yes | 0.216716 |
| A16 | 1.244716 | yes | 0.216204 | E6 | 2.593202 | yes | 0.103776 |

### Calibration Report

The calibration is carried out as a function of the mass range observed. In the zone 3 to 20 kDa, three calibrations are used, depending on whether the calibrating agent is:

bovine insulin,

bovine cytochrome C,

bovine β-lactoglobulin A.

For example, between 2.9 and 5.8 kDa, the calibration is performed on the peaks for insulin (bovine) (5733.58+1H) and for bovine insulin 2H+(2866.79+1H).

### Calibration Equation

The calibration equation used is quadratic:

$$\frac{m / z}{U} = {a ( t - {t}_{0} )}^{2} + b$$

where m/z is the mass-to-charge ratio, U the source voltage and t the time of flight.

### Acquisition Parameters (Example: the Case of Insulin)

Two spectrum recording conditions, for two concentrations of bovine insulin, were used.

TABLE 10
Recording conditions

| | Acquisition parameters |
|---|---|
| Recording protocol: | Instrument: |
| Protocol 1, Bovine INSULIN | Type: PBS IIc |
| 1. Set the high mass to 50 000 daltons, | Serial Number: 2F359TC |
| optimized from 1000 daltons to 10 000 | Acquisition: |
| daltons. | Digitizer frequency: 500.0 MHz |
| 2. Set the starting laser intensity to 120. | Ion mode: Positive |
| 3. Set the detector sensitivity to 10. | Source voltage: 20 000 V |
| 4. Set the deflector mass to “auto”. | Detector voltage: 2900 V |
| Recording statistics | Pulse voltage: 3000 V |
| Shots: | Pulse duration: 564 ns |
| Carried out: 682; Conserved: 620; | Mass focus: 5500.00 Da |
| High: 0; Low: 0 | Deflector mode: Auto |
| Intensity: | Deflector mass: 1000 Da |
| Low: 120; High: 130 | |
| Sensitivity: | |
| Low: 10; High: 10 | |
| Recording period | |
| E6 study | |

### Results of the Acquisition After Calibration (the Case of Insulin)

TABLE 11

| Parameters | Values | Parameters | Values | |
|---|---|---|---|---|
| a | 0.358981048675136 × 10⁻³ | t₀ | 1.11897791939812 | $\frac{m / z}{U} = {a ( t - {t}_{0} )}^{2} + b$ |
| b | 8.2752735372077 × 10⁻³ | U | 20 000 V | |

After calibration, the difference with respect to theory is equal to 0.00 dalton (i.e. accuracy greater than two orders of magnitude).

### Clusterization Report

### Definition

In order to compare the level of expression of a protein according to the samples, clusterization consists in grouping together the value of the intensity of the peaks corresponding to this protein for all the spectra recorded. A cluster is therefore defined as a table yielding the value of the intensity of the peak considered for each spectrum studied.

### Clusterization

Three groups are simultaneously clusterized so as to be able to make a subsequent comparative:

Controls (Control), DS (Down Syndrome) and ES (Edward Syndrome).

The annotations were made according to two complementary modes, one manual, by the experimenters, the other automatic.

### Manual Clusterization Mode

The peaks are manually annotated.

The clusters are then completed by means of a search in a window of ±0.3% of the value M+H for a signal such as signal/noise=2, otherwise the local background noise is used.

### Automatic Clusterization Mode

The detection of the peaks obeys 2 criteria:
the signal-to-noise ratio must be ≧10
the peak must be present in 30% of the spectra.

The clusters are then completed by means of a search in a window of ±0.3% of the value M+H for a signal such as signal/noise=2, otherwise the local background noise is used.

### Conclusion With Regard to the Spectral Zone Visualized in the Example Chosen

Given the spectral quality (background noise, quality of the signal and of the signal/noise ratio):

the visualization zone is between 3000 Da and 20 000 Da,

the analysis zone is between 4000 Da and 20 000 Da.

The calibration is carried out on the peaks for insulin (bovine) (5733.58+1H) and for bovine insulin 2H+(2866.79+1H). The objective is to optimize the calibration zone between 2800 and 5750 Da. Between 6 and 12 kDa, cytochrome C (bovine) is used for the calibration. Between 9 and 19 kDa, β-lactoglobulin A (bovine) is used for the calibration.

The choice of the calibrating agent does not in any way influence the intensity values. Their values do not vary as a function of the calibration used. Calibration makes it possible to increase the accuracy of the peak mass values recorded, on a restricted spectral zone.

In addition to the calibrating agents mentioned above, other calibrating agents may be used. Table 12 hereinafter gives a list of calibrating agents that can be used, and also their molecular weights.

TABLE 12

| | Molecular weight |
|---|---|
| Peptide/protein | (Daltons) |
| Arg8-Vasopressin | 1084.2474 |
| Human angiotensin I | 1296.5 |
| [Glu¹] Fibrinopeptide B | 1570.6 |
| Somatostatin | 1637.9030 |
| Porcine Dynorphine A (209-225) | 2147.5 |
| Human ACTH (1-24) | 2933.5 |
| Human β-endorphin | 3465.0 |
| Bovine insulin β-chain | 3495.9409 |
| Bovine insulin | 5733.6 |
| Human insulin | 5807.6533 |
| Bovine ubiquitin | 8564.8 |
| Bovine cytochrome C | 12230.9 |
| Equine cytochrome C | 12360.2 |
| Bovine superoxide dismutase | 15591.4 |
| Equine myoglobin | 16951.1 or 16951.5* |
| Bovine β-lactoglobulin A | 18363.3 |
| Rabbit GAPDH | 35688.0 |
| Horseradish peroxidase | 43240.0 |
| Bovine serum albumin (BSA) | 66410.0 or 66433.0* |
| Hirudin BHVK | 7033.6136 |
| Chicken conalbumin | 77490.0 |
| E. coli β-galactosidase | 116351.0 |
| Bovine IgG | 147300.0 |

*indication of molecular weight different according to suppliers.

### D—Results of the First Series of Studies

### D-1 Summary of the Analytical Results Obtained

Analytical Results=Spectral resolution in terms of mass and in terms of intensity

The inventors identified clusters of interest (references in tables 13 and 14) in the data derived from the spectra obtained (one spectrum per sample of the populations of the three groups Control, DS (T21) and ES (T18), apart from the −80° C. group, except in the specific case of samples A7 and A15, recorded in triplicate in the E6 study). The number of manually annotated clusters is reduced according to the criterion “present in at least 30% of the spectral population”.

TABLE 13
Analytical phase summary

| | | | | Number | | |
|---|---|---|---|---|---|---|
| | | | | of | Number of | Number |
| | | Analysis | Clusterization | clusters | clusters | of spectra |
| Experiments | | zone | mode | identified | selected | analyzed |
| Anionic | E6 | 4-20 kDa | Manual | 156 | 29 (18.6%) | 40 |
| separating | | | Automatic | 8 | 7 (87.5%) | |
| phase | E1-E5 | 3-10 kDa | Manual | 40 | 39 (97.5%) | 32 |
| | | | Automatic | 6 | 6 (100%) | |
| Hydrophobic | E6 | 3-10 kDa | Manual | 140 | 31 (22.2%) | 34 |
| interaction | | | Automatic | 17 | 17 (100%) | |
| | | 3-20 kDa | Manual | 164 | 54 (32.9%) | 34 |
| | | | Automatic | 35 | 34 (97.2%) | |

TABLE 14
List of clusters selected

| | Anionic separating | | | Presence of |
|---|---|---|---|---|
| | phase | | Hydrophobic | a signal on |
| | | Accumulation | interaction | samples frozen |
| CLUSTERS | E6 | E1-E5 | E6 | at −80° C. |
| MW3061 | | | +* | − |
| MW3101 | | | + | + |
| MW3156 | | | + | + |
| MW3166 | | | +* | + |
| MW3181 | | | +* | + |
| MW3214 | | | + | + |
| MW3223 | | | + | + |
| MW3233 | | | +* | + |
| MW3313 | | | + | + |
| MW3320 | + | − | | + |
| MW3321 | | | + | − |
| MW3371# | + | + | | + |
| MW3394 | | | +* | − |
| MW3400 | + | − | | + |
| MW3442# | + | + | | + |
| MW3486# | + | + | | + |
| MW3504 | | | +* | + |
| MW3678 | | | + | + |
| MW3705 | | | + | + |
| MW3815 | + | − | | ++ |
| MW3846 | + | − | | ++ |
| MW4059 | | | + | + |
| MW4067 | | | + | + |
| MW4110 | + | − | | + |
| MW4111 | | | + | + |
| MW4152# | + | + | | + |
| MW4168# | + | + | | + |
| MW4186 | + | + | | + |
| MW4197 | | | + | − |
| MW4201# | + | + | | + |
| MW4217# | − | + | | + |
| MW4279 | | | +* | + |
| MW4286 | | | + | + |
| MW4295 | | | + | + |
| MW4300# | + | + | | + |
| MW4317 | | | + | + |
| MW4332 | + | + | | + |
| MW4349 | | | + | + |
| MW4410 | | | + | + |
| MW4412 | − | + | | + |
| MW4463 | | | + | + |
| MW4470 | | | + | + |
| MW4530 | − | + | | + |
| MW4583 | | | +* | + |
| MW4650 | | | + | + |
| MW4682# | | | +* | − |
| MW4706 | | | +* | + |
| MW4722 | | | +* | + |
| MW4816 | | | + | + |
| MW4821 | + | + | | + |
| MW4860 | − | + | | + |
| MW4893 | − | + | | + |
| MW4935 | − | + | | + |
| MW5008 | − | + | | + |
| MW5033 | | | + | + |
| MW5222 | − | + | | + |
| MW5625# | | | + | + |
| MW5630 | − | + | | + |
| MW5695# | + | + | | + |
| MW5712 | − | + | | + |
| MW5720 | | | +* | − |
| MW5756 | | | + | + |
| MW5793 | − | + | | + |
| MW5799 | | | + | + |
| MW5982 | − | + | | + |
| MW6222# | + | + | | + |
| MW6299 | + | + | | + |
| MW6423# | | | + | + |
| MW6430 | − | + | | + |
| MW6439# | | | + | + |
| MW6461 | | | + | + |
| MW6470 | − | + | | + |
| MW6482 | | | + | + |
| MW6548 | − | + | | + |
| MW6605 | | | +* | + |
| MW6620# | | | | + |
| MW6624 | − | + | | + |
| MW6636# | | | + | + |
| MW6659 | | | + | + |
| MW6679 | | | + | + |
| MW6764 | | | +* | + |
| MW6782 | | | + | − |
| MW6808 | − | + | | + |
| MW6937 | + | + | | + |
| MW7019 | − | + | | + |
| MW7020 | | | + | + |
| MW7652 | − | + | | + |
| MW8112 | | | + | + |
| MW8176 | + | + | | + |
| MW8206 | + | + | | + |
| MW8220 | | | + | − |
| MW8226 | − | + | | + |
| MW8558 | − | + | | + |
| MW8563 | | | + | + |
| MW8581 | | | +* | − |
| MW8594 | + | + | | + |
| MW8627 | | | + | + |
| MW8691 | | | + | + |
| MW8695 | | | | + |
| MW8717 | | | +* | + |
| MW8809 | | | + | + |
| MW8872 | | | +* | + |
| MW8917 | | | +* | + |
| MW8929 | | | + | + |
| MW9291 | | | + | + |
| MW9358 | + | + | | + |
| MW9360# | | | + | + |
| MW9401 | | | +* | − |
| MW9407 | | | +* | + |
| MW9617 | | | +* | − |
| MW10051 | | | + | − |
| MW11093 | | | +* | − |
| MW11369 | + | N/A | | + |
| MW12434 | + | N/A | | + |
| MW12575 | + | N/A | | + |
| MW12862 | | | + | − |
| MW13313 | + | N/A | | + |
| MW13316 | | | + | + |
| MW14027 | + | N/A | | + |
| MW14035# | | | + | + |
| MW14430 | | | + | − |
| MW16672 | + | N/A | | + |
| MW16995.56 | + | N/A | | |
| MW17262# | | | + | + |
| MW17348.75 | + | N/A | | + |
| MW17386 | | | + | + |
| MW17581.18 | + | N/A | | + |
| MW18901.94 | + | N/A | | + |
| MW19139.2 | + | N/A | | + |
| MW19558.27 | + | N/A | | + |
| MW19624.73 | + | N/A | | + |
| MW19845.94 | + | N/A | | + |

Legend of table 14:
+*: Less stringent cluster selection criterion applied to the separating phase with hydrophobic interactions: → [% present control group] + [% present T21 group] >=30
++: Cluster exhibiting a signal on samples stored at −80° C., 10 times greater than with samples stored at −20° C.
#Clusters subsequently presented by way of example.
−: No observation.

The clusters defined in table 14 correspond to the markers of list (i) above.

### D-2 Quality of the Data and Observations

Several qualitative criteria make it possible to judge the quality of the data. By way of examples, mention may be made of the reproducibility of the spectra obtained for the same sample, the differences in time of flight (proportional to the mass of the proteins analyzed) observed for the same cluster, the parameters for normalization of the spectra, or alternatively the value of the signal-to-noise ratio, the coefficients of variation often much less than 10%, etc.

Table 15 shows an example of good reproducibility of the TOF value (therefore of the real mass observed) and of the quality of the signal-to-noise ratio on the E1 to E6 studies.

Table 16, also presented by way of example, indicates, for some clusters observed in the experiments of the E6 study, the time of flight (TOF), the value p of the cluster and, for each group (control, T21, T18), the mean, minimum and maximum intensities and also the standard deviation with respect to the intensity.

TABLE 15
Experiment Q10 accumulation E1 to E5/automatic annotation mode
PARTIAL TABLE PRESENTED BY WAY OF EXAMPLE

| Definition of the cluster | | | | | | | | | | |
|---|---|---|---|---|---|---|---|---|---|---|
| | Mean mass | | | | | | | | | |
| | of the | | | | | | | | | |
| | substances | | | | Validity of the cluster | | | | | |
| | (calibration | | | | Representation | | | | | |
| | according | | | | of the cluster | | | | | |
| | to bovine | | | | | % peaks detected | Time of flight | | | |
| | cytochrome | Annotation | Cluster | Calibrating | % | during the | TOF | Deviation | Minimum | Maximum |
| Cluster | C) | mode | number | agent | manual annotations | clusterization | (μs) | (ns) | (μs) | (μs) |
| MW4152 | 4151.00/ | Automatic | 1 | Bovine | N/A | 100.00 | 24.67 | 18.93 | 24.65 | 24.69 |
| | 4163.88 | | | insulin | | | | | | |
| MW4168 | 4166.38/ | | 2 | (50% of the | N/A | 72.00 | 24.71 | 16.91 | 24.69 | 24.74 |
| | 4179.04 | | | spectral | | | | | | |
| MW4186 | 4184.41/ | | 3 | population)/ | N/A | 66.67 | 24.76 | 18.29 | 24.74 | 24.79 |
| | 4196.54 | | | Bovine | | | | | | |
| MW4300 | 4300.03/ | | 4 | Cytochrome C | N/A | 100.00 | 25.10 | 16.63 | 25.08 | 25.13 |
| | 4311.45 | | | | | | | | | |
| MW4821 | 4817.60/ | | 5 | | N/A | 100.00 | 26.56 | 17.11 | 26.54 | 26.59 |
| | 4823.26 | | | | | | | | | |
| MW8594 | 8590.93 | | 6 | Bovine | N/A | 85.71 | 35.44 | 16.95 | 35.42 | 35.47 |
| | | | | Cytochrome C | | | | | | |

| Definition of the cluster | | | | | | | | | | | | |
|---|---|---|---|---|---|---|---|---|---|---|---|---|
| | Mean mass | | | | | | | | | | | |
| | of the | | | | | | | | | | | |
| | substances | | | | | | | | | | | |
| | (calibration | | | | | | | | | | | |
| | according | | | | Signal quality | | | | | | | |
| | to bovine | | | | Signal-to-noise ratio | | | | | | | |
| | cytochrome | Annotation | Cluster | Calibrating | Min- | 25% | | 75% | | | Standard | Standard |
| Cluster | C) | mode | number | agent | imum | percentile | Median | percentile | Maximum | Mean | deviation | error |
| MW4152 | 4151.00/ | Automatic | 1 | Bovine | 2.50 | 20.87 | 38.47 | 67.56 | 179.62 | 55.92 | 52.20 | 9.23 |
| | 4163.88 | | | insulin | | | | | | | | |
| MW4168 | 4166.38/ | | 2 | (50% of the | 1.17 | 5.82 | 15.69 | 24.07 | 43.79 | 15.74 | 10.99 | 1.94 |
| | 4179.04 | | | spectral | | | | | | | | |
| MW4186 | 4184.41/ | | 3 | population)/ | 0.78 | 3.67 | 13.60 | 32.66 | 112.80 | 20.69 | 24.02 | 4.25 |
| | 4196.54 | | | Bovine | | | | | | | | |
| MW4300 | 4300.03/ | | 4 | Cytochrome C | 4.34 | 7.92 | 13.19 | 19.40 | 26.75 | 13.63 | 6.48 | 1.15 |
| | 4311.45 | | | | | | | | | | | |
| MW4821 | 4817.60/ | | 5 | | 7.06 | 15.08 | 18.00 | 25.15 | 35.94 | 19.66 | 7.13 | 1.26 |
| | 4823.26 | | | | | | | | | | | |
| MW8594 | 8590.93 | | 6 | Bovine | 1.04 | 4.39 | 6.44 | 11.81 | 25.56 | 8.27 | 5.02 | 0.89 |
| | | | | Cytochrome C | | | | | | | | |

TABLE 16
Principal study E6/Hydrophobic separation/Report regarding intensities/Automatic annotations/Normalization according to cluster MW6620
PARTIAL TABLE PRESENTED BY WAY OF EXAMPLE

| | | | | Control | | | | T21 | | | | T18 | | | |
|---|---|---|---|---|---|---|---|---|---|---|---|---|---|---|---|
| | | | | | Standard | | | | Standard | | | | Standard | | |
| Cluster | Cluster No. | TOF (μs) | Value p | Mean | deviation | Min | Max | Mean | deviation | Min | Max | Mean | deviation | Min | Max |
| MW3156 | 1 | 21.54 | 0.457208 | 4.45 | 4.12 | −0.46 | 13.73 | 5.90 | 5.95 | 1.04 | 22.23 | 2.56 | 1.68 | 0.99 | 4.49 |
| MW3215 | 2 | 21.73 | 0.009008 | 8.24 | 1.68 | 5.80 | 13.67 | 10.16 | 2.05 | 6.88 | 13.96 | 9.18 | 3.17 | 5.24 | 12.84 |
| MW4410 | 11 | 25.43 | 0.295773 | 5.68 | 5.58 | 2.56 | 27.60 | 5.37 | 3.09 | 2.63 | 14.80 | 4.41 | 1.47 | 3.16 | 6.53 |
| MW4463 | 12 | 25.59 | 0.179243 | 8.32 | 4.96 | 1.82 | 18.69 | 8.94 | 4.70 | 2.58 | 19.42 | 7.48 | 4.73 | 3.13 | 12.41 |
| MW4470 | 13 | 25.61 | 0.0982 | 9.37 | 4.13 | 3.04 | 16.95 | 10.98 | 6.06 | 4.37 | 29.35 | 9.05 | 3.32 | 5.07 | 11.99 |
| MW4650 | 14 | 26.11 | 0.103567 | 6.08 | 5.20 | 1.17 | 21.43 | 8.89 | 16.22 | 1.42 | 70.95 | 6.10 | 3.09 | 2.67 | 9.92 |
| MW5033 | 15 | 27.16 | 0.006125 | 2.99 | 2.11 | 0.71 | 8.97 | 4.47 | 8.59 | 0.86 | 38.29 | 3.49 | 1.47 | 2.04 | 5.53 |
| MW5625 | 16 | 28.70 | 0.044947 | 3.38 | 3.96 | 0.51 | 16.82 | 1.76 | 0.90 | 0.46 | 3.70 | 0.98 | 0.36 | 0.60 | 1.34 |
| MW5756 | 17 | 29.04 | 0.005933 | 8.41 | 3.26 | 4.09 | 16.07 | 10.91 | 7.23 | 2.71 | 33.55 | 8.96 | 6.19 | 5.05 | 18.13 |
| MW5799 | 18 | 29.15 | 0.010931 | 10.11 | 4.50 | 4.86 | 20.92 | 12.99 | 8.19 | 3.96 | 38.00 | 9.28 | 5.33 | 5.10 | 17.04 |
| MW6423 | 19 | 30.68 | 0.004728 | 13.35 | 2.05 | 9.74 | 19.41 | 16.51 | 2.88 | 12.15 | 24.46 | 14.32 | 4.01 | 9.41 | 19.21 |
| MW6439 | 20 | 30.72 | 0.188758 | 14.73 | 3.46 | 8.00 | 20.73 | 21.24 | 15.24 | 9.88 | 78.67 | 17.08 | 6.40 | 9.78 | 25.13 |
| MW6620 | 21 | 31.14 | 0.288472 | 28.31 | 0.07 | 28.24 | 28.57 | 28.29 | 0.03 | 28.23 | 28.38 | 28.29 | 0.01 | 28.27 | 28.30 |
| MW6636 | 22 | 31.18 | 0.552805 | 23.09 | 4.62 | 16.10 | 35.12 | 24.67 | 7.12 | 15.34 | 47.82 | 28.92 | 6.58 | 23.60 | 38.16 |
| MW6659 | 23 | 31.23 | 0.024619 | 7.35 | 1.08 | 5.52 | 9.41 | 8.82 | 2.31 | 5.71 | 15.44 | 9.75 | 1.38 | 8.64 | 11.76 |
| MW7020 | 24 | 32.07 | 0.063323 | 6.00 | 4.30 | 1.58 | 19.43 | 4.21 | 1.97 | 1.32 | 7.88 | 2.43 | 0.46 | 2.03 | 2.91 |
| MW8112 | 25 | 34.47 | 0.073538 | 1.52 | 1.12 | 0.05 | 3.45 | 3.64 | 7.49 | 0.14 | 32.68 | 2.27 | 2.33 | 0.05 | 4.44 |
| MW8563 | 26 | 35.41 | 0.012477 | 9.92 | 9.13 | 4.20 | 44.52 | 11.64 | 8.61 | 5.18 | 41.52 | 10.54 | 6.65 | 5.48 | 19.92 |
| MW8627 | 27 | 35.54 | 0.006125 | 16.10 | 5.60 | 8.25 | 30.85 | 22.41 | 14.89 | 10.09 | 73.99 | 17.95 | 11.28 | 10.78 | 34.71 |
| MW8691 | 28 | 35.67 | 0.012396 | 23.91 | 8.73 | 12.95 | 43.53 | 32.12 | 20.38 | 13.57 | 99.68 | 22.72 | 12.74 | 13.67 | 41.61 |
| MW8809 | 29 | 35.91 | 0.115651 | 8.15 | 3.41 | 5.16 | 20.22 | 10.57 | 6.68 | 5.19 | 33.76 | 7.45 | 3.03 | 4.82 | 11.81 |
| MW8929 | 30 | 36.15 | 0.010007 | 7.83 | 2.32 | 4.69 | 12.39 | 10.67 | 6.37 | 5.86 | 32.45 | 7.77 | 1.84 | 5.02 | 8.88 |
| MW9291 | 31 | 36.87 | 0.325234 | 4.34 | 2.26 | 1.75 | 11.24 | 6.11 | 8.90 | 1.70 | 40.62 | 3.89 | 1.59 | 2.18 | 5.70 |
| MW9360 | 32 | 37.01 | 0.170855 | 8.94 | 5.65 | 2.31 | 25.13 | 6.76 | 3.71 | 2.20 | 18.58 | 4.08 | 0.21 | 3.90 | 4.38 |
| MW14035 | 33 | 45.28 | 0.175758 | 4.79 | 3.58 | 0.67 | 14.43 | 3.04 | 1.71 | 0.62 | 6.05 | 1.61 | 0.35 | 1.14 | 1.97 |
| MW17262 | 34 | 50.19 | 0.008505 | 4.80 | 1.57 | 2.22 | 8.55 | 6.94 | 4.57 | 2.97 | 23.46 | 5.07 | 3.03 | 2.97 | 9.57 |
| MW17386 | 35 | 50.37 | 0.020274 | 5.77 | 2.17 | 2.71 | 10.50 | 8.12 | 5.11 | 3.28 | 26.12 | 5.41 | 2.98 | 3.40 | 9.83 |

### D-3 Spectra and Figures

### Profiles Obtained by Separation and Purification of Q10 Anionic Type—E6 Study:

FIG. 5 shows the spectra obtained on the 3000-20 000 dalton spectral zone, as overall views, for the control group, the T21 group and the T18 group. The normalized spectra are superimposed thereon per clinical category.

FIGS. 6, 7 and 8 show, respectively, the spectral views (spectra normalized and superimposed per clinical category), the pseudogel views and the spectra as shifted views (E6 study), centered on the 4000 Da zone.

FIGS. 9, 10 and 11 show, respectively, the spectral views (spectra normalized and superimposed per clinical category), the pseudogel views, and the spectra as shifted views (E6 study), centered on the 5000-6000 Da zone.

### Profiles Obtained by Separation of Q10 Anionic Type: E1 to E5

These profiles are shown on FIGS. 12 to 18, for the two clinical categories (controls and T21).

FIGS. 12 to 14 show the spectra centered on the 3000-10 000 dalton spectral zone, respectively as overall views (spectra normalized and superimposed per clinical category), pseudogel view, and shifted views (E1 to E5). FIG. 15 shows the spectra centered on the 3000-4000 dalton spectral zone (E1 to E5). FIGS. 16 to 18 show the spectra, normalized and superimposed per clinical category, centered respectively on the 4000-5000 dalton, 5000-7000 dalton and 7000-10 000 dalton zones (E1 to E5 studies).

### Profiles Obtained by H50 Separation According to the Hydrophobicity of the Proteins (E6 Study)

These profiles are presented in FIGS. 19 to 26.

### D-4 Examples of Distribution Statistics for the Intensities in the Groups

FIGS. 27 to 30 show examples of distribution statistics for the intensities of various clusters, for the control and T21 groups.

D-5 Examples of ROC Curves Constructed from the Areas Under the Curve for the Crude Intensities

The areas under the ROC (Receiver Operating Characteristic) curve for the peaks of interest, calculated from the crude intensities, are greater than 0.50, and can reach 0.95.

FIG. 31 shows the ROC curves for the molecular weight markers 3371, 3442, 3486 and 6423 Da.

### D-6 Example of Hierarchical Classification on Dataset E6 and Anionic

FIG. 32 shows the result of a construction with the Genecluster Version 1999 (Eisen M.) and Treeview Version 1.60 (Eisen M.) programs (hierarchical classification on dataset of the E6 study (CI 95%), Q10 anionic surface).

Construction method: Adjustment of the data by logarithmic transformation then normalization, in order, according to the spectra then the clusters. Then hierarchical clusterization simultaneously on the clusters and the spectra by Spearman correlation.

Results: Excellent discrimination of the control (“blue”), T21 (“red”), 7q deletion (series C), T18 (series E), series T21 −80° C. (“black”), series control −80° C. (“green”) groups.

### D-7 Example of Principal Component Analysis on Datasets of the E2 to E5 Studies and Anionic

The programs used are the Stata SE 8 program and the Ciphergen Express Version 1.0 program. The two programs confirm the results.

The actual values (or Eigen values) confirm the presence and the molecular weight of the peaks identified as being defensins, the masses of which are: MW3371, MW3442 and MW3486.

The results are shown in FIG. 33.

### D-8 Clinical Results and Applications for Screening

The results obtained for the peaks in the 3000 dalton zone, on a Q10 anionic surface, are presented in table 17. These three peaks are under-expressed or absent in the T21 group, reflecting a considerable reduction in immunity, and expressed normally or overexpressed in the control group. A more thorough study of the results will make it possible to determine the detection threshold for these three peaks, which are correlated and dependent, such that at least one negative sign among the three or one positive sign among the three signals the disease or the absence of disease, respectively. The results presented in table 17 exhibit a variability for these three peaks, mainly related to the fact that they were obtained with samples conserved at −20° C., exhibiting a certain amount of degradation.

Table 18 hereinafter presents the results and applications for the peaks in the 4000-9000 dalton zone, on a Q10 anionic surface.

Table 19 shows the results and applications for the peaks in the 4000-17 500 dalton zone, on an H50 hydrophobic surface.

TABLE 17
Selection of the samples/Anionic separating phase, pH > 4/
Clusters MW3371, MW3442 and MW3486

| | | Q10 accumulation E1-E5 | | |
|---|---|---|---|---|
| Samples | Group | MW3371 | MW3442 | MW3486 |
| 1 | DS | − | − | − |
| 2 | DS | − | − | − |
| 4 | DS | − | − | + |
| 5 | DS | − | − | − |
| 6 | DS | − | − | + |
| 7 | DS | − | − | − |
| 8 | DS | − | − | − |
| A1 | DS | − | − | − |
| A2 | DS | − | + | + |
| A2 | DS | + | + | + |
| A3 | DS | − | − | + |
| A4 | DS | + | + | − |
| A5 | DS | − | − | − |
| A6 | DS | − | − | − |
| A7 | DS | − | − | − |
| A8 | DS | − | − | − |
| 9 | Control | + | + | + |
| 10 | Control | − | + | + |
| 12 | Control | + | + | + |
| 13 | Control | + | − | + |
| 14 | Control | + | + | + |
| 15 | Control | + | + | + |
| 16 | Control | + | + | + |
| A9 | Control | − | − | − |
| A10 | Control | + | + | + |
| A10 | Control | + | + | + |
| A11 | Control | − | − | − |
| A12 | Control | − | − | − |
| A13 | Control | + | + | − |
| A14 | Control | + | + | + |
| A15 | Control | + | + | + |
| A16 | Control | − | − | + |

| | | | |
|---|---|---|---|
| Area under the ROC | 0.8164 | 0.8555 | 0.8164 |
| curve | | | |
| Standard error | 0.07572 | 0.06688 | 0.07419 |
| Confidence interval | 0.6680 to 0.9649 | 0.7244 to 0.9866 | 0.6710 to |
| (95%) | | | 0.9619 |
| Value P | 0.002279 | 0.0006092 | 0.002279 |

TABLE 18
Selection of the samples per clusters/Anionic separating phase, pH > 4/Some examples

| | | Q10 accumulation E1 to E5 | | | | | | |
|---|---|---|---|---|---|---|---|---|
| Samples | Groups | MW4152 | MW4168 | MW4186 | MW4201 | MW4300 | MW5695 | MW6222 |
| 1 | DS | − | − | − | − | − | + | + |
| 2 | DS | − | − | + | + | + | − | + |
| 3 | DS | N/A | N/A | N/A | N/A | N/A | N/A | N/A |
| 4 | DS | − | − | + | + | + | + | + |
| 5 | DS | + | + | − | − | + | − | + |
| 6 | DS | − | − | + | + | + | + | − |
| 7 | DS | − | − | + | + | + | + | − |
| 8 | DS | + | + | + | + | − | + | − |
| A1 | DS | + | + | − | − | + | + | − |
| A2 | DS | − | − | − | − | + | − | − |
| A2 | DS | + | + | − | − | − | + | − |
| A3 | DS | − | − | + | + | + | + | − |
| A4 | DS | + | + | + | + | + | − | − |
| A5 | DS | + | + | − | − | + | + | − |
| A6 | DS | + | + | − | − | + | + | − |
| A7 | DS | + | + | − | − | − | + | + |
| A7 | DS | N/A | N/A | N/A | N/A | N/A | N/A | N/A |
| A7 | DS | N/A | N/A | N/A | N/A | N/A | N/A | N/A |
| A8 | DS | + | + | + | + | + | − | + |
| 9 | Control | + | + | − | − | − | + | − |
| 10 | Control | + | + | − | − | + | − | − |
| 11 | Control | N/A | N/A | N/A | N/A | N/A | N/A | N/A |
| 12 | Control | + | + | − | − | + | + | − |
| 13 | Control | + | + | − | − | + | − | − |
| 14 | Control | − | | − | − | + | + | − |
| 15 | Control | + | + | + | + | − | + | − |
| 16 | Control | − | − | − | − | − | + | − |
| A9 | Control | + | + | − | − | + | − | − |
| A10 | Control | − | − | − | − | + | + | − |
| A10 | Control | + | + | − | − | − | + | − |
| A11 | Control | + | + | + | + | − | − | + |
| A12 | Control | + | + | − | − | + | − | − |
| A13 | Control | + | + | − | − | − | + | − |
| A14 | Control | + | + | + | + | − | + | − |
| A15 | Control | − | − | + | + | − | + | + |
| A15 | Control | N/A | N/A | N/A | N/A | N/A | N/A | N/A |
| A15 | Control | N/A | N/A | N/A | N/A | N/A | N/A | N/A |
| A16 | Control | − | − | + | + | + | − | + |
| A25 | Absent | N/A | N/A | N/A | N/A | N/A | N/A | N/A |
| | 80° C. | | | | | | | |
| A26 | Absent | N/A | N/A | N/A | N/A | N/A | N/A | N/A |
| | 80° C. | | | | | | | |
| A27 | Absent | N/A | N/A | N/A | N/A | N/A | N/A | N/A |
| | 80° C. | | | | | | | |
| A28 | Absent | N/A | N/A | N/A | N/A | N/A | N/A | N/A |
| | 80° C. | | | | | | | |
| Area under the ROC | | 0.5820 | 0.6055 | 0.6055 | 0.6758 | 0.5781 | 0.5820 | 0.5781 |
| curve | | | | | | | | |
| Standard error | | 0.1050 | 0.1029 | 0.1029 | 0.09707 | 0.1057 | 0.1042 | 0.1043 |
| Confidence interval | | 0.3761 to | 0.4037 to | 0.4037 to | 0.4855 to | 0.3710 to | 0.3777 to | 0.3736 to |
| (95%) | | 0.7880 | 0.8072 | 0.8072 | 0.8661 | 0.7853 | 0.7864 | 0.7826 |
| Value P | | 0.4287 | 0.3089 | 0.3089 | 0.08995 | 0.4510 | 0.4287 | 0.4510 |
| | | Principal study E6 | | | | | | |
| Samples | Groups | MW4152 | MW4168 | MW4186 | MW4201 | MW4300 | MW5695 | MW6222 |
| 1 | DS | + | − | + | − | + | + | + |
| 2 | DS | + | − | + | + | + | + | + |
| 3 | DS | + | + | + | + | + | − | + |
| 4 | DS | − | − | + | + | + | + | − |
| 5 | DS | + | + | − | − | + | − | + |
| 6 | DS | + | − | + | + | − | − | − |
| 7 | DS | + | + | + | + | + | + | + |
| 8 | DS | + | + | + | + | + | + | + |
| A1 | DS | + | + | − | − | + | − | − |
| A2 | DS | + | + | − | − | + | − | − |
| A2 | DS | N/A | N/A | N/A | N/A | N/A | N/A | N/A |
| A3 | DS | + | + | + | + | + | + | − |
| A4 | DS | + | + | + | + | + | − | − |
| A5 | DS | + | + | + | + | + | − | − |
| A6 | DS | + | + | + | − | + | − | + |
| A7 | DS | + | + | + | − | + | − | + |
| A7 | DS | + | + | + | − | + | − | + |
| A7 | DS | + | + | + | − | + | − | + |
| A8 | DS | + | − | + | + | − | − | + |
| 9 | Control | + | + | + | − | − | + | + |
| 10 | Control | + | + | + | − | + | + | − |
| 11 | Control | + | + | + | + | + | + | + |
| 12 | Control | + | + | + | − | + | + | + |
| 13 | Control | + | + | − | − | − | − | + |
| 14 | Control | + | + | + | + | − | + | + |
| 15 | Control | + | + | + | + | − | − | + |
| 16 | Control | + | − | − | − | + | + | + |
| A9 | Control | + | + | − | − | + | − | + |
| A10 | Control | + | + | + | − | + | − | + |
| A10 | Control | N/A | N/A | N/A | N/A | N/A | N/A | N/A |
| A11 | Control | + | + | − | + | + | + | + |
| A12 | Control | + | + | − | − | − | + | − |
| A13 | Control | + | + | + | − | − | − | + |
| A14 | Control | + | + | + | + | − | + | + |
| A15 | Control | + | + | + | + | − | − | + |
| A15 | Control | + | + | + | + | − | + | + |
| A15 | Control | + | + | + | + | + | + | + |
| A16 | Control | + | − | + | + | − | + | + |
| A25 | Absent | N/A | N/A | Absent | Absent | Present | Absent | Absent |
| | 80° C. | | | | | | | |
| A26 | Absent | Present | Absent | Present | Present | Absent | Absent | Absent |
| | 80° C. | | | | | | | |
| A27 | Absent | Present | Present | Absent | Absent | Present | Present | Absent |
| | 80° C. | | | | | | | |
| A28 | Absent | Present | Absent | Present | Present | Absent | Present | Absent |
| | 80° C. | | | | | | | |

| | | | | | | | |
|---|---|---|---|---|---|---|---|
| Area under the ROC | 0.5772 | 0.5401 | 0.5401 | 0.5648 | 0.6790 | 0.6543 | 0.6080 |
| curve | | | | | | | |
| Standard error | 0.09804 | 0.1003 | 0.09832 | 0.09735 | 0.09221 | 0.09370 | 0.09568 |
| Confidence interval | 0.3850 to | 0.3435 to | 0.3474 to | 0.3740 to | 0.4982 to | 0.4706 to | 0.4204 to |
| (95%) | 0.7694 | 0.7368 | 0.7329 | 0.7557 | 0.8598 | 0.8380 | 0.7956 |
| Value P | 0.4290 | 0.6809 | 0.6809 | 0.5065 | 0.06656 | 0.1137 | 0.2682 |

TABLE 19
Selection of the samples per clusters/Hydrophobic separating phase/Some examples

| Samples | Groups | MW4682 | MW5625 | MW6423# | MW6439# | MW6620 | MW6636# | MW9360 | MW14035 | MW17262 |
|---|---|---|---|---|---|---|---|---|---|---|
| 1 | DS | + | − | − | + | − | + | − | − | + |
| 2 | DS | − | − | + | + | − | − | − | + | + |
| 3* | DS | + | − | + | + | − | + | + | − | + |
| 4 | DS | − | + | + | − | + | − | − | − | − |
| 5 | DS | − | − | + | − | + | − | − | − | + |
| 6 | DS | − | − | − | − | + | − | − | − | + |
| 7 | DS | − | − | + | + | − | + | + | − | + |
| 8 | DS | − | + | − | − | + | − | − | + | + |
| A1* | DS | − | − | + | + | − | + | − | + | − |
| A2 | DS | − | − | + | + | − | + | − | + | + |
| A3 | DS | − | − | + | − | + | − | − | + | + |
| A4* | DS | − | − | + | − | − | − | − | + | + |
| A5 | DS | − | − | + | + | + | − | − | − | + |
| A6 | DS | − | + | + | − | − | − | + | − | + |
| A7 | DS | − | − | + | − | − | + | − | − | − |
| A7 | DS | − | − | + | + | + | + | − | − | + |
| A7 | DS | − | − | + | + | + | + | + | − | + |
| A8 | DS | − | − | + | + | − | + | − | + | + |
| 9 | Control | + | − | − | − | + | − | − | − | + |
| 10 | Control | + | + | − | − | − | − | + | + | − |
| 11* | Control | − | + | − | − | + | + | + | + | − |
| 12 | Control | + | + | − | − | + | − | + | + | + |
| 13 | Control | + | + | + | − | − | + | + | + | + |
| 14 | Control | − | + | − | + | + | − | + | + | + |
| 15 | Control | + | + | − | − | − | + | + | + | − |
| 16 | Control | + | + | − | − | + | − | + | + | − |
| A9* | Control | + | − | − | + | + | + | − | − | + |
| A10 | Control | − | − | − | − | + | − | − | − | + |
| A11 | Control | + | + | + | + | + | + | − | + | + |
| A12* | Control | + | + | − | + | − | + | + | − | − |
| A13 | Control | + | − | − | − | + | − | − | − | + |
| A14 | Control | + | + | − | − | + | − | + | + | − |
| A15 | Control | − | − | − | − | + | − | − | − | − |
| A15 | Control | − | − | − | − | + | + | − | − | − |
| A15 | Control | + | + | − | − | + | + | + | + | − |
| A16 | Control | + | + | − | − | + | + | + | + | + |
| A25 | −80° C. | Absent | Present | Present | Present | Present | Present | Present | Present | Present |
| A26 | −80° C. | Absent | Present | Present | Present | Present | Present | Present | Present | Present |
| A27 | −80° C. | Absent | Present | Present | Present | Present | Present | Present | Present | Present |
| A28 | −80° C. | Absent | Present | Present | Present | Present | Present | Present | Present | Present |

| | | | | | | | | | |
|---|---|---|---|---|---|---|---|---|---|
| Area under the ROC | 0.8133 | 0.7111 | 0.6000 | 0.6265 | 0.7111 | 0.8622 | 0.6756 | 0.7600 | 0.5644 |
| curve | | | | | | | | | |
| Standard error | 0.08624 | 0.09526 | 0.1076 | 0.09633 | 0.09793 | 0.06877 | 0.09850 | 0.08770 | 0.1089 |
| Confidence interval (95%) | 0.6443 to | 0.5244 to | 0.3891 to | 0.4377 to | 0.5191 to | 0.7274 to | 0.4824 to | 0.5881 to | 0.3510 to |
| | 0.9824 | 0.8979 | 0.8109 | 0.8154 | 0.9031 | 0.9970 | 0.8687 | 0.9319 | 0.7779 |
| Value P | 0.003469 | 0.04887 | 0.3507 | 0.1946 | 0.04887 | 0.0007292 | 0.1014 | 0.01528 | 0.5476 |

#Selective criteria obtained after normalization of the clusters relative to the intensity of cluster MW6620
*Samples used exclusively to test the selection criteria

### D-9 Purification and Identification: Some Examples

### Purification

Among various combinations tested—separation and purification surfaces, purification conditions, etc.—mention may be made of the following:

TABLE 20
Purification conditions/Some examples

| | 50 kDa < MW < 100 kDa | | | | | | MW > 100 kDa | | | | | | | |
|---|---|---|---|---|---|---|---|---|---|---|---|---|---|---|
| Separation | | | | Quaternary | | | | | | Quaternary | | | | |
| as a | | Hydrophobic | | amine | | | | Hydrophobic | | amine | | | Crude serum | |
| function of | | C₆-C₁₈ | Targeted | | | Physio- | | C₆-C₁₈ | Targeted | | | Physio- | Targeted | |
| native mass | | Physiological | approach | | | logical | | Physiological | approach | | | logical | approach | |
| Separation | Silicon | pH and | Gallium | pH | pH | pH and | Silicon | pH and | Gallium | pH | pH | pH and | | PAPP- |
| conditions | oxide | conditions | ion | 4 | 8 | conditions | oxide | conditions | ion | 4 | 8 | conditions | hCG | A |
| MW3371 | − | − | + | + | + | + | − | − | − | − | − | − | − | − |
| MW3442 | − | − | + | + | + | + | − | − | − | − | − | − | − | − |
| MW3486 | − | − | + | + | + | + | − | − | − | − | − | − | − | − |
| MW6423 | −* | + | − | − | −* | −* | + | + | − | − | − | − | + | + |
| MW6439 | −* | + | − | − | −* | −* | + | + | − | − | − | − | + | + |
| MW6620 | −* | + | − | − | −* | −* | + | + | − | − | − | − | + | + |
| MW6636 | −* | + | − | − | −* | −* | + | + | − | − | − | − | + | + |

−*: Signal greatly disturbed by the massive presence of albumin. Experiments after depletion of albumin are ongoing.

### Identification of Clusters MW3371, MW3442 and MW3486

### Considerations Regarding the Native Mass

These three peptides have correlated biophysicochemical properties. They are present in a native fraction of the samples such that the mass of the products is between 50 and 100 kDa or greater than 100 kDa. They are therefore peptides associated with one or more proteins, such that the mass of the complexes thus formed is greater than 50 kDa. A targeted approach made it possible to identify a possible association of HNPs with: albumin, α-fetoprotein and/or immunoglobulins G.

### Considerations Regarding Phosphorylation

These peptides are found in a targeted approach using gallium as affinity molecule. Since the affinity of phosphorylated amino acids for gallium (Posewitz M C, Tempst P., Immobilized gallium(III) affinity chromatography of phosphopeptides. Anal Chem. 1999 Jul. 15; 71(14): 2883-92.) is recognized, it is therefore possible to put forward the hypothesis that either these peptides, or one of the molecules that are associated with them, has one or more phosphorylated amino acids.

### Considerations Regarding the Differences in Masses and Possible Identification

This involves considerations regarding the difference in mass between two of the three peptides.
MW3442−MW3371=71 Da→alanine (A)
MW3486−MW3371=115 Da→aspartic acid (D)
MW3486−MW3442=44 Da→replacement of an aspartic acid (D) with an alanine (A).

Derived from the data banks, only the sequences of the human neutrophil defensins (or HNPs) correspond to the stated criteria (see table 21).

TABLE 21

Amino acid sequence of the α-Defensins
(HNP-1, HNP-2, HNP-3)

| | | Molecular | |
|---|---|---|---|
| | | Weights | Difference |
| Names | Amino acid sequence | (Da) | with HNP-2 |
| Human | -CYCRIPACIAGERRYGTCIYQGRLWAFCC | 3371.01 | — |
| neutrophil | | | |
| Defensin 2 | | | |
| Human | [symbol] CYCRIPACIAGERRYGTCIYQGRLWAFCC | 3442.09 | A |
| neutrophil | | | |
| Defensin 1 | | | |
| Human | | | |
| neutrophil | [symbol] CYCRIPACIAGERRYGTCIYQGRLWAFCC | 3486.10 | D |
| Defensin 3 | | | |

### Identification of Clusters MW6423, MW6620, and MW6636

### Considerations Regarding the Native Mass

These four peptides have correlated biophysicochemical properties. They are present in a native fraction of the samples such that the mass of the products is between 50 and 100 kDa, and in the native mass fraction greater than 100 kDa. They are therefore peptides associated with one or more proteins, such that the mass of the complexes thus formed is between 50 and 100 kDa, or greater than 100 kDa.

### Consideration Regarding the Differences in Masses and Possible Identification

This involves considerations regarding the difference in mass between two of the four peptides.
MW6636−MW6620=16 Da [symbol] possible hydrolysis of one proline in hydroxyproline
MW6636−MW6439=197 Da±1 Da [symbol] Valine+Proline (196 kDa) or Proline+Threonine (198 kDa)
MW6636−MW6423=181 Da±1 Da [symbol] no combination between amino acids
MW6620−MW6439=181 Da±1 Da [symbol] no combination between amino acids
MW6620−MW6423=197 Da±1 Da [symbol] Valine+Proline (196 kDa) or Proline+Threonine (198 kDa)
MW6439−MW6423=16 Da [symbol] possible hydrolysis of one proline in hydroxyproline

These considerations are summed up in FIG. 34.

### E—Conclusions Regarding the First Series of Studies

The results relating to 19 discriminating markers of the Control and T21 populations are presented above. All the markers presented in table 14 can be included in combinations with one another or in combination with the triple test or else with one or two markers of the triple test. These markers, which are overexpressed or underexpressed, in the control group or in the sick group, are all of diagnostic interest.

In particular, the peaks of mass MW3371, MW3442 and MW3486, identified as defensins, are underexpressed in the T21 group and overexpressed or normally expressed in the controls.

The peaks of mass MW6423, MW6439, MW6620 and MW6636 can be identified in pairs with a hydrolysis of a proline to hydroxyproline (+16 Da).

### F—Supplementary Studies on the Use of Defensins as Diagnostic Markers

Supplementary studies aimed at confirming the relevance of the defensins HNP-2, HNP-1 and HNP-3 as prenatal diagnostic markers of Down syndrome were carried out. Various experimental conditions were used. In particular, the inventors varied the working pH and tested various fractionation conditions (fraction C or native serum sample).

These studies showed that, although the defensins can theoretically be visualized at pH=7.4, they are, in reality, only detectable at a pH of less than 5. This is probably due to the fact that at least some of these defensins are complexed with alpha-feto-protein and/or with albumin.

Moreover, the inventors calculated the longitudinal ratios (i.e. between 2 peaks of the same spectrum) between the intensities of the peaks corresponding to the various HNPs. Notably, they observed, only under the native serum condition, a significant difference in these ratios, depending on whether the sample is from a woman carrying a fetus suffering from trisomie 21 or from a woman carrying a normal fetus.

The results of three of these studies are presented hereinafter, and also in FIGS. 35 to 38. The studies of which the results are presented in detail were carried out based on three sets of data (datasets). They are identified as follows:
NS502: Q10 anionic purification/native serum/pH=4/−80° C., using the samples listed in table 22 hereinafter,
NS503: idem NS503, but pH=5, and
NS403CDsf: Q10 anionic purification/serum fraction C/pH=4, using the samples listed in table 23 hereinafter.

TABLE 22
DATASET NS502 POPULATION TESTED (Q10/anion exchange/native/pH 4/−80° C.)

| Sample | | | | | | | | | | | | | | | | Other |
|---|---|---|---|---|---|---|---|---|---|---|---|---|---|---|---|---|
| No. | Sampling date | Status | Age | Weight | Wk + d | hcg_iu | MoMhcg | ue3_pg | MoMue3 | afp_iu | MoMafp | Risk | Karyotype | | Outcome | information |
| F | 20 11 02 | CTRL | 27 | | 15 + 0 | 26.6 | 0.83 | 860 | 1.42 | 21.8 | 0.87 | 1/10000 | | | | girl H |
| F14 | 12 03 03 | CTRL | 39 | 74 | 15 + 5 | 25.60 | 1.04 | 640 | 0.86 | 22.70 | 0.87 | 1/320 | 46, XX | age | | girl H |
| F4 | 22 03 04 | CTRL | 34 | | 14 + 4 | 16 | 0.43 | 650 | 1.27 | 24.4 | 1.00 | 1/8400 | 45, XX, der(13; 14) | | | girl H |
| F5 | 30 03 04 | CTRL | 27 | | 14 + 2 | 27.2 | 0.67 | 350 | 0.79 | 17.7 | 0.77 | 1/3300 | | | | girl H |
| M10 | 26/11/2004 | CTRL | 35.00 | 46 | 16 + 1 | 43.90 | 1.57 | 720.0 | 0.74 | 36.10 | 1.00 | 241 | 46, XX | | | girl H |
| M11 | 27/11/2004 | CTRL | 32.00 | 62 | 16 + 4 | 58.80 | 2.65 | 1010.0 | 1.00 | 50.20 | 1.53 | 550 | | | | girl H |
| M12 | 27/11/2004 | CTRL | 38.00 | 52 | 15 + 4 | 19.00 | 0.61 | 470.0 | 0.59 | 25.10 | 0.80 | 242 | 46, XX | | | girl H |
| M13 | 27/11/2004 | CTRL | 30.00 | 54 | 14 | 16.20 | 0.33 | 410.0 | 1.12 | 22.90 | 1.00 | 10000 | | | | girl H |
| M14 | 28/11/2004 | CTRL | 28.00 | 79 | 15 + 6 | 59.70 | 2.65 | 780.0 | 1.03 | 22.20 | 0.88 | 420 | | | | girl H |
| M15 | 28/11/2004 | CTRL | 36.00 | 57 | 16 + 2 | 10.40 | 0.42 | 690.0 | 0.72 | 23.60 | 0.71 | 1100 | | | | boy H |
| M16 | 27/11/2004 | CTRL | 29.00 | | 16 + 2 | 44.50 | 1.95 | 520.0 | 0.55 | 29.20 | 0.91 | 86 | 46, XY | | | boy H |
| M17 | 27/11/2004 | CTRL | 24.00 | 59 | 17 | 13.50 | 0.65 | 1340.0 | 1.18 | 41.30 | 1.15 | 10000 | | | | girl H |
| M18 | 27/11/2004 | CTRL | 30.00 | 52 | 16 + 6 | 20.30 | 0.89 | 1340.0 | 1.18 | 29.10 | 0.77 | 6500 | | | | boy H |
| M19 | 26/11/2004 | CTRL | 32.00 | 74 | 17 + 2 | 13.40 | 0.77 | 1340.0 | 1.19 | 39.50 | 1.22 | 10000 | | | | girl H |
| M5 | 26/11/2004 | CTRL | 25.00 | | 17 + 4 | 11.30 | 0.64 | 1190.0 | 0.94 | 46.30 | 1.22 | 10000 | | | | girl H |
| M7 | 26/11/2004 | CTRL | 25.00 | 58 | 17 + 1 | 18.50 | 0.91 | 550.0 | 0.47 | 40.90 | 1.11 | 670 | | | | girl H |
| M8 | 26/11/2004 | CTRL | 29.00 | | 21 + 2 | 13.60 | 1.05 | 3710.0 | 1.68 | 93.10 | 1.69 | 0 | | | | boy H |
| M9 | 26/11/2004 | CTRL | 28.00 | 71 | 15 + 5 | 20.70 | 0.82 | 700.0 | 0.92 | 28.10 | 1.05 | 8200 | | | | boy H |
| F1 | 18 02 04 | DS | 32 | | 14 + 6 | 109.8 | 3.28 | 310 | 0.54 | 13.1 | 0.51 | 1/5 | 47, XY, +21 | markers | MIP | disformity |
| F10 | 23 03 04 | DS | 39 | | 15 + 0 | 101.8 | 3.21 | 580 | 0.94 | 18.3 | 0.70 | 1/21 | 47, XY, +21 | age | MIP | |
| F11 | 25 03 04 | DS | 40 | | 16 + 1 | 16.8 | 0.71 | 620 | 0.68 | 24.8 | 0.79 | 1/235 | 47, XX, +21 | age | MIP | |
| F12 | 02 04 04 | DS | 23 | | 16 + 2 | 67.5 | 2.95 | 600 | 0.64 | 33.4 | 1.04 | 1/71 | 47, XY, +21 | nuchal | MIP | |
| | | | | | | | | | | | | | | translucency | | |
| F13 | 29 04 04 | DS | 32 | | 15 + 0 | 57.9 | 1.82 | 420 | 0.68 | 11.1 | 0.42 | 1/40 | 47, XX, +21 | nuchal | MIP | |
| | | | | | | | | | | | | | | translucency | | |
| F2 | 28 01 04 | DS | 27 | | 15 + 1 | 67.9 | 2.24 | 400 | 0.63 | 18.5 | 0.73 | 1/68 | 47, XX, +21 | markers | MIP | disformity |
| F3 | 30 03 04 | DS | 28 | | 17 + 5 | 48.3 | 2.8 | 33 | 0.02 | 51.77 | 1.34 | 1/41 | 46, XX, der(14; 21) | 1(14; 21)mat | MIP | no anat path. |
| F6 | 02 05 04 | DS | 34 | | 14 + 2 | 209.4 | 5.18 | 490 | 1.12 | 18.3 | 0.79 | 1/91 | 47, XX, +21 | hygroma | MIP girl | |
| F7 | 09 03 04 | DS | 39 | | 16 + 4 | 92.3 | 4.29 | 810 | 0.79 | 31.8 | 0.96 | 1/5 | 47, XX, +21 | age | MIP | |
| F8 | 16 03 04 | DS | 34 | 65 | 17 + 3 | 84 | 4.59 | 1040 | 0.86 | 22.3 | 0.62 | 1/5 | 47, XY, +21 | markers | MIP | |
| F9 | 22 03 04 | DS | 32 | 60 | 20 + 0 | 30.6 | 2.12 | 1380 | 0.72 | 36.6 | 0.72 | 1/81 | 47, XY, +21 | markers | MIP | |
| M1 | 28/09/2004 | DS | 31 | | 20 | 36.8 | 2.69 | 2870 | 1.5 | 115.8 | 2.36 | 2800 | 47, XY, +21 | | MIP | |
| M2 | 15/12/2004 | DS | 38.00 | | 17 + 3 | 48.70 | 2.68 | 650.0 | 0.52 | 42.80 | 1.15 | 8 | 47, XX, +21 | | MIP | |
| M3 | 06/01/2005 | DS | 44.00 | 69 | 17 + 2 | 42.90 | 2.37 | 910.0 | 0.79 | 22.00 | 0.65 | 5 | 47, XX, +21 | | MIP | |
| M6 | 26/11/2004 | DS | 31.00 | 81 | 16 + 3 | 27.30 | 1.41 | 870.0 | 0.98 | 13.20 | 0.49 | 480 | 47, XX, +21 | short femur | MIP | |

TABLE 23
DATASET NS403CDsf POPULATION TESTED (Q10/anion exchange/fraction C/pH = 4)

| | Sample | | Samp. | | | | | | | | |
|---|---|---|---|---|---|---|---|---|---|---|---|
| Group | No. | Hosp Code | date | Age | Weight | Week | Day | Gestation D | hCG IU | Hcg MoM | uE3 pg |
| CTRL | 9 | 41 284 014 | 12 07 01 | 33 | — | 16 | 1 | 113 | 37.3 | 1.55 | 740 |
| CTRL | 10 | 41 281 019 | 09 07 01 | 30 | 78 | 15 | 3 | 108 | 41.8 | 1.58 | 550 |
| CTRL | 12 | 41 205 063 | 16 05 01 | 28 | 66 | 15 | 0 | 105 | 40.3 | 1.22 | 520 |
| CTRL | 13 | 41 125 034 | 23 03 01 | 26 | 57 | 16 | 4 | 109 | 72.1 | 2.37 | 450 |
| CTRL | 14 | 41 276 009 | 07 07 01 | 28 | 61 | 16 | 0 | 112 | 57.5 | 2.24 | 590 |
| CTRL | 15 | 41 262 046 | 26 06 01 | 22 | 60 | 17 | 1 | 120 | 37.2 | 1.76 | 810 |
| CTRL | 16 | 41 174 040 | 26 04 01 | 33 | 53 | 17 | 1 | 120 | 37.8 | 1.68 | 1060 |
| CTRL | A9 | 119933 | 19/01/99 | 33 | — | 16 | 4 | 116 | 31.50 | 1.46 | 900.00 |
| CTRL | A10 | 49494010 | 07/12/99 | 33 | — | 16 | 3 | 115 | 26.50 | 1.20 | 620.00 |
| CTRL | A11 | 113591 | 28/07/98 | 29 | — | 16 | 3 | 115 | 33.60 | 1.51 | 580.00 |
| CTRL | A12 | 116833 | 27/10/99 | 27 | — | 15 | 2 | 107 | 39.70 | 1.34 | 220.00 |
| CTRL | A13 | 41084032 | 22/02/01 | 36 | — | 14 | 6 | 104 | 71.10 | 1.97 | 240.00 |
| CTRL | A14 | 113742 | 28/07/98 | 34 | — | 15 | 2 | 107 | 43.10 | 1.36 | 370.00 |
| CTRL | A15 | 113512 | 22/07/98 | 38 | — | 17 | 5 | 124 | 31.40 | 1.80 | 1 240 |
| CTRL | A16 | 40022037 | 07/01/00 | 38 | — | 15 | 2 | 107 | 57.20 | 1.87 | 570.00 |
| DS | 1 | 40 233 027 | 07 06 00 | 35 | — | 16 | 1 | 113 | 96.5 | 4.02 | 540 |
| DS | 2 | 40 351 013 | 28 08 00 | 27 | 56 | 15 | 5 | 110 | 118.2 | 4.02 | 300 |
| DS | 4 | 41 282 050 | 10 07 01 | 32 | 52 | 15 | 0 | 105 | 82.4 | 2.22 | 750 |
| DS | 5 | 42 114 001 | 14 03 02 | 31 | 57 | 15 | 4 | 109 | 67.6 | 2.22 | 440 |
| DS | 6 | 42 041 032 | 21 01 02 | 29 | 54 | 15 | 6 | 111 | 68.9 | 2.41 | 500 |
| DS | 7 | 40 364 001 | 07 09 00 | 36 | — | 17 | 1 | 120 | 80.2 | 3.99 | 450 |
| DS | 8 | 42 394 032 | 25 09 02 | 21 | 78 | 16 | 4 | 116 | 51.3 | 2.57 | 560 |
| DS | A1 | 119929 | 22/01/99 | 28 | — | 16 | 3 | 115 | 35.70 | 1.61 | 810.00 |
| DS | A2 | 49494032 | 08/12/99 | 36 | — | 16 | 4 | 116 | 24.00 | 1.11 | 680.00 |
| DS | A3 | 114172 | 14/08/98 | 30 | — | 15 | 4 | 109 | 95.00 | 3.46 | 400.00 |
| DS | A4 | 116783 | 27/10/98 | 31 | — | 16 | 4 | 116 | 34.00 | 1.40 | 290.00 |
| DS | A5 | 41153015 | 07/04/01 | 34 | 65 | 16 | 3 | 115 | 88.90 | 3.87 | 560.00 |
| DS | A6 | 113608 | 28/07/98 | 30 | — | 16 | 1 | 113 | 58.00 | 2.39 | 480.00 |
| DS | A7 | 40411009 | 01/09/00 | 38 | — | 17 | 2 | 121 | 25.30 | 1.29 | 1 390 |
| DS | A8 | 40526003 | 21/12/00 | 40 | — | 17 | 9 | 128 | 37.70 | 1.84 | 810.00 |

| | Sample | | | | | | | | |
|---|---|---|---|---|---|---|---|---|---|
| Group | No. | uE3 | AFP IU | AFP Mom | Risk R4 | sex | Outcome | Karyotype | Anat. path. |
| CTRL | 9 | 0.87 | 11.1 | 0.35 | 1/90 | M | | 46, XY | |
| CTRL | 10 | 0.89 | 23.6 | 0.99 | 1/970 | M | | | |
| CTRL | 12 | 0.94 | 20.1 | 0.80 | 1/1900 | M | | | |
| CTRL | 13 | 0.63 | 30.8 | 1.03 | 1/116 | F | | 46, XX | |
| CTRL | 14 | 0.72 | 20.3 | 0.66 | 1/95 | F | | 46, XX | |
| CTRL | 15 | 0.74 | 19.5 | 0.54 | 1/182 | F | | 46, XX | |
| CTRL | 16 | 0.94 | 33.6 | 0.87 | 1/510 | F | | | |
| CTRL | A9 | 0.88 | 24.90 | 0.74 | 1/332 | F | — | | girl H |
| CTRL | A10 | 0.63 | 30.20 | 0.92 | 1/474 | M | — | | |
| CTRL | A11 | 0.59 | 30.67 | 0.94 | 1/254 | F | — | 46, XX | |
| CTRL | A12 | 0.32 | 29.40 | 1.06 | 1/431 | M | | 46, XY | boy H |
| CTRL | A13 | 0.45 | 21.80 | 0.86 | 1/11 | F | — | 46, XX | girl H |
| CTRL | A14 | 0.60 | 18.08 | 0.70 | 1/76 | M | — | 46, XY | boy H |
| CTRL | A15 | 0.94 | 52.95 | 1.37 | 1/239 | F | — | 46, XX | girl H |
| CTRL | A16 | 0.93 | 42.15 | 1.53 | 1/166 | M | — | 46, XY | boy H |
| DS | 1 | 0.70 | 13.17 | 0.42 | 1/10 | M | MIP | 47, XY, +21 | no malformation |
| DS | 2 | 0.42 | 32.9 | 1.07 | 1/8 | M | MIP | 47, XY, +21 | nuchal edema |
| DS | 4 | 1.25 | 21.9 | 0.77 | 1/660 | M | MIP | 47, XY, +21 | tetralogy of failot |
| DS | 5 | 0.62 | 39.5 | 1.32 | 1/126 | F | MIP | 47, XX, +21 | nuchal edema |
| DS | 6 | 0.62 | 33.9 | 1.06 | 1/84 | F | MIP | 47, XX, +21 | membranous VSD |
| DS | 7 | 0.46 | 40.2 | 1.12 | 1/5 | F | MIP | 47, XX, +21 | VSD |
| DS | 8 | 0.60 | 36.2 | 1.28 | 1/143 | F | MIP | 47, XX, +21 | no malformation |
| DS | A1 | 0.82 | 23.38 | 0.71 | 1/441 | F | | 47, XX, +21 | born |
| DS | A2 | 0.66 | 23.30 | 0.68 | 1/153 | M | | 47, XY, +21 | born (VSD-ASD) |
| DS | A3 | 0.52 | 38.40 | 1.33 | 1/17 | F | MIP | 47, XX, +21 | |
| DS | A4 | 0.31 | 18.70 | 0.56 | 1/37 | M | MIP | 47, XY, +21 | |
| DS | A5 | 0.63 | 15.10 | 0.48 | 1/5 | F | MIP | 47, XX, +21 | |
| DS | A6 | 0.55 | 11.00 | 0.39 | 1/10 | M | MIP | 47, XY, +21 | |
| DS | A7 | 1.26 | 22.40 | 0.61 | 1/310 | F | MIP | 47, XX, +21 | |
| DS | A8 | 0.76 | 28.80 | 0.81 | 1/32 | M | MIP | 47, XY, +21 | |

H = healthy

The results of these studies are presented in tables 24 to 29 hereinafter, in which the following abbreviations and/or terms are used: CI=confidence interval; Dataset; Obs.=number of samples observed; CG=control group; DS or Down S.=Down syndrome; Std. Err.=standard error; SN=signal-to-noise; Mean; AUC=area under curve; var AUC=variance of AUCs; pAUC (8%)=partial area under curve, a rate of false positives (FP) of 8% was imposed on the Dataset.

TABLE 24
HNP descriptive statistics

| | |
|---|---|
| Obs. | MW (Substance Mass) |

| | | | | | | | | |
|---|---|---|---|---|---|---|---|---|
| Date | Condition | Dataset | n = CG | n = DS | Cluster No. | Mean | Std. Err. | [95% CI] |

HNP2

| | | | | | | | | |
|---|---|---|---|---|---|---|---|---|
| 2005 | Native, pH5, −80 | NS503 (N = 33) | 18 | 15 | 1 | 3374.70 | 0.24 | (3374.21-3375.18) |
| 2005 | Native, pH4, −80 | NS502 (N = 33) | 18 | 15 | 3 | 3375.89 | 0.23 | (3375.42-3376.35) |
| 2003 CD | Fraction C, pH4 | NS403 (N = 30) | 15 | 15 | 1 | 3389.80 | 0.44 | (3388.90-3390.71) |

HNP1

| | | | | | | | | |
|---|---|---|---|---|---|---|---|---|
| 2005 | Native pH5 −80 | NS503 (N = 33) | 18 | 15 | 2 | 3445.54 | 0.31 | (3444.91-3446.17) |
| 2005 | Native pH4 −80 | NS502 (N = 33) | 18 | 15 | 4 | 3447.13 | 0.22 | (3446.68-3447.58) |
| 2003 CD | Fraction C pH4 | NS403 (N = 30) | 15 | 15 | 2 | 3461.03 | 0.43 | (3460.14-3461.91) |

HNP3

| | | | | | | | | |
|---|---|---|---|---|---|---|---|---|
| 2005 | Native pH5 −80 | NS503 (N = 33) | 18 | 15 | 3 | 3489.01 | 0.41 | (3488.18-3489.83) |
| 2005 | Native pH4 −80 | NS502 (N = 33) | 18 | 15 | 5 | 3489.94 | 0.35 | (3489.24-3490.65) |
| 2003 CDsf | Fraction C pH4 | NS403 (N = 30) | 15 | 15 | 3 | 3503.77 | 0.44 | (3502.87-3504.66) |

| | | | | | | | | | | Median |
|---|---|---|---|---|---|---|---|---|---|---|
| | | | Obs. | | | Mean intensity of the cluster | | | | test |
| Date | Condition | Dataset | n = CG | n = DS | Cluster No. | Mean CG | [95% CI] | Mean DS | [95% CI] | p value |
| HNP2 | | | | | | | | | | |
| 2005 | Native pH5 −80 | NS503 (N = 33) | 18 | 15 | 1 | 2.88 | (1.53-4.22) | 2.23 | (1.25-3.21) | 1 |
| 2005 | Native pH4 −80 | NS502 (N = 33) | 18 | 15 | 3 | 9.74 | (6.60-12.87) | 7.26 | (5.12-9.41) | 0.373 |
| 2003 CD | Fraction C pH4 | NS403 (N = 30) | 15 | 15 | 1 | 9.76 | (3.67-15.85) | 2.90 | (2.05-3.74) | 0.01 |
| HNP1 | | | | | | | | | | |
| 2005 | Native pH5 −80 | NS503 (N = 33) | 18 | 15 | 2 | 3.59 | (1.76-5.42) | 2.54 | (1.06-4.02) | 0.169 |
| 2005 | Native pH4 −80 | NS502 (N = 33) | 18 | 15 | 4 | 8.92 | (5.29-12.56) | 6.52 | (3.64-9.39) | 0.04 |
| 2003 CD | Fraction C pH4 | NS403 (N = 30) | 15 | 15 | 2 | 10.65 | (4.33-16.97) | 3.14 | (2.11-4.18) | 0.001 |
| HNP3 | | | | | | | | | | |
| 2005 | Native pH5 −80 | NS503 (N = 33) | 18 | 15 | 3 | 2.91 | (0.60-5.23) | 1.09 | (0.55-1.63) | 0.492 |
| 2005 | Native pH4 −80 | NS502 (N = 33) | 18 | 15 | 5 | 5.99 | (2.96-9.02) | 2.33 | (1.25-3.41) | 0.112 |
| 2003 CDsf | Fraction C pH4 | NS403 (N = 30) | 15 | 15 | 3 | 9.96 | (3.87-16.06) | 2.86 | (1.86-3.85) | 0.060 |
| | | | | | | | | | | Median |
| | | | Obs. | | | Mean of the cluster (SN) | | | | test |
| Date | Condition | Dataset | n = CG | n = DS | Cluster No. | Mean CG | [95% CI] | Mean DS | [95% CI] | p value |
| HNP2 | | | | | | | | | | |
| 2005 | Native pH5 −80 | NS503 (N = 33) | 18 | 15 | 1 | 2.65 | (1.73-3.57) | 2.36 | (1.44-3.28) | 0.49 |
| 2005 | Native pH4 −80 | NS502 (N = 33) | 18 | 15 | 3 | 16.36 | (11.50-21.23) | 13.28 | (9.83-16.73) | 0.025 |
| 2003 CD | Fraction C pH4 | NS403 (N = 30) | 15 | 15 | 1 | 15.47 | (3.24-27.69) | 4.08 | (2.98-5.18) | 0.06 |
| HNP1 | | | | | | | | | | |
| 2005 | Native pH5 −80 | NS503 (N = 33) | 18 | 15 | 2 | 3.37 | (1.92-4.82) | 2.59 | (1.22-3.96) | 0.49 |
| 2005 | Native pH4 −80 | NS502 (N = 33) | 18 | 15 | 4 | 14.84 | (9.47-20.21) | 11.65 | (6.72-16.57) | 0.04 |
| 2003 CD | Fraction C pH4 | NS403 (N = 30) | 15 | 15 | 2 | 16.90 | (4.08-29.73) | 4.34 | (2.98-5.70) | 0.06 |
| HNP3 | | | | | | | | | | |
| 2005 | Native pH5 −80 | NS503 (N = 33) | 18 | 15 | 3 | 2.48 | (0.88-4.07) | 1.16 | (0.61-1.70) | 0.49 |
| 2005 | Native pH4 −80 | NS502 (N = 33) | 18 | 15 | 5 | 9.75 | (5.00-14.50) | 4.24 | (2.16-6.31) | 0.112 |
| 2003 CDsf | Fraction C pH4 | NS403 N = 30 | 15 | 15 | 3 | 15.52 | (3.65-27.38) | 3.90 | (2.76-5.05) | 0.06 |

TABLE 25
AUC Estimations (continuous data and nonparametric estimation)

| Date | Condition | Dataset | CG | DOWN S. | Cluster No. | m/z | DS | Auc (10) | Var (auc) | pAuc (8%) |
|---|---|---|---|---|---|---|---|---|---|---|
| AUC Empirical estimation (Variable: mass/charge) (m/z) | | | | | HNP2 | | | | | |
| 2005 | Native pH5 −80 | NS503 (N = 33) | 18 | 15 | 1 | 3374.70 | 1.40 | 0.55 | 0.00 | 0.47 |
| 2005 | Native pH4 −80 | NS502 (N = 33) | 18 | 15 | 3 | 3375.90 | 1.30 | 0.51 | 0.00 | 0.43 |
| 2003 CDsf | Fraction C pH4 | NS403 (N = 30) | 15 | 15 | 1 | 3389.80 | 2.40 | 0.64 | 0.00 | 0.56 |
| AUC Empirical estimation (Variable: Mass/charge) (m/z) | | | | | HNP1 | | | | | |
| 2005 | Native pH5 −80 | NS503 (N = 33) | 18 | 15 | 2 | 3445.50 | 1.80 | 0.56 | 0.00 | 0.48 |
| 2005 | Native pH4 −80 | NS502 (N = 33) | 18 | 15 | 4 | 3447.10 | 1.30 | 0.62 | 0.01 | 0.54 |
| 2003 CDsf | Fraction C pH4 | NS403 (N = 30) | 15 | 15 | 2 | 3461.00 | 2.40 | 0.72 | 0.01 | 0.64 |
| AUC Empirical estimation (Variable: Mass/charge) (m/z) | | | | | HNP3 | | | | | |
| 2005 | Native pH5 −80 | NS503 (N = 33) | 18 | 15 | 3 | 3489.00 | 2.40 | 0.58 | 0.00 | 0.50 |
| 2005 | Native pH4 −80 | NS502 (N = 33) | 18 | 15 | 5 | 3489.90 | 2.00 | 0.67 | 0.01 | 0.59 |
| 2003 CDsf | Fraction C pH4 | NS403 (N = 30) | 15 | 15 | 3 | 3503.80 | 2.40 | 0.71 | 0.01 | 0.63 |

TABLE 26
Descriptive statistics ratios

| | | | | | | | Ratio of the mean intensities | | | | |
|---|---|---|---|---|---|---|---|---|---|---|---|
| | | | Obs. | | Mean | Std. | | Mean | | | |
| Date | Condition | Dataset | n = CG | n = DS | CG | Error | [95% CI] | DS | Std. Error | [95% CI] | p Mann_Whitney |

Ratio ⅓

| | | | | | | | | | | | |
|---|---|---|---|---|---|---|---|---|---|---|---|
| 2005 | Native pH4 −80 | NS502 (N = 33) | 18 | 15 | 2.77 | 0.91 | (0.84-4.69) | 3.87 | 0.70 | (2.37-5.37) | 0.01 |
| 2005 | Native pH5 −80 | NS503 (N = 33) | 18 | 15 | 2.06 | 0.42 | (1.17-2.95) | 2.11 | 0.31 | (1.45-2.77) | 0.35 |
| 2003 CDsf | Fraction C pH4 | NS403 (N = 30) | 15 | 15 | 1.13 | 0.08 | (0.97-1.30) | 1.13 | 0.07 | (0.98-1.28) | 0.92 |

Ratio ½

| | | | | | | | | | | | |
|---|---|---|---|---|---|---|---|---|---|---|---|
| 2005 | Native pH4 −80 | NS502 (N = 33) | 18 | 15 | 0.87 | 0.06 | (0.75-0.99) | 0.84 | 0.10 | (0.62-1.06) | 0.39 |
| 2005 | Native pH5 −80 | NS503 (N = 33) | 18 | 15 | 1.17 | 0.10 | (0.96-1.39) | 1.11 | 0.22 | (0.64-1.58) | 0.25 |
| 2003 CDsf | Fraction C pH4 | NS403 (N = 30) | 15 | 15 | 1.14 | 0.03 | (1.07-1.20) | 1.08 | 0.09 | (0.89-1.28) | 0.2372 |

Ratio ⅔

| | | | | | | | | | | | |
|---|---|---|---|---|---|---|---|---|---|---|---|
| 2005 | Native pH4 −80 | NS502 (N = 33) | 18 | 15 | 3.37 | 1.04 | (1.17-5.57) | 5.8 | 1.59 | (2.39-9.21) | 0.01 |
| 2005 | Native pH5 −80 | NS503 (N = 33) | 18 | 15 | 1.73 | 0.31 | (1.07-2.39) | 2.27 | 0.40 | (1.42-3.12) | 0.21 |
| 2003 CDsf | Fraction C pH4 | NS403 (N = 30) | 15 | 15 | 0.99 | 0.05 | (0.89-1.10) | 1.13 | 0.10 | (0.91-1.35) | 0.42 |

TABLE 27
Estimation on continuous data (as signal-to-noise)
pAUC of 8% signifies Partial Area Under Curve, a rate of false positives (FP) of 8% was
imposed on the Dataset. The AUC, the variance of the AUCs and the pAUC were
calculated with a confidence interval (CI) of 95%

| AUC Empirical estimation | | | | | | | | |
|---|---|---|---|---|---|---|---|---|
| Date | Condition | Dataset | CG | DS | Auc (05) | Std. Err. | [95% CI] | pAuc (8%) |
| | | | | | Ratio ½ (Variable: Signal-to-noise) (SN) | | | |
| 2005 | Native pH5 −80 | NS503 (N = 33) | 18 | 15 | 0.38 | 0.11 | (0.17-0.60) | 0.30 |
| 2005 | Native pH4 −80 | NS502 (N = 33) | 18 | 15 | 0.41 | 0.11 | (0.20-0.62) | 0.33 |
| 2003 CDsf | Fraction C pH4 | NS403 (N = 30) | 15 | 15 | 0.37 | 0.11 | (0.15-0.59) | 0.29 |
| | | | | | Ratio ⅓ (Variable: Signal-to-noise) (SN) | | | |
| 2005 | Native pH5 −80 | NS503 (N = 33) | 18 | 15 | 0.60 | 0.11 | (0.39-0.80) | 0.52 |
| 2005 | Native pH4 −80 | NS502 (N = 33) | 18 | 15 | 0.76 | 0.09 | (0.59-0.93) | 0.68 |
| 2003 CDsf | Fraction C pH4 | NS403 (N = 30) | 15 | 15 | 0.51 | 0.11 | (0.29-0.73) | 0.43 |
| | | | | | Ratio ⅔ (Variable: Signal-to-noise) (SN) | | | |
| 2005 | Native pH5 −80 | NS503 (N = 33) | 18 | 15 | 0.63 | 0.10 | (0.43-0.83) | 0.55 |
| 2005 | Native pH4 −80 | NS502 (N = 33) | 18 | 15 | 0.77 | 0.09 | (0.59-0.95) | 0.69 |
| 2003 CDsf | Fraction C pH4 | NS403 (N = 30) | 15 | 15 | 0.59 | 0.11 | (0.37-0.81) | 0.51 |

TABLE 28
Median test

| | | |
|---|---|---|
| NS403 CDsf | NS502 | NS503 |

Groups together

| | | | |
|---|---|---|---|
| ratio13 | 0.03 | 0.11 | 0.06 |
| ratio12 | 1.00 | 0.71 | 0.26 |
| ratio23 | 1.00 | 0.11 | 0.71 |

Control

| | | | |
|---|---|---|---|
| ratio13 | 0.13 | 0.29 | 0.34 |
| ratio12 | 0.62 | 1.00 | 0.62 |
| ratio23 | 0.62 | 1.00 | 0.62 |

Down syndrome

| | | | |
|---|---|---|---|
| ratio13 | 0.62 | 0.12 | 0.61 |
| ratio12 | 1.00 | 0.28 | 0.61 |
| ratio23 | 1.00 | 0.01 | 0.61 |

TABLE 29
UNIVARIED SCREENING TEST ON BINORMAL DATA WITH HNP or RATIOS
(Datasets NS503/NS502/NS403CDsf) with variable SN

| Principal judgment | | | | | | | |
|---|---|---|---|---|---|---|---|
| criterion | | | | | | | |
| signal-to-noise (SN) | | | | | | | |
| or intensity | | Diagnostic result (SN) | | | | | |
| On total numbers | | | | | | | |
| Dataset NS502 (n = 30) | | HNP | | | Ratios | | |
| Dataset cluster No. | | Cluster 3 | Cluster 4 | Cluster 5 | (Longitudinal ratio HNPx/HNPy) | | |
| 95% CI Exact | | HNP2 | HNP1 | HNP3 | Ratio12 | Ratio13 | Ratio23 |
| Sensitivity | Se | 0.73 | 0.79 | 0.84 | 0.88 | 0.92 | 0.96 |
| Specificity | Sp | 0.73 | 0.90 | 0.62 | 0.70 | 0.58 | 0.51 |
| Positive Predictive Value | PPV | 0.77 | 0.75 | 0.87 | 0.85 | 0.92 | 0.96 |
| Negative Predictive Value | NPV | 0.75 | 0.77 | 0.85 | 0.86 | 0.92 | 0.95 |
| 95% Simel CI's | | | | | | | |
| Likehood ratio (+) | LR+ | 2.15 | 1.95 | 4.11 | 3.14 | 6.2 | 10.5 |
| Likehood ratio (−) | LR− | 1.95 | 2.15 | 1.23 | 1.36 | 0.92 | 0.73 |

Note:
For this case-control study on total numbers, we eliminated from the dataset analyzed the samples
M8 M9 M16. The numbers are therefore 15 CTRL and 15 Down S.

| On common numbers with NS526 | | | | | | | |
|---|---|---|---|---|---|---|---|
| Dataset NS502 (n = 22) | | HNP | | | Ratios | | |
| Dataset cluster No. | | Cluster 3 | Cluster 4 | Cluster 5 | (Longitudinal ratio HNPx/HNPy) | | |
| 95% CI Exact | | HNP2 | HNP1 | HNP3 | Ratio 12 | Ratio 13 | Ratio 23 |
| Sensitivity | Se | 0.73 | 0.84 | 0.88 | 0.84 | 0.92 | 0.96 |
| Specificity | Sp | 0.69 | 0.74 | 0.64 | 0.67 | 0.56 | 0.50 |
| Positive Predictive Value | PPV | 0.73 | 0.74 | 0.82 | 0.8 | 0.89 | 0.93 |
| Negative Predictive Value | NPV | 0.78 | 0.84 | 0.89 | 0.85 | 0.93 | 0.96 |
| 95% Simel CI's | | | | | | | |
| Likehood ratio (+) | LR+ | 2.22 | 2.23 | 3.39 | 2.94 | 5.53 | 8.31 |
| Likehood ratio (−) | LR− | 1.8 | 1.73 | 1.22 | 1.42 | 0.92 | 0.74 |

The numbers are 11 CTRL and 11 Down S.

FIGS. 35 to 38 present the results of the statistical study NS502, carried out on native samples, conserved at −80° C., with anionic separation on a Q10 surface, under conditions of pH=4 (optimal conditions for obtaining indicative longitudinal ratios).

Under the conditions of the NS502 study, the ratios of the intensities of the peaks HNP-1/HNP-3, and HNP-2/HNP-3 are particularly discriminating markers for detecting pregnant women who may be carrying a fetus suffering from Down syndrome. This has the considerable advantage of being able to obtain excellent reproducibility of the results, by doing away with any possible problems of calibration and doing away with any possible problems of signal variability, defined both in terms of mass and in terms of intensity or signal-to-noise or any other variable (for example: Tof area, tof width, with tof being representative of the mass of the substance). The inventors noted, moreover, that the expression levels of the defensins HNP2, HNP1 and HNP3, and also the ratios between these various levels, correlate neither with the age of the pregnant woman, nor with the week of pregnancy, nor with any of the markers currently used (AFP, uE3 and HCG, in particular), nor with any of the other markers identified in the studies reported here (Q10 or H50 purification).

### G—Supplementary Studies Using Anionic Surfaces for Ionization of the Proteins

The statistical study NS502 also made it possible to identify other peaks relevant in terms of the diagnosis of Down syndrome. These peaks are listed in table 30 hereinafter.

TABLE 30
Other peaks visible by purifying the samples by anion exchange, that
can potentially be used for the prenatal diagnosis of DS

| Statistical | Experimental | | | Standard |
|---|---|---|---|---|
| study | conditions | Peak No. | Mean m/z | deviation |
| NS502 | −80° C., native | 15 | 4461.60 | 1.60 |
| (N = 33, | serum. | 17 | 4570.90 | 1.60 |
| including | Q10, pH4, | 18 | 4630.70 | 1.40 |
| 18 controls | CHCA, | 19 | 4714.30 | 1.60 |
| and 15 | ammonium | 21 | 4859.10 | 1.80 |
| Down S.) | acetate, laser | 44 | 8195.65 | 3.70 |
| | 160 baseline | 50 | 8905.85 | 2.70 |
| | auto | 51 | 9121.00 | 3.00 |
| | | 54 | 9700.50 | 3.80 |
| | | 60 | 12435.3 | 5 |

Among these peaks (or clusters), the peaks at 4859.10 Da and at 8195.65 Da correspond, respectively, to the peaks at 4860 Da and 8206 Da identified in the preliminary studies (see table 14 above).

The peaks mentioned in table 30 were also validated by visual analysis of the spectra. The spectra showing the peaks at 8195.7 Da and at 8905.9 Da, overexpressed in the case of trisomie 21, are presented in FIGS. 39 and 40, respectively. The ROC curves associated with all the peaks mentioned in table 30 are the subject of FIG. 41.

### H—Supplementary Studies Using Hydrophobic Surfaces for Ionization of the Proteins

### H-1. Supplementary Studies on H50 Surface

Several supplementary studies were carried out by purifying the samples on an H50 hydrophobic surface. The results of two of these studies are presented here. The samples on which these studies were carried out are listed in table 31 hereinafter, and the relevant peaks identified are summarized in table 32.

TABLE 31
DATASET NS526 - POPULATION TESTED - Native H50 −80° C.

| Sample No. | Sampling date | Status | Age | weight | wk + d | hcg_iu | MoMhcg | ue3_pg | MoMue3 |
|---|---|---|---|---|---|---|---|---|---|
| M10 | 26/11/2004 | CTRL | 35.00 | 46 | 16 + 1 | 43.90 | 1.57 | 720.0 | 0.74 |
| M11 | 27/11/2004 | CTRL | 32.00 | 62 | 16 + 4 | 58.80 | 2.65 | 1010.0 | 1.00 |
| M12 | 27/11/2004 | CTRL | 38.00 | 52 | 15 + 4 | 19.00 | 0.61 | 470.0 | 0.59 |
| M13 | 27/11/2004 | CTRL | 30.00 | 54 | 14 | 16.20 | 0.33 | 410.0 | 1.12 |
| M14 | 28/11/2004 | CTRL | 28.00 | 79 | 15 + 6 | 59.70 | 2.65 | 780.0 | 1.03 |
| M15 | 28/11/2004 | CTRL | 36.00 | 57 | 16 + 2 | 10.40 | 0.42 | 690.0 | 0.72 |
| M16 | 27/11/2004 | CTRL | 29.00 | | 16 + 2 | 44.50 | 1.95 | 520.0 | 0.55 |
| M17 | 27/11/2004 | CTRL | 24.00 | 59 | 17 | 13.50 | 0.65 | 1340.0 | 1.18 |
| M18 | 27/11/2004 | CTRL | 30.00 | 52 | 16 + 6 | 20.30 | 0.89 | 1340.0 | 1.18 |
| M5 | 26/11/2004 | CTRL | 25.00 | | 17 + 4 | 11.30 | 0.64 | 1190.0 | 0.94 |
| M9 | 26/11/2004 | CTRL | 28.00 | 71 | 15 + 5 | 20.70 | 0.82 | 700.0 | 0.92 |
| F1 | 18 02 04 | DS | 32 | | 14 + 6 | 109.8 | 3.28 | 310 | 0.54 |
| F10 | 23 03 04 | DS | 39 | | 15 + 0 | 101.8 | 3.21 | 580 | 0.94 |
| F11 | 25 03 04 | DS | 40 | | 16 + 1 | 16.8 | 0.71 | 620 | 0.68 |
| F12 | 02 04 04 | DS | 23 | | 16 + 2 | 67.5 | 2.95 | 600 | 0.64 |
| F13 | 29 04 04 | DS | 32 | | 15 + 0 | 57.9 | 1.82 | 420 | 0.68 |
| F2 | 28 01 04 | DS | 27 | | 15 + 1 | 67.9 | 2.24 | 400 | 0.63 |
| F3 | 30 03 04 | DS | 28 | | 17 + 5 | 48.3 | 2.8 | 33 | 0.02 |
| F6 | 02 05 04 | DS | 34 | | 14 + 2 | 209.4 | 5.18 | 490 | 1.12 |
| F7 | 09 03 04 | DS | 39 | | 16 + 4 | 92.3 | 4.29 | 810 | 0.79 |
| F8 | 16 03 04 | DS | 34 | 65 | 17 + 3 | 84 | 4.59 | 1040 | 0.86 |
| M6 | 26/11/2004 | DS | 31.00 | 81 | 16 + 3 | 27.30 | 1.41 | 870.0 | 0.98 |

| | | | | | | Other |
|---|---|---|---|---|---|---|
| Sample No. | afp_iu | MoMafp | risk | Karyotype | Outcome | information |
| M10 | 36.10 | 1.00 | 241 | 46, XX | | girl H |
| M11 | 50.20 | 1.53 | 550 | | | girl H |
| M12 | 25.10 | 0.80 | 242 | 46, XX | | girl H |
| M13 | 22.90 | 1.00 | 10000 | | | girl H |
| M14 | 22.20 | 0.88 | 420 | | | girl H |
| M15 | 23.60 | 0.71 | 1100 | | | boy H |
| M16 | 29.20 | 0.91 | 86 | 46, XY | | boy H |
| M17 | 41.30 | 1.15 | 10000 | | | girl H |
| M18 | 29.10 | 0.77 | 6500 | | | boy H |
| M5 | 46.30 | 1.22 | 10000 | | | girl H |
| M9 | 28.10 | 1.05 | 8200 | | | boy H |
| F1 | 13.1 | 0.51 | 1/5 | 47, XY, +21 | MIP | |
| F10 | 18.3 | 0.70 | 1/21 | 47, XY, +21 | MIP | |
| F11 | 24.8 | 0.79 | 1/235 | 47, XX, +21 | MIP | |
| F12 | 33.4 | 1.04 | 1/71 | 47, XY, +21 | MIP | |
| F13 | 11.1 | 0.42 | 1/40 | 47, XX, +21 | MIP | |
| F2 | 18.5 | 0.73 | 1/68 | 47, XX, +21 | MIP | deformity |
| F3 | 51.77 | 1.34 | 1/41 | 46, XX, der(14; 21) | MIP | no anat. path. |
| F6 | 18.3 | 0.79 | 1/91 | 47, XX, +21 | MIP girl | |
| F7 | 31.8 | 0.95 | 1/5 | 47, XX, +21 | MIP | |
| F8 | 22.3 | 0.62 | 1/5 | 47, XY, +21 | MIP | |
| M6 | 13.20 | 0.49 | 480 | 47, XX, +21 | MIP | |

TABLE 32
Peaks identified by purification on hydrophobic surface and
mass spectrometry Ciphergen

| | | | | | | Standard |
|---|---|---|---|---|---|---|
| Experimental conditions | Dataset | CG | Down S. | Peak No. | Mean m/z | deviation |
| H50 Water Acetonitrile 5% SPA | NS526c (N = 22) | 11 | 11 | 8 | 4068.11 | 1.43 |
| Laser 170 Baseline Auto | | | | 9 | 4075.90 | 1.11 |
| | | | | 11 | 4084.69 | 0.80 |
| | | | | 29 | 4084.69 | 1.20 |
| | | | | 35 | 8104.60 | 0.80 |
| | | | | 36 | 8124.69 | 2.08 |
| | | | | 37 | 8140.33 | 2.27 |
| | | | | 38 | 8156.66 | 1.21 |
| H50 Acetonitrile 5% SPA | NS526d (N = 22) | 11 | 11 | 1 | 2625.47 | 0.62 |
| Laser 180 Baseline 10 | | | | 20 | 4069.20 | 1.48 |
| | | | | 21 | 4076.05 | 0.93 |
| | | | | 24 | 4083.95 | 0.18 |
| | | | | 25 | 4084.70 | 0.50 |
| | | | | 26 | 4093.13 | 1.63 |
| | | | | 30 | 4320.79 | 1.18 |
| | | | | 42 | 4642.66 | 1.50 |
| | | | | 43 | 4658.47 | 1.47 |
| | | | | 45 | 6419.80 | 1.90 |
| | | | | 58 | 8109.15 | 2.46 |
| | | | | 59 | 8124.47 | 2.68 |
| | | | | 60 | 8140.61 | 2.70 |
| | | | | 61 | 8154.11 | 1.72 |
| | | | | 62 | 8269.10 | 6.70 |
| | | | | 63 | 8338.80 | 6.70 |
| | | | | 66 | 8610.30 | 8.70 |

Among these peaks (or clusters), the peaks at approximately 4068.5 Da, 4320.79 Da, 4619.80 Da, 8107 Da, 8269.10 Da and 8610.30 Da correspond, respectively, to the peaks at 4067 Da, 4317 Da, 6423 Da, 8112 Da, 8263 Da and 8627 Da, identified in the preliminary studies (see table 14 above), and the doublet at 4642.66 Da and 4658.47 Da corresponds to the peak at 4650 Da initially identified.

The statistical data relating to these peaks and to their use as potential markers for the prenatal diagnosis of Down syndrome are presented in tables 33 to 36 hereinafter.

The diagnostic values relating to these markers are indicated in tables 37 and 38, in which the following abbreviations are used: PPV=Positive Predictive Value; NPV=Negative Predictive Value; LR+=Likehood ratio(+); LR−=Likehood ratio(−).

TABLE 33
Descriptive statistics (molecular mass)

| | | | | | Standard | |
|---|---|---|---|---|---|---|
| Dataset | CG | DS | Peak No. | Mean | deviation | [95% confidence interval] |
| NS526c (N = 22) | 11 | 11 | 8 | 4067.10 | 0.31 | (4066.46-4067.74) |
| | | | 9 | 4074.89 | 1.11 | (4074.40-4075.38) |
| | | | 11 | 4083.68 | 0.17 | (4083.33-4084.04) |
| | | | 29 | 6418.6 | 0.25 | (6418.07-6419.12) |
| | | | 36 | 8123.69 | 0.44 | (8122.76-8124.61) |
| | | | 37 | 8139.33 | 0.48 | (8138.32-8140.33) |
| | | | 38 | 8155.65 | 0.26 | (8155.11-8156.18) |
| NS526d (N = 22) | 11 | 11 | 1 | 2624.46 | 0.13 | (2624.18-2624.73) |
| | | | 20 | 4068.19 | 0.31 | (4067.54-4068.85) |
| | | | 21 | 4075.04 | 0.20 | (4074.63-4075.46) |
| | | | 24 | 4082.94 | 0.04 | (4082.86-4083.02) |
| | | | 25 | 4084.75 | 0.11 | (4084.51-4084.99) |
| | | | 26 | 4092.12 | 0.35 | (4091.40-4092.85) |
| | | | 30 | 4319.79 | 0.25 | (4319.26-4320.31) |
| | | | 42 | 4641.65 | 0.32 | (4640.98-4642.31) |
| | | | 43 | 4657.46 | 0.31 | (4656.81-4658.12) |
| | | | 58 | 8108.14 | 0.53 | (8107.05-8109.23) |
| | | | 59 | 8123.47 | 0.57 | (8122.28-8124.66) |
| | | | 60 | 8139.60 | 0.58 | (8138.40-8140.80) |
| | | | 61 | 8153.11 | 0.37 | (8152.34-8153.87) |
| | | | 62 | 8269.09 | 1.43 | (8266.12-8272.06) |
| | | | 63 | 8338.75 | 1.44 | (8335.77-8341.74) |
| | | | 66 | 8610.28 | 1.85 | (8606.43-8614.13) |

NB: The peaks 36, 37 and 38 of the NS526c study represent multicharged proteins corresponding to the proteins revealed by the peaks 8, 9 and 11 of this same study. Similarly, the peaks 59, 60 and 61 of the NS526d study correspond to the peaks 20, 21 and 24.

TABLE 34
Descriptive statistics and U-Mann Whitney test (nonparametric) on SN judgment criterion
and on the Datasets NS526c/NS526d; markers other than HNP

| | | | Cluster | | Standard | | | Standard | | P Mann- |
|---|---|---|---|---|---|---|---|---|---|---|
| Dataset | CG | DS | No. | Mean CG | deviation | [95% CI] | Mean Down S. | deviation | [95% CI] | Whitney |
| NS526c (N = 22) | 11 | 11 | 8 | 1.63 | 0.23 | (1.11-2.15) | 0.94 | 0.12 | (0.68-1.20) | 0.03 |
| | | | 9 | 2.77 | 0.57 | (1.50-4.03) | 1.02 | 0.14 | (0.71-1.33) | 0.01 |
| | | | 11 | 3.64 | 0.92 | (1.59-5.69) | 1.94 | 0.53 | (0.75-3.13) | 0.08 |
| | | | 29 | 8.08 | 0.39 | (7.22-8.94) | 14.37 | 2.01 | (9.90-18.85) | 0.01 |
| | | | 36 | 18.01 | 4.21 | (8.62-27.40) | 7.30 | 1.08 | (4.90-9.70) | 0.01 |
| | | | 37 | 23.27 | 6.35 | (9.12-37.42) | 11.30 | 3.35 | (3.83-18.77) | 0.02 |
| | | | 38 | 10.11 | 2.30 | (4.99-15.23) | 6.56 | 2.45 | (1.10-12.03) | 0.07 |
| NS526d (N = 22) | 11 | 11 | 1 | 4.68 | 0.40 | (3.80-5.56) | 4.47 | 0.82 | (2.64-6.30) | 0.18 |
| | | | 20 | 2.99 | 0.45 | (1.98-4.00) | 1.42 | 0.38 | (0.57-2.27) | 0.02 |
| | | | 21 | 3.27 | 0.41 | (2.35-4.19) | 1.60 | 0.30 | (0.94-2.26) | 0.00 |
| | | | 24 | 4.31 | 0.64 | (2.88-5.73) | 2.22 | 0.65 | (0.78-3.66) | 0.03 |
| | | | 25 | 4.89 | 0.96 | (2.75-7.03) | 3.95 | 1.38 | (0.88-7.02) | 0.16 |
| | | | 26 | 2.14 | 0.38 | (1.30-2.99) | 1.74 | 0.55 | (0.51-2.97) | 0.20 |
| | | | 30 | 4.41 | 0.45 | (3.42-5.40) | 7.37 | 1.18 | (4.74-9.99) | 0.05 |
| | | | 42 | 6.50 | 1.59 | (2.96-10.05) | 9.84 | 1.94 | (5.53-14.15) | 0.18 |
| | | | 43 | 4.43 | 1.01 | (2.18-6.67) | 7.64 | 1.57 | (4.13-11.15) | 0.14 |
| | | | 58 | 21.47 | 2.79 | (15.24-27.70) | 10.74 | 2.57 | (5.03-16.46) | 0.02 |
| | | | 59 | 27.83 | 3.67 | (19.64-36.01) | 15.27 | 2.66 | (9.34-21.19) | 0.02 |
| | | | 60 | 31.36 | 4.90 | (20.44-42.28) | 22.02 | 5.44 | (9.90-34.14) | 0.11 |
| | | | 61 | 16.22 | 2.90 | (9.76-22.68) | 12.13 | 3.84 | (3.58-20.69) | 0.16 |
| | | | 62 | 5.02 | 0.58 | (3.73-6.30) | 3.47 | 0.85 | (1.59-5.36) | 0.06 |
| | | | 63 | 5.69 | 0.80 | (3.90-7.48) | 3.54 | 0.95 | (1.42-5.65) | 0.02 |
| | | | 66 | 27.26 | 1.92 | (22.98-31.54) | 36.97 | 4.15 | (27.72-46.22) | 0.05 |

TABLE 35
Median test (nonparametric) on intensity judgment criterion
and on Datasets NS526c/NS526d
Markers other than HNP

| | | | | | | Median test |
|---|---|---|---|---|---|---|
| Dataset | CG | DS | Peak No. | Mean m/z | Standard deviation | p Pearson Chi2 |
| NS526c (N = 22) | 11 | 11 | 8 | 4068.11 | 1.43 | 0.01 |
| | | | 9 | 4075.90 | 1.11 | 0.03 |
| | | | 11 | 4084.69 | 0.80 | 0.03 |
| | | | 29 | 6418.60 | 1.20 | 0.01 |
| | | | 35 | 8104.60 | 0.80 | 0.003 |
| | | | 36 | 8124.69 | 2.08 | 0.01 |
| | | | 37 | 8140.33 | 2.27 | 0.01 |
| | | | 38 | 8156.66 | 1.21 | 0.01 |
| NS526d (N = 22) | 11 | 11 | 1 | 2625.47 | 0.62 | 0.01 |
| | | | 20 | 4069.20 | 1.48 | 0.00 |
| | | | 21 | 4076.05 | 0.93 | 0.00 |
| | | | 24 | 4083.95 | 0.18 | 0.01 |
| | | | 25 | 4084.70 | 0.50 | 0.01 |
| | | | 26 | 4093.13 | 1.63 | 0.09 |
| | | | 30 | 4320.79 | 1.18 | 0.03 |
| | | | 42 | 4642.66 | 1.50 | 0.09 |
| | | | 43 | 4658.47 | 1.47 | 0.09 |
| | | | 45 | 6419.80 | 1.90 | 0.09 |
| | | | 58 | 8109.15 | 2.46 | 0.00 |
| | | | 59 | 8124.47 | 2.68 | 0.03 |
| | | | 60 | 8140.61 | 2.70 | 0.03 |
| | | | 61 | 8154.11 | 1.72 | 0.03 |
| | | | 62 | 8269.10 | 6.70 | 0.01 |
| | | | 63 | 8338.80 | 6.70 | 0.01 |
| | | | 66 | 8610.30 | 8.70 | 0.01 |

TABLE 36
Calculation of the Roc AUC (CJ: m/z) (nonparametric) on selection Dataset
NS526c/NS526d Markers other than HNP

| Dataset | CG | DS | Peak No. | Mean m/z | Standard deviation | AUC (10) | Var (AUC) | pAUC (8%) |
|---|---|---|---|---|---|---|---|---|
| NS526c (N = 22) | 11 | 11 | 8 | 4068.11 | 1.43 | 0.23 | 0.02 | 0.15 |
| | | | 9 | 4075.90 | 1.11 | 0.24 | 0.02 | 0.17 |
| | | | 11 | 4084.69 | 0.80 | 0.24 | 0.02 | 0.17 |
| | | | 29 | 6418.60 | 1.20 | 0.81 | 0.03 | 0.74 |
| | | | 35 | 8104.60 | 0.80 | 0.20 | 0.03 | 0.14 |
| | | | 36 | 8124.69 | 2.08 | 0.20 | 0.03 | 0.14 |
| | | | 37 | 8140.33 | 2.27 | 0.18 | 0.03 | 0.14 |
| | | | 38 | 8156.66 | 1.21 | 0.28 | 0.03 | 0.20 |
| NS526d (N = 22) | 11 | 11 | 1 | 2625.47 | 0.62 | 0.25 | 0.00 | 0.18 |
| | | | 20 | 4069.20 | 1.48 | 0.20 | 0.03 | 0.12 |
| | | | 21 | 4076.05 | 0.93 | 0.67 | 0.02 | 0.59 |
| | | | 24 | 4083.95 | 0.18 | 0.10 | 0.03 | 0.07 |
| | | | 25 | 4084.70 | 0.50 | 0.28 | 0.03 | 0.22 |
| | | | 26 | 4093.13 | 1.63 | 0.62 | 0.03 | 0.54 |
| | | | 30 | 4320.79 | 1.18 | 0.88 | 0.04 | 0.80 |
| | | | 42 | 4642.66 | 1.50 | 0.71 | 0.03 | 0.63 |
| | | | 43 | 4658.47 | 1.47 | 0.76 | 0.03 | 0.68 |
| | | | 58 | 8109.15 | 2.46 | 0.18 | 0.02 | 0.13 |
| | | | 59 | 8124.47 | 2.68 | 0.21 | 0.02 | 0.16 |
| | | | 60 | 8140.61 | 2.70 | 0.28 | 0.01 | 0.22 |
| | | | 61 | 8154.11 | 1.72 | 0.24 | 0.03 | 0.18 |
| | | | 62 | 8269.10 | 6.70 | 0.17 | 0.03 | 0.13 |
| | | | 63 | 8338.80 | 6.70 | 0.14 | 0.02 | 0.13 |
| | | | 66 | 8610.30 | 8.70 | 0.80 | 0.01 | 0.72 |

Said table 36 shows peaks for which the AUC is greater than 0.6, which indicates a significant over-expression, but also peaks for which the AUC is less than 0.4. This corresponds to an underexpression, which is also discriminating. In the latter case, the ROC curves presented in FIGS. 49 and 50 are inverted (graph of sensitivity as a function of specificity, instead of sensitivity as a function of 1-specificity), according to the conventional practice in the art.

TABLE 37
Diagnostic results (variable or CJ: SN or intensity) (binormal)

| | | Dataset peak No. | | | | | | | |
|---|---|---|---|---|---|---|---|---|---|
| Dataset NS526c (n = 30) | | Peak 8 | Peak 9 | Peak 11 | Peak 29 | Peak 35 | Peak 36 | Peak 37 | Peak 38 |
| M/Z | | 4068.11 | 4075.90 | 4084.69 | 6418.60 | 8104.60 | 8124.69 | 8140.33 | 8156.66 |
| 95% CI Exact | | | | | | | | | |
| Sensitivity | Se | 0.80 | 0.80 | 0.80 | 0.80 | 0.70 | 0.70 | 0.80 | 0.80 |
| Specificity | Sp | 0.75 | 0.75 | 0.75 | 0.92 | 0.83 | 0.83 | 0.83 | 0.83 |
| Positive predictive value | PPV | 0.73 | 0.73 | 0.73 | 0.89 | 0.78 | 0.78 | 0.80 | 0.80 |
| Negative predictive value | NPV | 0.82 | 0.82 | 0.82 | 0.85 | 0.77 | 0.77 | 0.83 | 0.83 |
| Simel 95% CI | | | | | | | | | |
| Ratio of probability (+) | LR+ | 3.20 | 3.20 | 3.20 | 9.67 | 4.20 | 4.20 | 4.80 | 4.80 |
| Ratio of probability (−) | LR− | 0.27 | 0.27 | 0.27 | 0.22 | 0.36 | 0.36 | 0.24 | 0.24 |

TABLE 38
Diagnostic results (variable or CJ: SN or intensity) (binormal)

| | | Dataset peak No. | | | | | | | | |
|---|---|---|---|---|---|---|---|---|---|---|
| Dataset NS526d (n = 30) | | Peak 1 | Peak 20 | Peak 21 | Peak 24 | Peak 25 | Peak 26 | Peak 30 | Peak 42 | Peak 43 |
| M/Z | | 2625.47 | 4069.20 | 4076.05 | 4083.95 | 4084.70 | 4093.13 | 4320.79 | 4642.66 | 4658.47 |
| 95% CI Exact | | | | | | | | | | |
| Sensitivity | Se | 0.8 | 0.8 | 0.8 | 0.8 | 0.8 | 0.8 | 0.8 | 0.8 | 0.8 |
| Specificity | Sp | 0.83 | 0.75 | 0.92 | 0.83 | 0.75 | 0.75 | 0.83 | 0.75 | 0.75 |
| Positive predictive value | PPV | 0.8 | 0.73 | 0.89 | 0.8 | 0.73 | 0.73 | 0.8 | 0.73 | 0.73 |
| Negative predictive value | NPV | 0.83 | 0.82 | 0.85 | 0.83 | 0.82 | 0.82 | 0.83 | 0.82 | 0.82 |
| Simel 95% CI | | | | | | | | | | |
| Ratio of probability (+) | LR+ | 4.8 | 3.2 | 9.6 | 4.8 | 3.2 | 3.2 | 4.8 | 3.2 | 3.2 |
| Ratio of probability (−) | LR− | 0.24 | 0.27 | 0.22 | 0.24 | 0.27 | 0.27 | 0.24 | 0.27 | 0.27 |

| | | Dataset peak No. | | | | | | |
|---|---|---|---|---|---|---|---|---|
| Dataset NS526d (n = 30) | | Peak 58 | Peak 59 | Peak 60 | Peak 61 | Peak 62 | Peak 63 | Peak 66 |
| M/Z | | 8109.15 | 8124.47 | 8140.61 | 8154.11 | 8269.10 | 8338.80 | 8610.30 |
| 95% CI Exact | | | | | | | | |
| Sensitivity | Se | 0.9 | 0.7 | 0.8 | 0.8 | 0.8 | 0.8 | 0.8 |
| Specificity | Sp | 0.83 | 0.83 | 0.83 | 0.75 | 0.75 | 1 (100%) | 0.83 |
| Positive predictive value | PPV | 0.82 | 0.78 | 0.8 | 0.73 | 0.73 | 1 | 0.8 |
| Negative predictive value | NPV | 0.91 | 0.77 | 0.83 | 0.82 | 0.82 | 0.86 | 0.83 |
| Simel 95% CI | | | | | | | | |
| Ratio of probability (+) | LR+ | 5.4 | 4.2 | 4.8 | 3.2 | 3.2 | FP = 0 | 4.8 |
| Ratio of probability (−) | LR− | 0.12 | 0.36 | 0.24 | 0.27 | 0.27 | 0.2 | 0.24 |

Note:
For this case-control study on total numbers, the samples M8 M9 M16 were eliminated from the dataset analyzed. The numbers are therefore 15 CTRL and 15 DS.

FIGS. 43 to 48 show spectra on which some of the peaks mentioned above in the cases of purification on H50 appear. FIGS. 49 and 50 present the data relating to each peak (of the NS526c study and of the NS526d study, respectively), in the form of distribution curves and of ROC curves. FIGS. 51 and 52 show the results for these new markers, in the form of box plots. The points outside the boxes represent the aberrant points.

### H-2. Supplementary Studies on C8-C18 Surface, Using a High-Resolution Mass Spectrometer

The results obtained in the NS526c and NS526d studies set out above were also confirmed by means of supplementary studies carried out using a high-resolution mass spectrometer. These experiments made it possible to confirm that the markers identified in the studies presented in section H-1 above are relevant, independently of the technology used.

The conditions for obtaining the spectra presented in FIG. 53 (study NP0408-LEP) are the following:

purification on a C8 or C18 hydrophobic surface, using the MB-HIC kits referenced 223324 (C8) and 223325 (C18) in the Bruker catalog.

Matrix: Bruker HCCA matrix

Spectrometer: Bruker Ultraflex

Experimental protocol and acquisitions: see table 39 below.

TABLE 39
Acquisition parameters common to the various C8 and C18 spectra

HighVoltageSupplies:IonSource1 = 25000
HighVoltageSupplies:IonSource2 = 23400
HighVoltageSupplies:Lens = 6000
HighVoltageSupplies:Reflector = 26300
HighVoltageSupplies:Reflector2 = 14000
HighVoltageSupplies:Polarity = 0
HighVoltageSupplies:LinearDetector = 1693
HighVoltageSupplies:PieDelay = 0
Digitizer:Timebase = 1
Digitizer:BandWidthLimit = 3
Digitizer:ElectronicGainLevel = 2
MassRange:MassStart = 880
MassRange:MassEnd = 25118.9
MassRange:Mode = 1
MatrixSuppression:Mode = 2
MatrixSuppression:DeflectionConstant = 0.0554137
MatrixSuppression:CutOffMass = 900
MatrixSuppression:GateStrength = 1
CID:Switch = 0
Calibration = ‘V3.0CCalibrator 1 1 0 0 −2147483648 2147483647
V1.0CHPCData Order 0
vCoeff V1.0VectorDouble 0 c2 0 c0 0 minMass 0 maxMass 0
bUse 0 endCHPCData V3.0CTOFCalibrationConstants
26271 0.9999999999999999 420.24145676678
1315083.8221388 −0.019237097616266 2
V3.0CTOFCalibrationConstants 0 0 0 0 0 −1’

It appears, on the spectra presented in FIG. 53, that the peaks identified with the low-resolution spectrometer can also be identified with a high-resolution spectrometer. Of course, the use of a spectrometer with higher resolution sometimes results in resolving of peaks which corresponded to combined peaks in low resolution. Details of the correspondences established between the peaks observed in low resolution and those observed in high resolution are given in tables 40 and 41 below.

TABLE 40
Correspondences between the peaks observed in the NS526c and NP0408-LEP studies

| | | | | Bruker | | | | | |
|---|---|---|---|---|---|---|---|---|---|
| | Ciphergen | | | C8 | | C18 | | | |
| | Mass | Ctrl > T21 | T21 > Ctrl | Mass | Fractions | Mass | Fractions | Ctrl > T21 | T21 > Ctrl |
| NS526c | 4067 | X | | 4068 | N | — | — | X | |
| | | | | 4068 | D (P + A) | — | — | | X |
| | 4074 | X | | — | — | — | — | | |
| | 4083 | X | | — | — | — | — | | |
| | 6418.6 | | X | 6418 | N | | | | X |
| | | | | | | 6418 | N | | X |
| | | | | 6420 | D | | | | X |
| | | | | | | 6418 | D | ~ | ~ |
| | | | | 6418 | D (P + A) | | | | X |
| | | | | | | 6418 | D (P − A) | X | |
| | 8123.7 | X | | 8127.35 | D (P + A) | | | | X |
| | | | | | | 8124.86 | D | | x |
| | | | | 8125 | N | | | | X |
| | | | | | | 8125 | N | | X |
| | | | | 8127.66 | D | | | | X |
| | | | | | | 8125.59 | D (P + A) | | X |
| | 8139.3 | X | | | | 8140 | N | ~ | ~ |
| | | | | 8140 | D | | | | X |
| | | | | | | 8142.61 | D | X | |
| | 8155.6 | X | | — | — | — | — | | |

Notations common to tables 40 and 41:
N = native;
X = Selection condition;
~ = equal intensities;
— = peak absent from the spectra;
D = fraction D = proteins or protein complexes of native masses greater than 100 kDa;
D (P + A) = Fraction D, albumin-associated proteins;
D (P − A) = Fraction D, protein-depleted.

TABLE 41
Correspondences between the peaks observed in the NS526d and NP0408-LEP studies

| | | | | Bruker | | | | | |
|---|---|---|---|---|---|---|---|---|---|
| | Ciphergen | | | C8 | | C18 | | | |
| | Mass | Ctrl > T21 | T21 > Ctrl | Mass | Fractions | Mass | Fractions | Ctrl > T21 | T21 > Ctrl |
| NS526d | 2624.5 | X | | 2620 | N | | | X | |
| | | | | | | 2620 | N | X | |
| | | | | 2620 | D | | | X | |
| | | | | | | 2620 | D | X | |
| | | | | 2630 | D | | | X | |
| | | | | 2620 | D (P − A) | | | X | |
| | | | | | | 2620 | D (P − A) | X | |
| | 4068.2 | X | | 4068 | N | — | — | X | |
| | | | | 4068 | D (P + A) | — | — | | X |
| | 4075 | X | | — | — | — | — | | |
| | 4082.9 | X | | — | — | — | — | | |
| | 4092.1 | X | | 4092 | N | | | X | |
| | | | | | | 4092 | N | X | |
| | | | | 4092 | D | | | X | |
| | | | | | | 4092 | D | X | |
| | | | | 4092 | D (P − A) | | | | X |
| | | | | | | 4092 | D (P − A) | X | |
| | 4319.8 | | X | — | — | — | — | | |
| | 4641.6 | | X | 4641 | N | | | | X |
| | | | | | | 4641 | N | ~ | ~ |
| | | | | 4643 | D | | | ~ | ~ |
| | | | | | | 4641 | D | ~ | ~ |
| | | | | 4643 | D (P − A) | | | | X |
| | 4657.5 | | X | 4654 | D | — | — | | X |
| | 8108.1 | X | | 8108 | D (P + A) | — | — | | X |
| | 8123.5 | X | | 8125 | N | | | | X |
| | | | | | | 8125 | N | | x |
| | | | | 8127.66 | D | | | | X |
| | | | | | | 8124.86 | D | | X |
| | | | | 8127.35 | D (P + A) | | | | X |
| | | | | | | 8125.59 | D (P + A) | | X |
| | 8139.6 | X | | | | 8140 | N | ~ | ~ |
| | | | | 8140 | D | | | | X |
| | | | | | | 8142.61 | D | X | |
| | 8153.1 | X | | — | — | — | — | | |

### I—Examples of Marker Combinations

By way of illustration, table 42 presents the performance levels (sensitivity, specificity, etc.) of a combination of two ratios between the HNP defensins, and of a combination of these same ratios with hcg, on the NS502 dataset. Of course, other combinations, potentially even more specific and sensitive, can be produced between the various markers identified by the inventors.

TABLE 42
Example of combination of markers in order to establish a prenatal
diagnostic test for trisomie 21

| | | | Ratio13 + | Current |
|---|---|---|---|---|
| Dataset NS502 (n = 30) | | Ratio13 + | Ratio23 | triple |
| Combination | | Ratio23 | with hcg (iu) | test |
| 95% CI Exact | | | | |
| Sensitivity | Se | 92.30% | 90.40% | 70% |
| Specificity | Sp | 80.40% | 81.60% | 93% |
| Roc | AUC | 0.84 | 0.835 | |
| Positive Predictive | PPV | 79.80% | 80.10% | |
| Value | | | | |
| Negative Predictive | NPV | 92.50% | 91.20% | |
| Value | | | | |
| 95% Simel CI's | | | | |
| Likehood ratio (+) | LR+ | 3.76 | 4.1 | |
| Likehood ratio (−) | LR− | 0.645 | 0.524 | |

1. A method for the in vitro prenatal screening of chromosomal aberrations, comprising a step of detecting, in a biological sample originating from a pregnant woman, at least a first marker chosen from: (i) the human neutrophil defensins HNP-2, HNP-1 and HNP-3, and/or (ii) the molecular species detectable by mass spectrometry with separation of anion exchange type, at the following molecular weights: 4461.60 Da; 4570.90 Da; 4630.70 Da; 4714.30 Da; 4859.10 Da; 8195.65 Da; 8905.85 Da; 9121.00 Da; 9700.50 Da, and/or (iii) the molecular species detectable by mass spectrometry with separation of hydrophobic type, at the following molecular weights: 2625.47 Da; 4068.5 Da; 4075.95 Da; 4084.3 Da; 4084.7 Da; 4093.13 Da; 4320.79 Da; 4642.66 Da; 4658.47 Da; 6419.80 Da; 8107 Da; 8124.5 Da; 8140.5 Da; 8155 Da; 8269.10 Da; 8338.80 Da; 8610.30 Da, said method also comprising a step of detecting at least a second marker chosen from groups (i), (ii), and (iii), or from group (iv) consisting of chorionic gonadotrophin hormone (hCG); hCG β-subunit; unconjugated estriol (uE3); alpha-fetoprotein (AFP); inhibitin A; and pregnancy-associated plasma protein (PAPP-A).

2. The method as claimed in claim 1 which comprises the detection, in a biological sample originating from a pregnant woman, of at least three independent markers chosen from groups (i) to (iv).

3. The method as claimed in claim 1 which comprises a step consisting in comparing the concentration of each of the markers detected, with reference values for the concentration of these markers in pregnant women carrying normal fetuses, and in pregnant women carrying fetuses suffering from a known chromosomal and/or genetic aberration.

4. The method as claimed in claim 1, which comprises a step of analyzing at least one spectrum obtained from a biological sample originating from a pregnant woman, and wherein the signal of at least 2 peaks is chosen from (i) the peaks corresponding to the human neutrophil defensins HNP-2, HNP-1 and HNP-3, and/or (ii) the peaks that can be observed by mass spectrometry with separation of anionic type, at the following molecular weights: 4461.60 Da; 4570.90 Da; 4630.70 Da; 4714.30 Da; 4859.10 Da; 8195.65 Da; 8905.85 Da; 9121.00 Da; 9700.50 Da, and/or (iii) the peaks that can be observed by mass spectrometry with separation of hydrophobic type, at the following molecular weights: 2625.47 Da; 4068.5 Da; 4075.95 Da; 4084.3 Da; 4084.7 Da; 4093.13 Da; 4320.79 Da; 4642.66 Da; 4658.47 Da; 6419.80 Da; 8107 Da; 8124.5 Da; 8140.5 Da; 8155 Da; 8269.10 Da; 8338.80 Da; and 8610.30 Da, is measured.

5. The method as claimed in claim 1 wherein the ratio between the concentrations of HNP-3 and of HNP-1 and/or HNP-2 in the sample is measured.

6. The method as claimed in claim 1 wherein the first marker detected is the sum of the defensins HNP-2, HNP-1 and HNP-3.

7. The method as claimed in claim 6 wherein the defensins are detected by an immunological technique.

8. The method as claimed in claim 1, wherein the level of expression of the defensins in the sample is measured by semiquantitative or quantitative nucleic acid amplification.

9. The method as claimed in claim 1, wherein the chromosomal aberrations screened are selected from the group consisting of Down syndrome (trisomie 21), Edwards syndrome (trisomie 18), Patau syndrome (trisomie 13), Turner syndrome, Klinefelter syndrome, fragile X syndrome and penta X syndrome.

10. The method as claimed in claim 9 wherein a chromosomal aberration that is the subject of the screening is Down syndrome.

11. The use of one or more human neutrophil defensins chosen from HND-1, HND-2 and HND-3, as a marker or markers for the prenatal diagnosis of a chromosomal aberration.

12. A kit for the prenatal diagnosis of chromosomal aberrations, comprising means required for carrying out a method as claimed in claim 1.

13. The kit as claimed in claim 12, comprising antibodies and/or aptamers specific for one or more of said markers.

14. The kit as claimed in claim 12, comprising, for at least one of said markers, a set of nonoverlapping specific antibodies.

15. The kit as claimed in claim 14, in which the set of nonoverlapping specific antibodies comprises a capture antibody, immobilized on a support, and a detection antibody.

16. The kit as claimed in claim 12, comprising primers for the amplification of nucleic acids encoding the defensins HNP-1 and/or HNP-2 and/or HNP-3.

17. The kit as claimed in claim 16, also comprising at least one probe for detecting the nucleic acid amplification product(s).


SEQUENCE LISTING (3 sequences)

SEQ ID NO: 1 - protein, 30 aa, Homo Sapiens
  PEPTIDE (1)...(30): Human neutrophil defensin-1
ACYCRIPACIAGERRYGTCIYQGRLWAFCC

SEQ ID NO: 2 - protein, 29 aa, Homo sapiens
  PEPTIDE (1)...(29): Human neutrophil defensin-2
CYCRIPACIAGERRYGTCIYQGRLWAFCC

SEQ ID NO: 3 - protein, 30 aa, Homo sapiens
  PEPTIDE (1)...(30): Human neutrophil defensin-3
DCYCRIPACIAGERRYGTCIYQGRLWAFCC